(19) United States
(12) Patent Application Publication
(10) Pub. No.: US 2026/0209826 A1
(43) Pub. Date: Jul. 23, 2026

(54) METHODS FOR ANALYZING NUCLEIC ACID CARGOS OF LIPID NUCLEIC ACID ASSEMBLIES

(71) Applicant: Intellia Therapeutics, Inc., Cambridge, MA (US)

(72) Inventors: Shreelekha Jaligama, Waltham, MA (US); Nicholas Nisbett, Waltham, MA (US); Kutralanathan Renganathan, Hopkinton, MA (US); Christine Kitsos, North Andover, MA (US); Kyle Bake, Medford, MA (US)

(21) Appl. No.: 19/140,782

(22) PCT Filed: Dec. 22, 2023
(86) PCT No.: PCT/US2023/085810

§ 371 Date: Jun. 18, 2025

Related U.S. Application Data
(60) Provisional application No. 63/434,812, filed on Dec. 22, 2022.

(51) Int. Cl. C12Q 1/6806; B01D 15/34; B01D 15/36; G01N 30/86; G01N 30/88

(52) U.S. Cl. CPC C12Q 1/6806; B01D 15/34; B01D 15/366; G01N 30/8665; G01N 30/88; G01N 2030/8827

(57) ABSTRACT

The present disclosure provides, among other things, methods and compositions for separating, and/or determining the concentration of, nucleic acids in a lipid nucleic acid assembly composition. In certain embodiments, the present disclosure provides methods and compositions for separating, and/or determining the concentration of, nucleic acids that utilize size exclusion chromatography (SEC) and/or ion pair chromatography. In certain embodiments, the present disclosure provides methods and compositions for separating, and/or determining the concentration of, nucleic acids in which nucleic acids are extracted from a lipid nucleic acid assembly composition by a process that includes ethanol precipitation. In various embodiments, the nucleic acids are encapsulated in lipid nanoparticles (LNPs). In various embodiments, the nucleic acids include a messenger RNA (mRNA) and a guide RNA (gRNA).

### CROSS-REFERENCE TO RELATED APPLICATIONS

This application claims the benefit of U.S. Provisional Application No. 63/434,812, filed Dec. 22, 2022, the content of which is hereby incorporated by reference in its entirety.

### BACKGROUND

Lipid nucleic acid assembly compositions, e.g., lipid nanoparticles (LNPs) with nucleic acid cargos, are useful for a variety of therapeutics and molecular biology applications. In some lipid nucleic acid assembly compositions, particularly for use in applications such as gene editing, multiple nucleic acids are present in the composition. Separation of nucleic acids of distinct sizes, and determination of the concentrations thereof, is useful in the characterization of such compositions.

### SUMMARY

The present disclosure provides, among other things, methods and compositions for separating, and/or determining the absolute and/or relative concentration of, nucleic acids in a lipid nucleic acid assembly composition. In certain embodiments, the present disclosure provides methods and compositions for separating, and/or determining the absolute and/or relative concentration of, nucleic acids by using size exclusion chromatography (SEC). In certain embodiments, the present disclosure provides methods and compositions for separating, and/or determining the absolute and/or relative concentration of, nucleic acids by using ion pair chromatography. In certain embodiments, the present disclosure provides methods and compositions for separating, and/or determining the absolute and/or relative concentration of, nucleic acids in which nucleic acids are extracted from a lipid nucleic acid assembly composition by a process that includes ethanol precipitation. In various embodiments, the nucleic acids include a messenger RNA (mRNA) and a guide RNA (gRNA). In various embodiments, the nucleic acids include a first messenger RNA (mRNA) and a guide RNA (gRNA). In various embodiments, the nucleic acids include a first messenger RNA (mRNA), a second messenger RNA, and a guide RNA (gRNA).

The present disclosure includes, among other things, the recognition that, in preparation of a drug substance or drug product that includes a lipid nucleic acid assembly composition, it is useful and/or necessary to determine the absolute and/or relative concentration of each nucleic acid thereof. The present disclosure further includes, among other things, the recognition that methods and compositions disclosed herein can be particularly advantageous for determining the absolute and/or relative concentrations of nucleic acids in lipid nucleic acid assembly compositions. The present disclosure further includes among other things the recognition that methods and compositions disclosed herein can be particularly advantageous for determining the absolute and/or relative concentrations of RNA molecules such as each of one or more gRNA molecules and one or more mRNA molecules (e.g., of one mRNA or of two mRNAs) present in a lipid nucleic acid assembly composition. In some embodiments, a lipid nucleic acid assembly composition includes RNA molecules encapsulated by lipid nanoparticles.

Various prior methods for separating, and/or determining the concentration of, nucleic acids have included organic solvent solutions that can result in turbidity and low recovery, e.g., when applied to lipid nucleic acid assembly compositions. In various embodiments, a nucleic acid extract (e.g., an RNA extract) of the present disclosure is advantageously free or substantially free of organic solvents.

Various prior methods for separating, and/or determining the concentration of, nucleic acids have included detergents that are detrimental to chromatography performance and robustness. In various embodiments, a nucleic acid extract (e.g., an RNA extract) of the present disclosure is advantageously free or substantially free of detergents, e.g., detergents that are incompatible with chromatographic technique such as size exclusion chromatography (SEC). In various embodiments, a nucleic acid extract (e.g., an RNA extract) of the present disclosure is advantageously free or substantially free of detergents, e.g., detergents that are incompatible with chromatographic technique such as ion pair chromatography.

Certain prior methods for determining the concentration of nucleic acids in a lipid nucleic acid assembly composition have been useful for determining total nucleic acid concentration but not for determining the presence or concentration of individual nucleic acid species of the composition. For example, a Ribogreen fluoresecence assay (e.g., as described in Goswami et al. Conjugation of Mannans to Enhance the Potency of Liposome Nanoparticles for the Delivery of RNA Vaccines. Pharmaceutics 2021, 13(2): 240) can provide total RNA concentration of an assayed lipid nucleic acid assembly composition but is not useful for determining the presence or concentration of individual RNA species of the composition. Other advantages of methods and compositions provided herein for separating, and/or determining the absolute and/or relative concentration of, nucleic acids include high resolution separation of distinct nucleic acids, high accuracy, high precision, high recovery, and robustness, including with small nucleic acids (e.g., sgRNA) and low concentrations of nucleic acid (e.g., total nucleic acid concentration below 100 μg/mL).

Yet another advantage of methods and compositions provided herein for separating, and/or determining the absolute and/or relative concentration of, nucleic acids is ease of lipid nucleic acid assembly composition preparation and/or procedural efficiency. Methods and compositions provided herein provide, among other things, reduction of the number and/or complexity of steps required for nucleic acid extraction as compared to various prior methods, in combination with superior results. For example, methods and compositions provided herein can provide efficient preparation of high-purity extracts of nucleic acid from complex lipid nucleic acid assembly compositions (e.g., lipid nucleic acid assembly compositions in which nucleic acids are encapsulated by LNPs) for use in chromatography.

In at least one aspect, the present disclosure provides a method of separating nucleic acid present in a lipid nucleic acid assembly composition, the method including: a) preparing a nucleic acid extract from the lipid nucleic acid assembly composition, wherein the preparation of the nucleic acid extract includes ethanol precipitation of the nucleic acid from the lipid nucleic acid assembly composition; and b) subjecting the nucleic acid extract to SEC, wherein the SEC separates the nucleic acid. In at least one aspect, the present disclosure provides a method including: a) preparing a nucleic acid extract from the lipid nucleic acid assembly composition, wherein the preparation of the nucleic acid extract includes ethanol precipitation of the nucleic acid from the lipid nucleic acid assembly composition; and b) subjecting the nucleic acid extract to SEC. In certain embodiments, the method further includes determining the total concentration of the nucleic acid present in the lipid nucleic acid assembly composition.

In at least one aspect, the present disclosure provides a method of determining the total concentration of nucleic acid present in a lipid nucleic acid assembly composition, the method including: a) preparing a nucleic acid extract from the lipid nucleic acid assembly composition, wherein the preparation of the nucleic acid extract includes ethanol precipitation of the nucleic acid from the lipid nucleic acid assembly composition; b) subjecting the nucleic acid extract to SEC, wherein the SEC separates the nucleic acid; and c) determining the total concentration of the nucleic acid in the lipid nucleic acid assembly composition.

In at least one aspect, the present disclosure provides a method of determining the total concentration of nucleic acid present in a lipid nucleic acid assembly composition, the method including: a) preparing a nucleic acid extract from the lipid nucleic acid assembly composition, wherein the preparation of the nucleic acid extract includes ethanol precipitation of the nucleic acid from the lipid nucleic acid assembly composition; b) subjecting the nucleic acid extract to SEC; and c) determining the total concentration of the nucleic acid in the lipid nucleic acid assembly composition.

In certain embodiments of methods provided herein, the nucleic acid present in the lipid nucleic acid assembly composition includes at least a first nucleic acid and a second nucleic acid, and wherein the SEC separates the first nucleic acid from the second nucleic acid. In certain embodiments, the method further includes determining the concentration of the first nucleic acid and/or the second nucleic acid in the lipid nucleic acid assembly composition. In certain embodiments, the nucleic acid present in the lipid nucleic acid assembly composition includes at least a first nucleic acid, a second nucleic acid, and a third nucleic acid. In certain embodiments, the nucleic acid present in the lipid nucleic acid assembly composition includes at least a first nucleic acid, a second nucleic acid, and a third nucleic acid, and the SEC separates the first nucleic acid from the second nucleic acid and/or the third nucleic acid.

In certain embodiments of methods provided herein, the first nucleic acid is a ribonucleic acid (RNA) molecule. In certain embodiments, the first nucleic acid is a messenger RNA (mRNA) molecule. In certain embodiments, the mRNA molecule encodes a polypeptide, optionally wherein the polypeptide includes an RNA-guided DNA binding agent, optionally wherein the RNA-guided DNA binding agent includes a nuclease, optionally wherein the nuclease is a Cas protein, optionally wherein the RNA-guided DNA binding agent is a fusion polypeptide. In certain embodiments, the first nucleic acid encodes a base editor. In certain embodiments, the base editor is a cytosine base editor. In certain embodiments, the base editor is an adenine base editor. In certain embodiments, the first nucleic acid includes 1000-7000 nucleotides. In certain embodiments, the second nucleic acid is an RNA molecule. In certain embodiments, the second nucleic acid is a guide RNA (gRNA) molecule, optionally wherein the gRNA molecule is an sgRNA molecule. In certain embodiments, the second nucleic acid includes 75-200 nucleotides, optionally wherein the second nucleic acid includes 80-120 nucleotides. In certain embodiments, the nucleic acid present in the lipid nucleic acid assembly composition consists or consists essentially of the first nucleic acid and the second nucleic acid.

In certain embodiments of methods provided herein, the total concentration of nucleic acid in the lipid nucleic acid assembly composition is the sum of the concentration of the first nucleic acid and the concentration of the second nucleic acid. In certain embodiments of methods provided herein, the concentration of the first nucleic acid and/or the second nucleic acid is determined according to a calibration curve, wherein the calibration curve represents the concentration of nucleic acid in a dilution series of at least one reference nucleic acid. In certain embodiments, the concentration of the first nucleic acid and the second nucleic acid are each determined, wherein the concentration of the first nucleic acid is determined according to a first calibration curve and the concentration of the second nucleic acid is determined according to a second calibration curve. In certain embodiments, the first nucleic acid is an mRNA molecule and the first calibration curve is a dilution series of a reference mRNA molecule. In certain embodiments, the second nucleic acid is a gRNA molecule and the second calibration curve is a dilution series of a reference gRNA molecule. In certain embodiments, the first calibration curve represents a first dilution series including concentrations of a reference mRNA molecule that encompass a range including 12 μg/mL and 60 μg/mL, and/or a range including 12 μg/mL and 36 μg/mL. In certain embodiments, the second calibration curve represents a second dilution series including concentrations of a reference gRNA molecule that encompasses a range including 8 μg/mL and 40 μg/mL, and/or a range including 8 μg/mL and 24 μg/mL. In certain embodiments, the first dilution series and second dilution series include concentrations such that pairwise addition of a concentration from the first dilution series and a concentration from the second dilution series yields total RNA concentrations that encompass a range including 20 μg/mL and 100 μg/mL, or a range including 20 μg/mL and 60 μg/mL

In certain embodiments of methods provided herein, the method includes determining a concentration of the first nucleic acid and/or the second nucleic acid, and/or a total nucleic acid concentration, of the lipid nucleic acid assembly composition, that is equal to or less than 2.5 mg/mL, 2.0 mg/mL, 1.5 mg/mL, 1.0 mg/mL, 0.5 mg/mL, 0.4 mg/mL, or 0.3 mg/mL. In certain embodiments of methods provided herein, the method includes determining a concentration of the first nucleic acid and/or the second nucleic acid, and/or a total nucleic acid concentration, where the total nucleic acid concentration is between about 0.5 mg/mL and about 2 mg/mL, optionally wherein the total nucleic acid concentration is between about 0.75 mg/mL and about 1.8 mg/mL. In certain embodiments of methods provided herein, the SEC produces a chromatographic profile in which USP resolution between a nucleic acid peak representing the first nucleic acid and a nucleic acid peak representing the second nucleic acid is equal to or greater than 1.5. In certain embodiments of methods provided herein, the preparation of the nucleic acid extract includes diluting the concentration of the nucleic acid extract to a midpoint of a calibration curve.

In certain embodiments of methods provided herein, the lipid nucleic acid assembly composition comprises, consists, or consists essentially of lipid nanoparticles (LNPs) including the nucleic acid. In certain embodiments, the lipid component of the LNPs includes (i) an amine lipid, (ii) a helper lipid, and (iii) a stealth lipid, optionally wherein the LNPs include a neutral lipid, optionally wherein the stealth lipid is a PEG lipid. In certain embodiments, the first nucleic acid is a messenger RNA (mRNA) molecule encoding an RNA-guided DNA binding agent, the second nucleic acid is a gRNA molecule, the lipid nucleic acid assembly composition comprises, consists, or consists essentially of LNPs including molecules of the first nucleic acid and the second nucleic acid, and the lipid component of the LNPs includes (i) an amine lipid, (ii) a helper lipid, (iii) a stealth lipid, and (iv) a neutral lipid, optionally wherein the stealth lipid is a PEG lipid.

In certain embodiments of methods provided herein, the total concentration of nucleic acid in the lipid nucleic acid assembly composition is the total concentration of RNA in the lipid nucleic acid assembly composition. In certain embodiments of methods provided herein, the ethanol precipitation includes: a) mixing the lipid nucleic acid assembly composition with ethanol to produce an ethanol mixture; b) centrifuging the ethanol mixture, wherein the centrifugation produces a nucleic acid pellet and an ethanol supernatant; and c) isolating the nucleic acid pellet from the ethanol, wherein the nucleic acid pellet includes precipitated RNA. In certain embodiments, the isolating of the pellet includes decanting supernatant and/or drying the nucleic acid pellet. In certain embodiments, the preparation of the nucleic acid extract includes resuspending the nucleic acid pellet in water to produce an aqueous nucleic acid solution. In certain embodiments, preparation of the nucleic acid extract includes heating the aqueous nucleic acid solution to a temperature between about 65° C. and about 90° C. for at least about 2 minutes to about 15 minutes, optionally wherein the preparation of the nucleic acid extract includes, after the heating, cooling the aqueous nucleic solution, optionally wherein the cooling is to 4° C. In certain embodiments of methods provided herein, the nucleic acid extract is free or substantially free of lipid, free or substantially free of buffer, and/or free or substantially free of detergents.

In certain embodiments of methods provided herein, the SEC is a High Performance Liquid Chromatography (HPLC). In certain embodiments of methods provided herein, subjecting the nucleic acid extract to SEC includes contacting the nucleic acid extract with an SEC substrate, optionally wherein the SEC substrate is, or is present in, an SEC column. In certain embodiments, subjecting the nucleic acid extract to SEC includes contacting the nucleic acid extract with an SEC column having internal diameter of 3 mm to 6 mm, optionally having an internal diameter of about 4 mm to about 5 mm, optionally having an internal diameter of about 4.6 mm internal diameter. In certain embodiments, subjecting the nucleic acid extract to SEC includes contacting the nucleic acid extract with an SEC column having a length of about 100 mm to about 400 mm, optionally having a length of about 300 mm length. In certain embodiments of methods provided herein, subjecting the nucleic acid extract to SEC includes contacting the nucleic acid extract with an SEC substrate and/or SEC column having a pore size of about 5 nm to about 50 nm, optionally wherein the pore size is about 12.5 nm to about 25 nm. In certain embodiments of methods provided herein, subjecting the nucleic acid extract to SEC includes contacting the nucleic acid extract with an SEC substrate and/or SEC column having a particle size of about 3 μm to about 5 μm, optionally a particle size of about 4 μm. In certain embodiments of methods provided herein, subjecting the nucleic acid extract to SEC includes contacting the nucleic acid extract with an SEC substrate and/or SEC column including silica, optionally wherein the SEC substrate and/or SEC column includes silica particles.

In certain embodiments of methods provided herein, subjecting the nucleic acid extract to SEC includes contacting the nucleic acid extract with an SEC substrate and/or SEC column compatible with a mobile phase having a pH of about 7 to about 8, optionally wherein the SEC substrate and/or SEC column is compatible with a mobile phase having a pH of about 7.3 to about 7.7, optionally wherein the SEC substrate and/or SEC column is compatible with a mobile phase having a pH of about 7.5. In certain embodiments of methods provided herein, the nucleic acid extract to SEC includes contacting the nucleic acid extract with an SEC substrate and/or SEC column compatible with a mobile phase including Tris-HCl, optionally wherein the SEC substrate and/or SEC column is compatible with a mobile phase including about 5 mM to about 50 mM Tris-HCl, optionally wherein the SEC substrate and/or SEC column is compatible with a mobile phase including about 20 mM Tris-HCl. In certain embodiments of methods provided herein, subjecting the nucleic acid extract to SEC includes contacting the nucleic acid extract with an SEC substrate and/or SEC column compatible with a mobile phase including about 25 mM to about 200 mM NaCl, optionally wherein the SEC substrate and/or SEC column is compatible with a mobile phase including about 150 mM NaCl.

In certain embodiments of methods provided herein, subjecting the nucleic acid extract to SEC includes contacting the nucleic acid extract with an SEC mobile phase having a pH between about 7 and about 8, optionally wherein the mobile phase has a pH of about 7.3 to about 7.7, optionally wherein the mobile phase has a pH of about 7.5. In certain embodiments of methods provided herein, subjecting the nucleic acid extract to SEC includes contacting the nucleic acid extract with an SEC mobile phase including Tris-HCl, optionally wherein the SEC mobile phase includes about 5 mM to about 50 mM Tris-HCl, optionally wherein the SEC mobile phase includes about 20 mM Tris-HCl. In certain embodiments of methods provided herein, subjecting the nucleic acid extract to SEC includes contacting the nucleic acid extract with an SEC mobile phase including NaCl, optionally wherein the SEC mobile phase includes about 25 mM to about 200 mM NaCl, optionally wherein the SEC mobile phase includes about 150 mM NaCl. In certain embodiments of methods provided herein, the method includes measuring absorbance of SEC eluate at 260 nm. In certain embodiments of methods provided herein, the SEC is carried out at a temperature of 20-40° C., optionally wherein the SEC is carried out at a temperature of 30° C.

In at least one aspect, the present disclosure provides a kit for separating nucleic acid present in a lipid nucleic acid assembly composition, the kit including a SEC substrate and at least one reagent for ethanol precipitation of the nucleic acid from the lipid nucleic acid assembly composition, optionally wherein the kit is for separating two or more nucleic acids, optionally wherein the two or more nucleic acid molecules are or include a gRNA and an mRNA. In certain embodiments, the SEC substrate is, or is present in, an SEC column. In certain embodiments, the SEC column has an internal diameter of 3 mm to 6 mm, optionally an internal diameter of about 4 mm to about 5 mm, optionally an internal diameter of about 4.6 mm internal diameter. In certain embodiments, the SEC column has a length of about 100 mm to about 400 mm, optionally a length of about 300 mm length. In certain embodiments, the SEC substrate and/or SEC column has a pore size of about 5 nm to about 50 nm, optionally wherein the pore size is about 12.5 nm to about 25 nm. In certain embodiments, the SEC substrate and/or SEC column has a particle size of about 3 μm to about 5 μm, optionally a particle size of about 4 μm. In certain embodiments, the SEC substrate and/or SEC column includes silica, optionally wherein the SEC substrate includes silica particles. In certain embodiments, the SEC substrate and/or SEC column is compatible with a mobile phase having a pH of about 7 to about 8, optionally a mobile phase having a pH of about 7.3 to about 7.7, optionally a mobile phase having a pH of about 7.5. In certain embodiments, the SEC substrate and/or SEC column is compatible with a mobile phase including about 25 mM to about 200 mM NaCl, optionally a mobile phase including about 150 mM NaCl.

In certain embodiments of kits provided herein, the kit includes a dilution series of one or more reference nucleic acids, optionally wherein the kit includes a dilution series of a reference gRNA molecule and/or a reference mRNA molecule. In certain embodiments of kits provided herein, the kit includes instructions for preparation of a nucleic acid extract by a process including ethanol precipitation. In certain embodiments of kits provided herein, the kit includes instructions for separating the two or more nucleic acids by SEC, optionally wherein the two or more nucleic acids include or consist of RNA molecules, optionally wherein the RNA molecules include or consist of a gRNA molecule and an mRNA molecule. In certain embodiments of kits provided herein, the kit includes instructions for determining the concentration of the two more nucleic acids and/or total nucleic acid concentration, optionally wherein the two or more nucleic acids include or consist of RNA molecules, optionally wherein the RNA molecules include or consist of a gRNA molecule and an mRNA molecule.

In at least one aspect, the present disclosure provides a method of separating nucleic acid present in a lipid nucleic acid assembly composition, the method including: a) preparing a nucleic acid extract from the lipid nucleic acid assembly composition, where the preparation of the nucleic acid extract includes ethanol precipitation of the nucleic acid from the lipid nucleic acid assembly composition; and b) subjecting the nucleic acid extract to ion pair chromatography, where the ion pair chromatography separates the nucleic acid. In at least one aspect, the present disclosure provides a method including: a) preparing a nucleic acid extract from the lipid nucleic acid assembly composition, wherein the preparation of the nucleic acid extract includes ethanol precipitation of the nucleic acid from the lipid nucleic acid assembly composition; and b) subjecting the nucleic acid extract to ion pair chromatography. In certain embodiments, the method further includes determining the total concentration of the nucleic acid present in the lipid nucleic acid assembly composition.

In at least one aspect, the present disclosure provides a method of determining the total concentration of nucleic acid present in a lipid nucleic acid assembly composition, the method including: a) preparing a nucleic acid extract from the lipid nucleic acid assembly composition, where the preparation of the nucleic acid extract includes ethanol precipitation of the nucleic acid from the lipid nucleic acid assembly composition; b) subjecting the nucleic acid extract to ion pair chromatography, where the ion pair chromatography separates the nucleic acid; and c) determining the total concentration of the nucleic acid in the lipid nucleic acid assembly composition.

In at least one aspect, the present disclosure provides a method of determining the total concentration of nucleic acid present in a lipid nucleic acid assembly composition, the method including: a) preparing a nucleic acid extract from the lipid nucleic acid assembly composition, wherein the preparation of the nucleic acid extract includes ethanol precipitation of the nucleic acid from the lipid nucleic acid assembly composition; b) subjecting the nucleic acid extract to ion pair chromatography; and c) determining the total concentration of the nucleic acid in the lipid nucleic acid assembly composition.

In certain embodiments, the nucleic acid present in the lipid nucleic acid assembly composition includes at least a first nucleic acid and a second nucleic acid, and where the ion pair chromatography separates the first nucleic acid from the second nucleic acid. In certain embodiments, the method further includes determining the concentration of the first nucleic acid and/or the second nucleic acid in the lipid nucleic acid assembly composition. In certain embodiments, the nucleic acid present in the lipid nucleic acid assembly composition includes at least a first nucleic acid, a second nucleic acid, and a third nucleic acid. In certain embodiments, the nucleic acid present in the lipid nucleic acid assembly composition includes at least a first nucleic acid, a second nucleic acid, and a third nucleic acid, and the SEC separates the first nucleic acid from the second nucleic acid and/or the third nucleic acid. In certain embodiments, the first nucleic acid is a ribonucleic acid (RNA) molecule. In certain embodiments, the first nucleic acid is a messenger RNA (mRNA) molecule. In certain embodiments, the mRNA molecule encodes a polypeptide, optionally where the polypeptide includes an RNA-guided DNA binding agent, optionally where the RNA-guided DNA binding agent includes a nuclease, optionally where the nuclease is a Cas protein, optionally where the RNA-guided DNA binding agent is a fusion polypeptide. In certain embodiments, the first nucleic acid encodes a base editor. In certain embodiments, the base editor is a cytosine base editor. In certain embodiments, the first nucleic acid includes 1000-7000 nucleotides. In certain embodiments, the second nucleic acid is an RNA molecule. In certain embodiments, the second nucleic acid is a guide RNA (gRNA) molecule, optionally where the gRNA molecule is an sgRNA molecule. In certain embodiments, the second nucleic acid includes 75-200 nucleotides, optionally where the second nucleic acid includes 80-120 nucleotides. In certain embodiments, the third nucleic acid is a ribonucleic acid (RNA) molecule. In certain embodiments, the third nucleic acid is a messenger RNA (mRNA) molecule. In certain embodiments, the mRNA molecule encodes a polypeptide, optionally where the polypeptide is a DNA glycosylase inhibitor, optionally where the DNA glycosylase inhibitor is UGI. In certain embodiments, the third nucleic acid includes 750-3000 nucleotides. In certain embodiments, the nucleic acid present in the lipid nucleic acid assembly composition includes (i) the first nucleic acid and the second nucleic acid, or (ii) the first nucleic acid, the second nucleic acid, and the third nucleic acid.

In certain embodiments in which a lipid nucleic acid assembly composition includes only a first nucleic acid and a second nucleic acid of the present disclosure, the total concentration of nucleic acid in the lipid nucleic acid assembly composition is the sum of the concentration of the first nucleic acid and the concentration of the second nucleic acid. In certain embodiments, (i) the concentration of the first nucleic acid and/or the second nucleic acid is determined according to a calibration curve, where the calibration curve represents the concentration of nucleic acid in a dilution series of at least one reference nucleic acid. In certain embodiments, the concentration of the first nucleic acid and the second nucleic acid are each determined, where the concentration of the first nucleic acid is determined according to a first calibration curve and the concentration of the second nucleic acid is determined according to a second calibration curve.

In certain embodiments in which a lipid nucleic acid assembly composition includes only a first nucleic acid, a second nucleic acid, and a third nucleic acid of the present disclosure, the total concentration of nucleic acid in the lipid nucleic acid assembly composition is the sum of the concentration of the first nucleic acid, the concentration of the second nucleic acid, and the concentration of the third nucleic acid. In certain embodiments, the concentration of the first nucleic acid, the second nucleic acid, and/or the third nucleic acid is determined according to a calibration curve, where the calibration curve represents the concentration of nucleic acid in a dilution series of at least one reference nucleic acid. In certain embodiments, the concentration of the first nucleic acid, the second nucleic acid, and the third nucleic acid are each determined, where the concentration of the first nucleic acid is determined according to a first calibration curve, the concentration of the second nucleic acid is determined according to a second calibration curve, and the concentration of the third nucleic acid is determined according to a third calibration curve.

In certain embodiments, the first nucleic acid is an mRNA molecule and the first calibration curve is a dilution series of a first reference mRNA molecule, optionally where the length and/or sequence of the first reference mRNA molecule is the same as the first nucleic acid or differs from the first nucleic acid by no more than 10%, no more than 5%, or no more than 1%. In certain embodiments, the second nucleic acid is a gRNA molecule and the second calibration curve is a dilution series of a reference gRNA molecule, optionally where the length and/or sequence of the reference gRNA molecule is the same as the second nucleic acid or differs from the second nucleic acid by no more than 10%, no more than 5%, or no more than 1%. In certain embodiments, the third nucleic acid, when present, is an mRNA molecule and the third calibration curve is a dilution series of a second reference mRNA molecule, optionally where the length and/or sequence of the second reference mRNA molecule is the same as the third nucleic acid or differs from the third nucleic acid by no more than 10%, no more than 5%, or no more than 1%.

In certain embodiments, the first calibration curve represents a first dilution series including concentrations of a first reference mRNA molecule that encompass a range including 10 μg/mL and 100 μg/mL. In certain embodiments, the second calibration curve represents a second dilution series including concentrations of a reference gRNA molecule that encompasses a range including 5 μg/mL and 100 μg/mL, and/or a range including 5 μg/mL and 50 μg/mL. In certain embodiments, the third calibration curve represents a third dilution series including concentrations of a second reference mRNA molecule that encompass a range including 10 μg/mL and 100 μg/mL. In certain embodiments, the first dilution series and second dilution series include concentrations such that total concentration from the first dilution series, second dilution series, and third dilution series yields total RNA concentrations that encompass a range including 20 μg/mL and 200 μg/mL, or a range including 20 μg/mL and 100 μg/mL.

In certain embodiments, the method includes determining a concentration of the first nucleic acid, the second nucleic acid, and/or the third nucleic acid, and/or a total nucleic acid concentration, of the lipid nucleic acid assembly composition, that is equal to or less than 2.5 mg/mL, 2.0 mg/mL, 1.5 mg/mL, 1.0 mg/mL, 0.5 mg/mL, 0.4 mg/mL, or 0.3 mg/mL. In certain embodiments, the method includes determining a concentration of the first nucleic acid, the second nucleic acid, and/or the third nucleic acid, and/or a total nucleic acid concentration, where the total nucleic acid concentration is between about 0.5 mg/mL and about 2 mg/mL, optionally where the total nucleic acid concentration is between about 0.75 mg/mL and about 1.8 mg/mL. In certain embodiments, the ion pair chromatography produces a chromatographic profile in which USP resolution between a nucleic acid peak representing the first nucleic acid and a nucleic acid peak representing the second nucleic acid is equal to or greater than 1, 1.5, 2, 2.5, or 3. In certain embodiments, the ion pair chromatography produces a chromatographic profile in which USP resolution between a nucleic acid peak representing the first nucleic acid and a nucleic acid peak representing the third nucleic acid is equal to or greater than 1, 1.5, 2.0, 2.5, 3.0, 3.5, 4.0, 4.5, or 5. In certain embodiments, the ion pair chromatography produces a chromatographic profile in which USP resolution between a nucleic acid peak representing the second nucleic acid and a nucleic acid peak representing the third nucleic acid is equal to or greater than 1, 1.5, or 2. In certain embodiments, the preparation of the nucleic acid extract includes diluting the concentration of the nucleic acid extract to a midpoint of a calibration curve.

In certain embodiments, the first nucleic acid is a messenger RNA (mRNA) molecule encoding a cytosine base editor, the second nucleic acid is a gRNA molecule, optionally where the gRNA molecule is an sgRNA molecule, and the third nucleic acid is an mRNA molecule encoding a DNA glycosylase inhibitor, optionally where the DNA glycosylase inhibitor is UGI. In certain embodiments, the first nucleic acid does not encode a DNA glycosylase inhibitor and/or does not encode UGI. In certain embodiments, wherein the cytosine base editor includes a DNA glycosylase inhibitor, optionally wherein the DNA glycosylase inhibitor is UGI.

In certain embodiments, the lipid nucleic acid assembly composition includes lipid nanoparticles (LNPs) including the nucleic acid. In certain embodiments, the lipid component of the LNPs includes (i) an amine lipid, (ii) a helper lipid, and (iii) a stealth lipid, optionally where the LNPs include a neutral lipid, optionally where the stealth lipid is a PEG lipid. In certain embodiments, the first nucleic acid is a messenger RNA (mRNA) molecule encoding an RNA-guided DNA binding agent. In certain embodiments, the first nucleic acid is a messenger RNA (mRNA) molecule encoding a cytosine base editor, the second nucleic acid is a gRNA molecule, the third nucleic acid is an mRNA molecule encoding a DNA glycosylase inhibitor, optionally where the DNA glycosylase inhibitor is uracil glycosylase inhibitor (UGI), the lipid nucleic acid assembly composition includes LNPs including molecules of the first nucleic acid and the second nucleic acid, and the lipid component of the LNPs includes (i) an amine lipid, (ii) a helper lipid, (iii) a stealth lipid, and (iv) a neutral lipid, optionally where the stealth lipid is a PEG lipid. In certain embodiments, the total concentration of nucleic acid in the lipid nucleic acid assembly composition is the total concentration of RNA in the lipid nucleic acid assembly composition.

In certain embodiments, the ethanol precipitation includes: a) mixing the lipid nucleic acid assembly composition with ethanol to produce an ethanol mixture; b) centrifuging the ethanol mixture, where the centrifugation produces a nucleic acid pellet and an ethanol supernatant; and c) isolating the nucleic acid pellet from the ethanol, where the nucleic acid pellet includes precipitated RNA. In certain embodiments, the isolating of the pellet includes decanting supernatant, pipetting away supernatant, and/or drying the nucleic acid pellet. In certain embodiments, the preparation of the nucleic acid extract includes resuspending the nucleic acid pellet in water to produce an aqueous nucleic acid solution. In certain embodiments, preparation of the nucleic acid extract includes heating the aqueous nucleic acid solution to a temperature between about 65° C. and about 90° C. for at least about 2 minutes to about 15 minutes, optionally where the preparation of the nucleic acid extract includes, after the heating, cooling the aqueous nucleic solution, optionally where the cooling is to 4° C. In certain embodiments, the nucleic acid extract is free or substantially free of lipid, free or substantially free of buffer, and/or free or substantially free of detergents.

In certain embodiments, the ion pair chromatography is a reverse phase chromatography. In certain embodiments, the ion pair chromatography is a High Performance Liquid Chromatography (HPLC). In certain embodiments, the ion pair chromatography is a reverse phase High Performance Liquid Chromatography (HPLC).

In certain embodiments, subjecting the nucleic acid extract to ion pair chromatography includes contacting the nucleic acid extract with an ion pair chromatography substrate, optionally where the ion pair chromatography substrate is, or is present in, an ion pair chromatography column. In certain embodiments, subjecting the nucleic acid extract to ion pair chromatography includes contacting the nucleic acid extract with an ion pair chromatography column having internal diameter of 1 mm to 4 mm, optionally having an internal diameter of about 1.5 mm to about 2.5 mm, optionally having an internal diameter of about 2.1 mm internal diameter. In certain embodiments, subjecting the nucleic acid extract to ion pair chromatography includes contacting the nucleic acid extract with an ion pair chromatography column having a length of about 50 mm to about 400 mm, optionally having a length of about 100 mm length. In certain embodiments, subjecting the nucleic acid extract to ion pair chromatography includes contacting the nucleic acid extract with an ion pair chromatography substrate and/or ion pair chromatography column having a pore size of about 100 Å to about 3000 Å, optionally where the pore size is about 1000 Å to about 2000 Å. In certain embodiments, subjecting the nucleic acid extract to ion pair chromatography includes contacting the nucleic acid extract with an ion pair chromatography substrate and/or ion pair chromatography column having a particle size of about 3 μm to about 5 μm, optionally a particle size of about 4 μm. In certain embodiments, subjecting the nucleic acid extract to ion pair chromatography includes contacting the nucleic acid extract with an ion pair chromatography substrate and/or ion pair chromatography column including silica, optionally where the ion pair chromatography substrate and/or ion pair chromatography column includes silica particles.

In certain embodiments, subjecting the nucleic acid extract to ion pair chromatography includes contacting the nucleic acid extract with an ion pair chromatography substrate and/or ion pair chromatography column compatible with at least one mobile phase including dibutylammonium acetate and/or triethylammonium acetate. In certain embodiments, the at least one mobile phase includes 25 mM to 300 mM dibutylammonium acetate, optionally where the mobile phase includes 50 mM dibutylammonium acetate. In certain embodiments, the at least one mobile phase includes 25 mM to 300 mM triethylammonium acetate, optionally where the mobile phase includes 100 mM triethylammonium acetate. In certain embodiments, at least one mobile phase includes 25% to 100% acetonitrile in water, optionally where the mobile phase includes about 50%, about 55%, about 60%, about 65%, about 70%, about 75%, about 80%, about 85%, about 90%, about 95%, or about 100% acetonitrile in water. In certain embodiments, at least one mobile phase includes about 50% to about 100% acetonitrile in water, or about 75% to about 100% acetonitrile in water. In certain embodiments, the method includes measuring absorbance of ion pair chromatography eluate at about 240 nm to about 280 nm or at about 250 nm to about 270 nm, optionally wherein the method includes measuring absorbance of ion pair chromatography eluate at about 240 nm, about 245 nm, about 250 nm, about 255 nm, about 260 nm, about 265 nm, about 270 nm, about 275 nm, or about 280 nm. In certain embodiments, the ion pair chromatography is carried out at a temperature of 20-80° C., optionally where the ion pair chromatography is carried out at a temperature of 65° C.

In at least one aspect, the present disclosure provides a kit for separating nucleic acid present in a lipid nucleic acid assembly composition, the kit including a ion pair chromatography substrate and at least one reagent for ethanol precipitation of the nucleic acid from the lipid nucleic acid assembly composition, optionally where the kit is for separating two or more nucleic acids, optionally where the two or more nucleic acid molecules are or include a gRNA and an mRNA, and/or for separating three or more nucleic acids, optionally where the three or more nucleic acid molecules are or include a gRNA, a first mRNA, and a second mRNA. In certain embodiments, the ion pair chromatography substrate is, or is present in, an ion pair chromatography column. In certain embodiments, the ion pair chromatography column has an internal diameter of 1 mm to 4 mm, optionally an internal diameter of about 1.5 mm to about 2.5 mm, optionally an internal diameter of about 2.1 mm internal diameter. In certain embodiments, the ion pair chromatography column has a length of about 50 mm to about 400 mm, optionally a length of about 100 mm length. In certain embodiments, the ion pair chromatography substrate and/or ion pair chromatography column has a pore size of about 100 Å to about 3000 Å, optionally where the pore size is about 1000 Å to about 2000 Å. In certain embodiments, the ion pair chromatography substrate and/or ion pair chromatography column has a particle size of about 3 μm to about 5 μm, optionally a particle size of about 4 μm. In certain embodiments, the ion pair chromatography substrate and/or ion pair chromatography column includes silica, optionally where the ion pair chromatography substrate includes silica particles. In certain embodiments, the ion pair chromatography substrate and/or ion pair chromatography column is compatible with a mobile phase including dibutylammonium acetate and/or triethylammonium acetate. In certain embodiments, the kit includes a dilution series of one or more reference nucleic acids, optionally where the kit includes a dilution series of a reference gRNA molecule, a first reference mRNA molecule, and/or a second reference mRNA molecule. In certain embodiments, the kit includes instructions for preparation of a nucleic acid extract by a process including ethanol precipitation. In certain embodiments, the kit includes instructions for separating the two or more nucleic acids. or three or more nucleic acids, by ion pair chromatography, optionally where the nucleic acids include RNA molecules, optionally where the RNA molecules include a gRNA molecule, a first mRNA molecule, and/or a second mRNA molecule. In certain embodiments, the kit includes instructions for determining the concentration of the two more nucleic acids, three or more nucleic acids, and/or total nucleic acid concentration, optionally where the nucleic acids include RNA molecules, optionally where the RNA molecules include a gRNA molecule, a first mRNA molecule, and/or a second mRNA molecule.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element. The term “or” is used in an inclusive sense, i.e., equivalent to “and/or,” unless the context clearly indicates otherwise.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 15%, 14%, 13%, 12%, 11%, 10%, 9%, 8%, 7%, 6%, 5%, 4%, 3%, 2%, 1%, or less of the referenced value.

APOBEC3A: As used herein, the term “APOBEC3A” refers to a cytidine deaminase such as the protein expressed by the human A3A gene. The APOBEC3A may have catalytic DNA editing activity. An amino acid sequence of APOBEC3A has been described (UniPROT accession ID: p31941). In some embodiments, the APOBEC3A protein is a human APOBEC3A protein and/or a wild-type protein. Variants include proteins having a sequence that differs from wild-type APOBEC3A protein by one or several mutations (i.e., substitutions, deletions, insertions), such as one or several single point substitutions. For instance, a shortened APOBEC3A sequence could be used, e.g. by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to an APOBEC3A reference sequence. The variant is “functional” in that it shows a catalytic activity of DNA editing. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

Base editor: As used herein, the term “base editor” refers to an agent that includes a polypeptide that is capable of deaminating a base within a DNA molecule and an RNA-guided nickase (e.g., Cas9 nickase). The base editor can be capable of deaminating a cytidine (C) in DNA or of deaminating an adenine (A) in DNA. For example, a base editor may include an RNA-guided nickase (e.g., Cas9 nickase, e.g., Nme2Cas9(D16A) or Spy2Cas9(D10A)) fused to a cytidine deaminase (e.g., an APOBEC3A deaminase (A3A)) by an optional linker. In some embodiments the Cas9 is a nickase and comprises an inactivating mutation within the RuvC subdomain. In some embodiments the Cas9 is a nickase and comprises an inactivating mutation within the HNH subdomain. In some embodiments, the Cas9 is nuclease inactive and comprises inactivating mutations within the RuvC and HNH subdomains. In some cases, a base editor that includes a cytidine deaminase also includes a DNA glycosylase inhibitor (e.g., UGI; i.e., includes one or more DNA glycosylase inhibitor domains, e.g., one or more UGI domains). In some cases, a base editor that includes a cytidine deaminase can be used in combination with a DNA glycosylase inhibitor (e.g., UGI) delivered in trans. In some embodiments, a base editor does not include a DNA glycosylase inhibitor (e.g., by mRNA encoding one or more DNA glycosylase inhibitor domains, e.g., one or more UGI domains). In various embodiments, an mRNA molecule of the present disclosure encodes a base editor.

Between or From: As used herein, the term “between” refers to content that falls between indicated upper and lower, or first and second, boundaries (or “bounds”), inclusive of the boundaries. Similarly, the term “from”, when used in the context of a range of values, indicates that the range includes content that falls between indicated upper and lower, or first and second, boundaries, inclusive of the boundaries.

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Cytidine Deaminase: As used herein, the term “cytidine deaminase” refers to a polypeptide or complex of polypeptides that is capable of cytidine deaminase activity, that is catalyzing the hydrolytic deamination of cytidine or deoxy cytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)). In some embodiments, variants of any known cytidine deaminase or APOBEC protein are encompassed. Variants include proteins having a sequence that differs from wild-type protein by one or several mutations (i.e., substitutions, deletions, insertions), such as one or several single point substitutions. For instance, a shortened sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to a reference sequence. The variant is “functional” in that it shows a catalytic activity of DNA editing.

Drug Product: As used herein, the term “drug product” refers to a finished, pharmaceutically acceptable dosage form that includes a drug substance, generally but not necessarily in combination with one or more pharmaceutically acceptable carriers. A non-limiting example of a drug product is a lipid nucleic acid assembly in a finished pharmaceutically acceptable dosage form.

Drug Substance: As used herein, the term “drug substance” refers to an active agent (such as an active pharmaceutical ingredient (API) of, or for use in, a drug product) that is intended to provide a pharmacological activity (e.g., for treatment of a disease or condition). A non-limiting example of a drug substance is a lipid nucleic acid assembly suitable for use in a finished pharmaceutically acceptable dosage form.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Fusion polypeptide: As used herein, the terms “fusion polypeptide” or “chimeric polypeptide” generally refer to a polypeptide including at least two portions or segments. Typically, a polypeptide containing at least two such portions is considered to be a fusion polypeptide if the two portions (1) are not included in nature in the same peptide (e.g., where the second portion is heterologous to the first portion), and/or (2) have been linked to one another through action of the hand of man. A fusion polypeptide can include amino acids in addition to amino acids of the two portions of the fusion polypeptide, or in addition to amino acids of the at least two portions of the polypeptide. The portions of a fusion polypeptide can be referred to as “fusion partners,” and as being “fused” or “linked.”

Guide RNA: “Guide RNA”, “gRNA”, and simply “guide” are used herein interchangeably to refer to the guide that directs an RNA-guided DNA binding agent to a target DNA and can be either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally occurring sequence, or a trRNA sequence with modifications or variations compared to naturally-occurring sequences.

Guide Sequence: As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. A guide sequence can be 24 base pairs in length, e.g., in the case of Neisseria meningitidis (i.e., Nme Cas9) and related Cas9 homologs/orthologs (e.g., Nme2Cas9(D16A)). Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

Heterologous: The term “heterologous,” as used herein, means a nucleotide or polypeptide sequence that is not found in the native nucleic acid or protein, respectively. For example, in a chimeric Cas9/Csnl protein, the RNA-binding domain of a naturally-occurring bacterial Cas9/Csnl polypeptide (or a variant thereof) may be fused to a heterologous polypeptide sequence (i.e., a polypeptide sequence from a protein other than Cas9/Csnl or a polypeptide sequence from another organism). The heterologous polypeptide sequence may exhibit an activity (e.g., enzymatic activity) that will also be exhibited by the chimeric Cas9/Csnl protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e., a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

Improve, increase, inhibit, decrease or reduce: As used herein, the terms “improve”, “increase”, “inhibit”, “decrease” and “reduce”, and grammatical equivalents thereof, indicate qualitative or quantitative difference from a reference.

Isolated or purified: As used herein, the terms “isolated”, “purified”, or “separated” refer to a substance and/or entity that has been (a) separated from at least some of the components with which it was associated when initially produced (whether in nature and/or in an experimental setting), and/or (b) designed, produced, prepared, and/or manufactured by the hand of man. Isolated, purified, or separated substances and/or entities may be separated from at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated, purified, or separated substances and/or entities are at least about 80%, about 85%, about 90%, about 91%, about 92%, about 93%, about 94%, about 95%, about 96%, about 97%, about 98%, about 99%, or more than about 99% pure. As used herein, a substance and/or entity is “pure” if it is substantially free of other components. In some embodiments, as will be understood by those skilled in the art, a substance and/or entity may still be considered “isolated”, “separated”, or “pure” after having been combined with certain other components such as, for example, one or more carriers or excipients (e.g., buffer, solvent, water, etc.); in such embodiments, percent isolation, separation, or purity of the substance and/or entity is calculated without including such carriers or excipients. To give but one example, in some embodiments, a polynucleotide of a lipid nucleic acid assembly composition can be referred to as “isolated”, “separated”, or “purified” when it is free, substantially free, or at least about 80% free (e.g., about 85%, about 90%, about 91%, about 92%, about 93%, about 94%, about 95%, about 96%, about 97%, about 98%, about 99%, or more than about 99% free) of one or more, or all, of the lipid components of the lipid nucleic acid assembly composition. Alternatively or additionally, in some embodiments, a polypeptide or polynucleotide that has been subjected to one or more isolation, separation or purification techniques may be considered to be an “isolated”, “separated”, or “purified” polypeptide or polynucleotide to the extent that it has been separated from other components (a) with which it is associated in nature; and/or (b) with which it had been previously associated, e.g., when initially produced.

Messenger RNA: “Messenger RNA” or “mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Polynucleotide or Nucleic Acid: “Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 0⁶-methylguanine, 4-thio-pyrimidines, 4-amino-pyrimidines, 4-dimethylhydrazine-pyrimidines, and 0⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicycbc furanose unit locked in an RNA mimicking sugar conformation, which enhance hybridization affinity toward complementary RNA and DNA sequences (V ester and Wengel, 2004, Biochemistry 43(42): 13233-41). RNA and DNA have different sugar moieties and can differ by the presence of uracil or analogs thereof in RNA and thymine or analogs thereof in DNA. In some embodiments, a nucleic acid has a sequence that is or encodes a functional gene product. In some embodiments a nucleic acid is or encodes a gRNA. In some embodiments a nucleic acid is or encodes an mRNA. In some embodiments a nucleic acid encodes a protein.

For the avoidance of doubt, each reference to a nucleic acid (including, e.g., an sgRNA and/or an mRNA) as disclosed herein, except as otherwise contextually defined, encompasses (i) a single nucleic acid molecule, (ii) a population of nucleic acid molecules having the same particular nucleic acid sequence, (iii) a population of nucleic acid molecules each having the same particular nucleic acid sequence or a sequence that is at least, e.g., 95%, 96%, 97%, 98%, 99%, or 100% identical to the particular nucleic acid sequence, and/or (iv) a population of nucleic acid molecules each having the same particular nucleic acid sequence or a sequence that differs from the particular nucleic acid sequence at no more than, e.g., 1, 2, 3, 4, or 5 nucleotide positions (e.g., by a nucleotide addition, deletion, or substitution). It is to be generally appreciated that lipid nucleic acid assembly compositions and nucleic acid extracts of the present disclosure include populations of nucleic acid molecules, in amounts detectable according to techniques disclosed herein.

Nucleic Acid Extract: As used herein, the term “nucleic acid extract” refers to a composition that includes nucleic acid isolated from a lipid nucleic acid assembly composition. The present disclosure includes that a nucleic acid extract can be the direct product of a process of nucleic acid extraction (a “primary extract”) or can be a composition produced or obtained by processing of a primary extract, e.g., by addition or removal of one or more agents, e.g., by dilution or concentration of a primary extract and/or by combination of a primary extract with reagents to facilitate analysis.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical formulation: As used herein, the term “pharmaceutical composition” or “formulation” refers to a composition in which a therapeutic agent is formulated together with one or more pharmaceutically acceptable carriers.

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, cell, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative or qualitative. Typically, as would be understood by those of skill in the art, a reference and the value to which it is compared represent assessments under comparable conditions. Those of skill in the art will appreciate when sufficient similarities are present to justify reliance on and/or comparison. In some embodiments, an appropriate reference may be an agent, sample, sequence, cell, subject, animal, or individual, or population thereof, under conditions those of skill in the art will recognize as comparable, e.g., for the purpose of assessing one or more particular variables (e.g., presence, absence, or absolute and/or relative concentration of, an agent or condition), or a measure or characteristic representative thereof.

RNA-guided DNA binding agent: As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas 10, Csml, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpfl, C2cl, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Class 2 Cas nucleases include, e.g., the Nme2Cas9(D16A) nickase. Several Cas9 orthologs have been obtained from N. meningitidis (Esvelt et al., NAT. METHODS, vol. 10, 2013, 1116-1121; Hou et al., PNAS, vol. 110, 2013, pages 15644-15649; Edraki et al., Mol. Cell 73:714-726, 2019) (NmelCas9, Nme2Cas9, and Nme3Cas9). The Nme2Cas9 ortholog functions efficiently in mammalian cells, recognizes an N4CC PAM, and can be used for in vivo editing (Ran et al., NATURE, vol. 520, 2015, pages 186-191; Kim et al., NAT. COMMUN., vol. 8, 2017, pages 14500). Nme2Cas9 has been shown to be naturally resistant to off-target editing (Lee et al., MOL. THER., vol. 24, 2016, pages 645-654; Kim et al., 2017). See also e.g, WO/2020081568 (e.g, pages 28 and 42), describing an Nme2Cas9 D16A nickase, the contents of which are hereby incorporated by reference in its entirety. Cpfl protein, Zetsche et al., Cell, 163: 1-13 (2015), is homologous to Cas9, and contains a RuvC-like nuclease domain. Cpfl sequences of Zetsche are incorporated by reference in their entirety. See, e.g., Zetsche, Tables SI and S3. See, e.g., Makarova et al., Nat Rev Microbiol, 13(11): 722-36 (2015); Shmakov etak, Molecular Cell, 60:385-397 (2015).

Sample: As used herein, the term “sample” typically refers to an aliquot of material obtained or derived from a source of interest. In some embodiments, a source of interest is a biological or environmental source. In some embodiments, a sample is a “primary sample” obtained directly from a source of interest. In some embodiments, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation, separation, and/or purification of certain components, etc.

Numeric ranges are inclusive of the numbers defining the range. Measured and measurable values are understood to be approximate, taking into account significant digits and the error associated with the measurement.

### BRIEF DESCRIPTION OF THE DRAWINGS

FIG. 1 is a schematic of an exemplary process for nucleic acid extraction that includes ethanol precipitation, such as in Example 1.

FIG. 2 is an exemplary chromatogram showing that SEC according to Example 2 achieves robust separation of mRNA and sgRNA.

FIG. 3 is an exemplary chromatographic profile produced by SEC of a reference nucleic acid composition including mRNA and sgRNA (total nucleic acid concentration 40 μg/mL) according to WS3 as set forth in Example 2, showing robust separation of mRNA and sgRNA.

FIG. 4 is an exemplary chromatographic profile produced by SEC of a nucleic acid extract including an mRNA and an sgRNA, prepared from a lipid nucleic acid assembly composition including LNPs and diluted to have a total nucleic acid concentration of 40 μg/mL. The chromatographic profile shows robust separation of mRNA and sgRNA.

FIG. 5 is a schematic of an exemplary process for nucleic acid extraction that includes ethanol precipitation, such as in Example 3.

FIG. 6 is an exemplary chromatogram showing that the Ion-Pair Reverse Phase (IP RP) chromatography according to Example 4 achieves robust separation of three RNA species including a ~0.1 kb sgRNA, a ~1.0 kb mRNA, and a ~4.5 kb mRNA (the three RNA species having a total nucleic acid concentration 100 μg/mL).

FIG. 7 is an exemplary chromatogram showing that IP RP chromatography according to Example 4 achieves robust separation of three RNA species including a ~0.1 kb sgRNA, a ~1.0 kb mRNA, and a ~4.5 kb mRNA (the three RNA species having a total nucleic acid concentration 12.5 μg/mL).

FIG. 8 is an exemplary chromatographic profile showing an overlay of three different samples separated according to Example 4: (1) a standard including three RNA species (a ~0.1 kb sgRNA, a ~1.0 kb mRNA, and a ~4.5 kb mRNA), the three RNA species having a total nucleic acid concentration of 50 μg/mL, (2) a first replicate of separation of three mRNA species by IP RP chromatography (LNP extraction A), and (3) a second replicate of separation of three mRNA species by IP RP chromatography (LNP extraction B). The chromatographic profiles show robust separation of three RNA species present in the LNP extractions which include a ~0.1 kb sgRNA, a ~1.0 kb mRNA, and a ~4.5 kb mRNA.

### DETAILED DESCRIPTION

The present disclosure provides methods of separating, quantifying, and/or characterizing nucleic acids present in a lipid nucleic acid assembly composition. In various embodiments, the lipid nucleic acid assembly composition includes LNPs that encapsulate nucleic acids (e.g., RNA). In various embodiments, the lipid nucleic acid assembly composition includes sgRNA, mRNA, or both. In various embodiments, methods of separating, quantifying, and/or characterizing nucleic acids present in a lipid nucleic acid assembly composition include extraction of nucleic acid from the lipid nucleic acid assembly composition. In particular embodiments, nucleic acids are extracted from a lipid nucleic acid assembly composition by ethanol precipitation, isopropyl alcohol precipitation, butanol precipitation, or acetonitrile precipitation. In various embodiments, methods provided herein are useful, e.g., to separate, quantify, and/or characterize nucleic acids present in a lipid nucleic acid assembly composition in which the nucleic acids are encapsulated by LNPs because the precipitation (e.g., ethanol precipitation) produces a nucleic acid extract that is free or substantially free of other materials such as lipids and/or detergents. In some embodiments, the extracted nucleic acid can be analyzed by a chromatographic method, such as size exclusion chromatography (SEC), or ion pair chromatography. In various embodiments, multiple nucleic acids in a nucleic acid extract can be separated by SEC. In various embodiments, multiple nucleic acids in a nucleic acid extract can be separated by ion pair chromatography. In various embodiments quantification can include determining the concentration (e.g., an absolute and/or relative concentration) of nucleic acids present in a lipid nucleic acid assembly composition, ratio of nucleic acids present in a lipid nucleic acid assembly composition, and/or total concentration (e.g., an absolute and/or relative total concentration) of nucleic acid present in a lipid nucleic acid assembly composition.

### Exemplary Lipid Nucleic Acid Assembly Compositions

Provided herein are various embodiments that make use of lipid nucleic acid assemblies, e.g., lipid nanoparticles, comprising one or more (e.g., one, two, three, four, five, six, seven, eight, nine, ten or more) distinct nucleic acid molecules. The methods and kits provided herein allow the rapid assessment of said nucleic acid molecules without interference from the lipid components of the lipid nucleic acid assemblies, or from detergents that have traditionally been used for extraction of nucleic acids. In some embodiments, one or more of said nucleic acid molecules comprise or consist of RNA. In some embodiments, the lipid nucleic acid assemblies can comprise genome editing tools, such as RNAs, including CRISPR/Cas components and RNAs that encode and/or express the same. A lipid nucleic acid assembly composition may comprise a nucleic acid, e.g., an RNA, component that includes one or more of a mRNA encoding an RNA-guided DNA-binding agent, a Cas nuclease mRNA, a Class 2 Cas nuclease mRNA, a Cas9 mRNA, a mRNA encoding a base editor, an mRNA encoding a fusion protein comprising an RNA-guided DNA-binding agent, and/or a gRNA. In some embodiments, a lipid nucleic acid assembly composition may include two RNA molecules. For example, in some embodiments, the lipid nucleic acid assembly composition can include (i) a mRNA encoding a Class 2 Cas nuclease and (ii) a gRNA. In some embodiments, a lipid nucleic acid assembly composition may include (i) mRNA encoding a base editor and (ii) a gRNA. In some embodiments, a lipid nucleic acid assembly composition may include (i) mRNA encoding a cytosine base editor, (ii) mRNA encoding a DNA glycosylase inhibitor (e.g., UGI), and (iii) a gRNA. In some embodiments, lipid nucleic acid assemblies include an RNA that encodes and/or expresses a base editor that includes a deaminase associated with a DNA binding domain such as a catalytically impaired nuclease domain (e.g., catalytically impaired CRISPR/Cas). In some embodiments, lipid nucleic acid assemblies include an RNA that encodes and/or expresses a DNA glycosylase inhibitor (e.g., uracil DNA glycosylase inhibitor (UGI)).

As used herein, “lipid nucleic acid assembly composition” refers to lipid-based delivery compositions, including lipid nanoparticles (LNPs) and lipoplexes. In some embodiments, “LNP compositions” are used interchangeably with “LNPs” or “LNP.”

In some embodiments, LNP refers to lipid nanoparticles with a diameter of <100 nanometers (nm), or a population of LNP with an average diameter of <100 nanometers (nm). In other embodiments, an LNP can have a diameter of about 1-250 nm, about 1-200 nm, about 1-175 nm, about 1-150 nm, about 1-125 nm, about 1-120 nm, about 1-100 nm, about 10-250 nm, about 10-200 nm, about 10-175 nm, about 10-150 nm, about 10-125 nm, about 10-120 nm, about 10-100 nm, about 20-250 nm, about 20-200 nm, about 20-175 nm, about 20-150 nm, about 20-125 nm, about 20-120 nm, about 20-100 nm, about 50-250 nm, about 50-200 nm, about 50-175 nm, about 50-150 nm, about 50-125 nm, about 50-120 nm, about 50-100 nm, about 60-250 nm, about 60-200 nm, about 60-175 nm, about 60-150 nm, about 60-125 nm, about 60-120 nm, about 60-100 nm, about 75-250 nm, about 75-200 nm, about 75-175 nm, about 75-150 nm, about 75-125 nm, about 75-120 nm, or about 75-100 nm. In certain embodiments, a population of LNP has an average diameter of about about 1-250 nm, about 1-200 nm, about 1-175 nm, about 1-150 nm, about 1-125 nm, about 1-120 nm, about 1-100 nm, about 10-250 nm, about 10-200 nm, about 10-175 nm, about 10-150 nm, about 10-125 nm, about 10-120 nm, about 10-100 nm, about 20-250 nm, about 20-200 nm, about 20-175 nm, about 20-150 nm, about 20-125 nm, about 20-120 nm, about 20-100 nm, about 50-250 nm, about 50-200 nm, about 50-175 nm, about 50-150 nm, about 50-125 nm, about 50-120 nm, about 50-100 nm, about 60-250 nm, about 60-200 nm, about 60-175 nm, about 60-150 nm, about 60-125 nm, about 60-120 nm, about 60-100 nm, about 75-250 nm, about 75-200 nm, about 75-175 nm, about 75-150 nm, about 75-125 nm, about 75-120 nm, or about 75-100 nm. In some embodiments, an LNP composition has a diameter of 10-150 nm or a population of LNPs has an average diameter of 10-150 nm. In some embodiments, an LNP composition has a diameter of 75-150 nm or a population of LNPs has an average diameter of 75-150 nm.

LNPs are formed by precise mixing of a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are or include LNPs. As used herein, a “lipid nucleic acid assembly” comprises a plurality of (i.e., more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. In some embodiments, the aqueous nucleic acid solution comprises RNA, such as an mRNA or a gRNA. In some embodiments, the aqueous nucleic acid solution comprises an mRNA encoding a polypeptide, e.g., an RNA-guided DNA binding agent, such as Cas9.

In some embodiments, the lipid nucleic acid assembly formulations include an amine lipid (sometimes herein or elsewhere described as an “ionizable lipid” or a “biodegradable lipid”), together with an optional helper lipid, a neutral lipid, and a stealth lipid such as a PEG lipid. The terms amine lipid, helper lipid, neutral lipid, and stealth lipid have meanings known to those of skill in the art, including without limitation as set forth in International Application Publication Nos. WO2021/222287A1, WO2022/221695A1, and WO2022/221697A1, which are herein incorporated by reference with respect to the meanings of these terms and in its entirety. In some embodiments, the amine lipids or ionizable lipids are cationic depending on the pH.

The lipid nucleic acid assembly may comprise, in some embodiments, (i) an amine lipid (sometimes herein or elsewhere described as an “ionizable lipid” or a “biodegradable lipid”, (ii) an optional neutral lipid, (iii) an optional helper lipid, and (iv) a stealth lipid, such as a PEG lipid. The lipid nucleic acid assembly may comprise, in some embodiments, an amine lipid and one or more of a neutral lipid, a helper lipid, and a stealth lipid, such as a PEG lipid. The lipid nucleic acid assembly may comprise, in some embodiments, (i) an amine lipid, (ii) a neutral lipid, (iii) a helper lipid, and (iv) a stealth lipid, e.g., a PEG lipid.

In some embodiments, lipid nucleic acid assembly compositions comprise an “amine lipid”, which is, for example an ionizable lipid such as Lipid A, or Lipid D or their equivalents, including acetal analogs of Lipid A or Lipid D.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

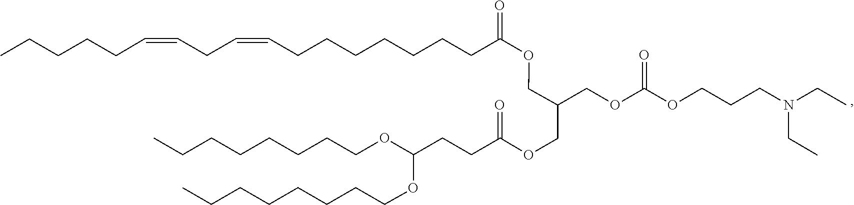 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is Lipid A, or an amine lipid provided in WO2020/219876, which is hereby incorporated by reference.

In some embodiments, an amine lipid is an analog of Lipid A. In some embodiments, a Lipid A analog is an acetal analog of Lipid A. In particular lipid nucleic acid assembly compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

In some embodiments, the amine lipid is a compound having a structure of Formula IA

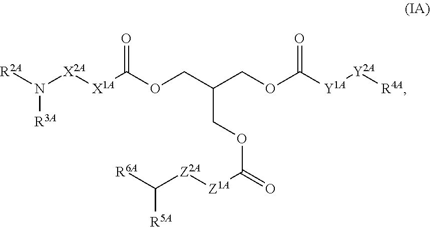

wherein
X1A is O, NH, or a direct bond;
X2A is C2-3 alkylene;
R3A is C1-3 alkyl;
R2A is C1-3 alkyl, or
R2A taken together with the nitrogen atom to which it is attached and 2-3 carbon atoms of X2A form a 5- or 6-membered ring, or
R2A taken together with R3A and the nitrogen atom to which they are attached form a 5-membered ring;
Y1A is C6-10 alkylene;
Y2A is selected from

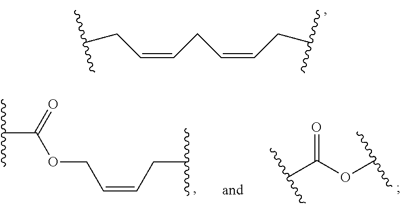 [SMILES: *C/C=C\C/C=C\C* ; *C/C=C\COC(*)=O ; *OC(*)=O]

R4A is C4-11 alkyl;
Z1A is C2-5 alkylene;
Z2A is

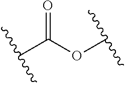 [SMILES: *OC(*)=O]

or absent;
R5A is C6-8 alkyl or C6-8 alkoxy; and
R6A is C6-8 alkyl or C6-8 alkoxy
or a salt thereof.

In some embodiments, the amine lipid is a compound of Formula (IIA)

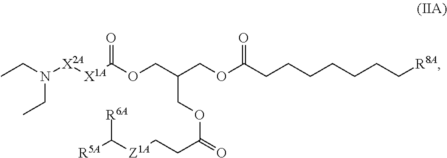

wherein
X1A is O, NH, or a direct bond;
X2A is C2-3 alkylene;
Z1A is C3 alkylene and R5A and R6A are each C6 alkyl, or Z1A is a direct bond and R5A and R6A are each C8 alkoxy; and
R8A is

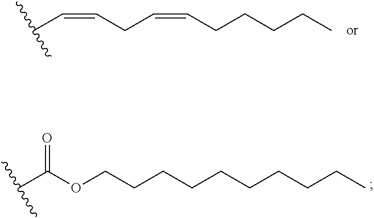 [SMILES: */C=C\C/C=C\CCCCC ; *C(=O)OCCCCCCCCCC]

or a salt thereof.

In certain embodiments, X1A is O. In other embodiments, X1A is NH. In still other embodiments, X1A is a direct bond.

In certain embodiments, X2A is C3 alkylene. In particular embodiments, X2A is C2 alkylene.

In certain embodiments, Z1A is a direct bond and R5A and R6A are each C8 alkoxy. In other embodiments, Z1A is C3 alkylene and R5A and R6A are each C6 alkyl.

In certain embodiments, R8A is

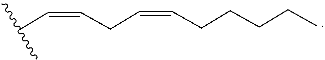 [SMILES: */C=C\C/C=C\CCCCC]

In other embodiments, R8A is

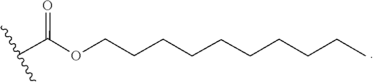 [SMILES: *C(=O)OCCCCCCCCCC]

In certain embodiments, the amine lipid is a salt.

Representative compounds of Formula (IA) include:

| Com- | |
|---|---|
| pound | |
| Number | Compound |
| 1A | 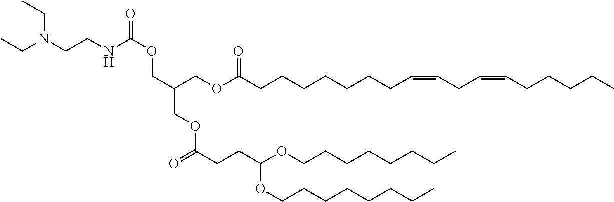 [SMILES: CCCCC/C=C\C/C=C\CCCCCCCC(=O)OCC(COC(=O)CCC(OCCCCCCCC)OCCCCCCCC)COC(=O)NCCN(CC)CC] |
| 2A | 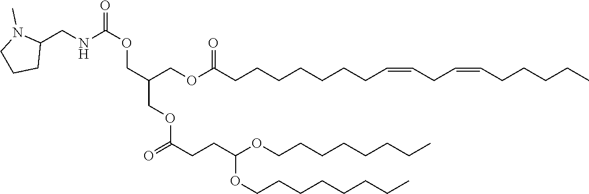 [SMILES: CCCCC/C=C\C/C=C\CCCCCCCC(=O)OCC(COC(=O)CCC(OCCCCCCCC)OCCCCCCCC)COC(=O)NCC1CCCN1C] |
| 3A | 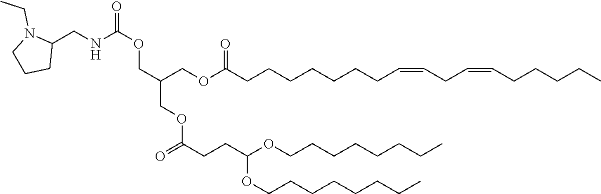 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 4A | 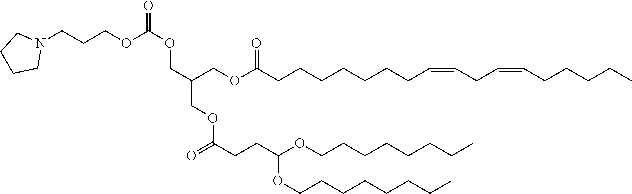 [SMILES: CCCCC/C=C\C/C=C\CCCCCCCC(=O)OCC(COC(=O)CCC(OCCCCCCCC)OCCCCCCCC)COC(=O)OCCCN1CCCC1] |
| 5A | 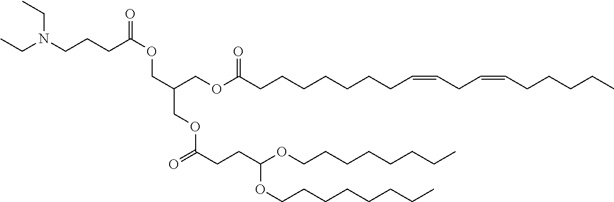 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 6A | 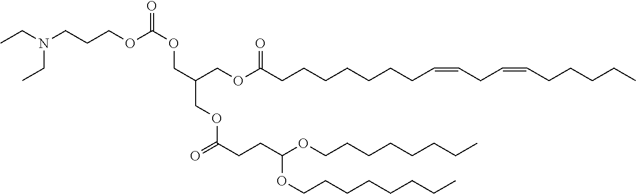 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 7A | 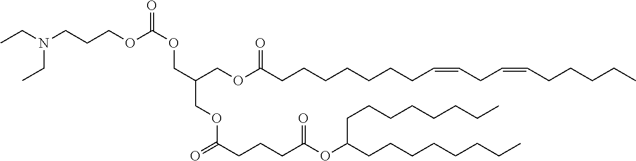 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC] |
| 8A | 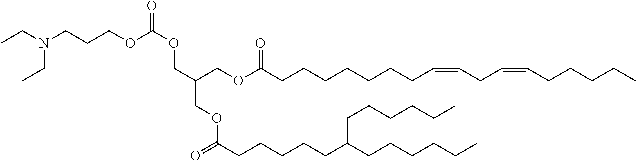 [SMILES: CCCCC/C=C\C/C=C\CCCCCCCC(=O)OCC(COC(=O)CCCCCC(CCCCCC)CCCCCC)COC(=O)OCCCN(CC)CC] |
| 9A | 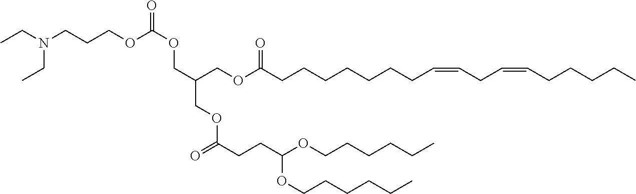 [SMILES: CCCCC/C=C\C/C=C\CCCCCCCC(=O)OCC(COC(=O)CCC(OCCCCCC)OCCCCCC)COC(=O)OCCCN(CC)CC] |
| 10A | 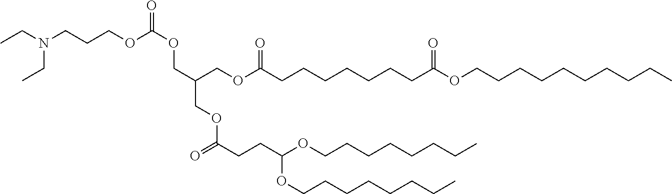 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 11A | 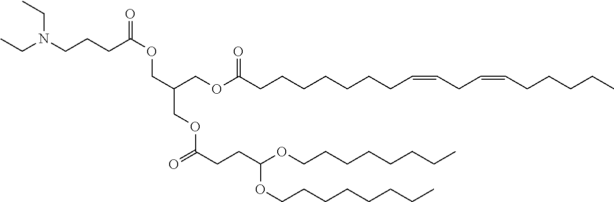 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 12A | 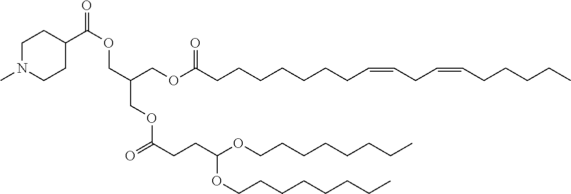 [SMILES: CCCCC/C=C\C/C=C\CCCCCCCC(=O)OCC(COC(=O)CCC(OCCCCCCCC)OCCCCCCCC)COC(=O)C1CCN(C)CC1] |
| 13A | 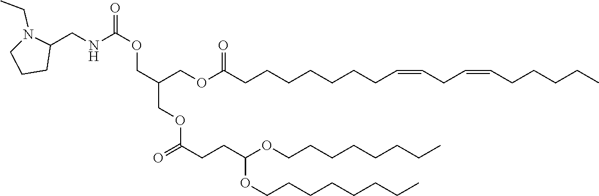 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 14A | 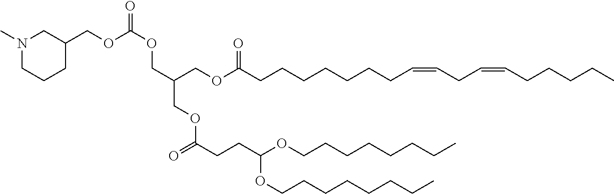 [SMILES: CCCCC/C=C\C/C=C\CCCCCCCC(=O)OCC(COC(=O)CCC(OCCCCCCCC)OCCCCCCCC)COC(=O)OCC1CCCN(C)C1] |
| 15A | 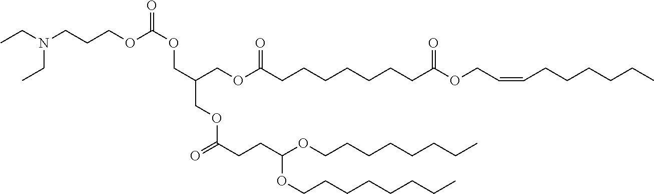 [SMILES: CCCCCC/C=C\COC(=O)CCCCCCCC(=O)OCC(COC(=O)CCC(OCCCCCCCC)OCCCCCCCC)COC(=O)OCCCN(CC)CC] |
| 16A | 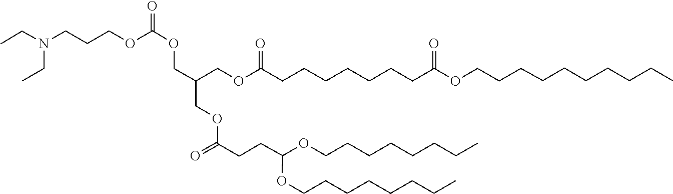 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 17A | 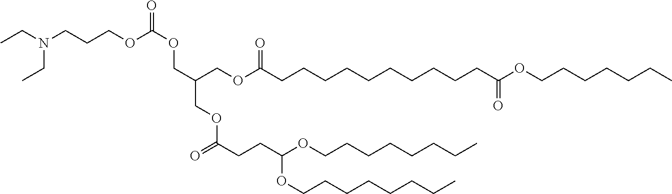 [SMILES: CCCCCCCCOC(CCC(=O)OCC(COC(=O)CCCCCCCCCCC(=O)OCCCCCCC)COC(=O)OCCCN(CC)CC)OCCCCCCCC] |
| 18A | 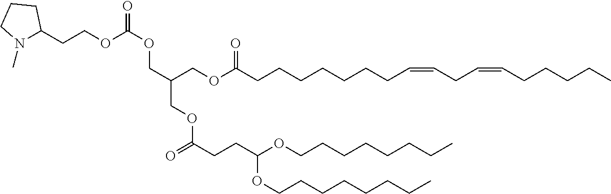 [SMILES: CCCCC/C=C\C/C=C\CCCCCCCC(=O)OCC(COC(=O)CCC(OCCCCCCCC)OCCCCCCCC)COC(=O)OCCC1CCCN1C] |
| 19A | 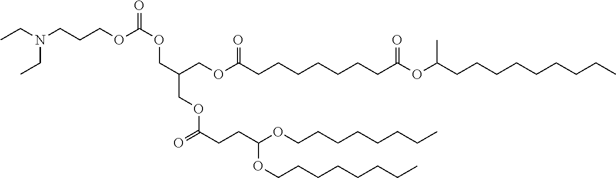 [SMILES: CCCCCCCCCC(C)OC(=O)CCCCCCCC(=O)OCC(COC(=O)CCC(OCCCCCCCC)OCCCCCCCC)COC(=O)OCCCN(CC)CC] |

or a salt thereof, such as a pharmaceutically acceptable salt thereof.

In some embodiments, the amine lipid is Lipid D, which is nonyl 8-((7,7-bis(octyloxy)heptyl)(2-hydroxyethyl)amino)octanoate:

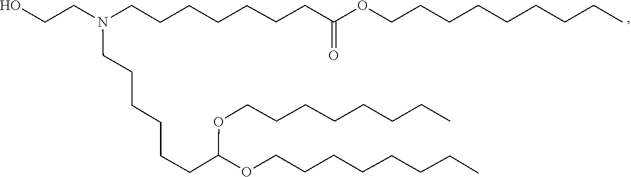 [SMILES: CCCCCCCCCOC(=O)CCCCCCCN(CCO)CCCCCCC(OCCCCCCCC)OCCCCCCCC]

or a salt thereof.

Lipid D may be synthesized according to WO2020072605 and Mol. Ther. 2018, 26(6), 1509-1519 (“Sabnis”), which are incorporated by reference in their entireties. In some embodiments, the amine lipid Lipid D, or an amine lipid provided in WO2020072605, which is hereby incorporated by reference.

In some embodiments, the amine lipid is a compound having a structure of Formula IB:

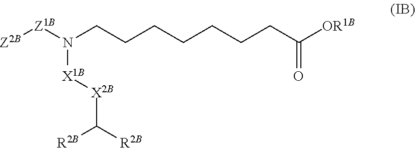

wherein
X^{1B} is C₆₋₇ alkylene;
X^{2B} is

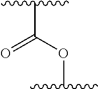 [SMILES: *OC(*)=O]

or absent, provided that if X^{2B} is

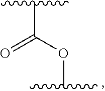 [SMILES: *OC(*)=O]

R^{2B} is not alkoxy;
Z^{1B} is C₂₋₃ alkylene;
Z^{2B} is selected from —OH, —NHC(═O)OCH₃, and —NHS(═O)₂CH₃;
R^{1B} is C₇₋₉ unbranched alkyl; and
each R^{2B} is independently C₈ alkyl or C₈ alkoxy;
or a salt thereof

In some embodiments, the amine lipid is a compound of Formula (IIB)

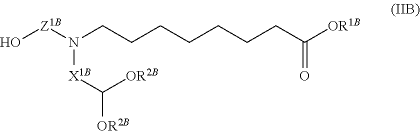

wherein
X^{1B} is C₆₋₇ alkylene;
Z^{1B} is C₂₋₃ alkylene;
R^{1B} is C₇₋₉ unbranched alkyl; and
each R^{2B} is C₈ alkyl;
or a salt thereof.

In certain embodiments, X^{1B} is C₆ alkylene. In other embodiments, X^{1B} is C₇ alkylene.

In certain embodiments, Z^{1B} is a direct bond and R^{5B} and R^{6B} are each C₈ alkoxy. In other embodiments, Z^{1B} is C₃ alkylene and R^{5B} and R^{6B} are each C₆ alkyl.

In certain embodiments, X^{2B} is

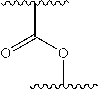 [SMILES: *OC(*)=O]

and R^{2B} is not alkoxy. In other embodiments, X^{2B} is absent.

In certain embodiments, Z^{1B} is C₂ alkylene; In other embodiments, Z^{1B} is C₃ alkylene.

In certain embodiments, Z^{2B} is —OH. In other embodiments, Z^{2B} is —NHC(═O)OCH₃. In other embodiments, Z^{2B} is —NHS(═O)₂CH₃.

In certain embodiments, R^{1B} is C₇ unbranched alkylene. In other embodiments, R^{1B} is C₈ branched or unbranched alkylene. In other embodiments, R^{1B} is C₉ branched or unbranched alkylene.

In certain embodiments, the amine lipid is a salt.

Representative compounds of Formula (IB) include:

| Compound | |
|---|---|
| Number | Compound |
| 1B | 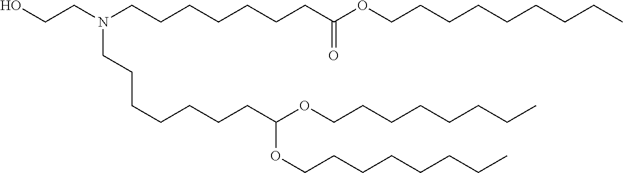 [SMILES: CCCCCCCCCOC(=O)CCCCCCCN(CCO)CCCCCCCC(OCCCCCCCC)OCCCCCCCC] |
| 2B | 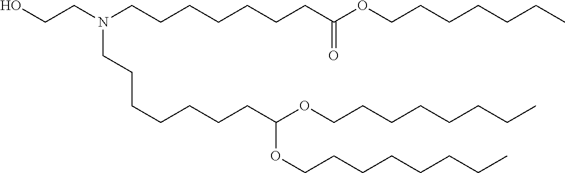 [SMILES: CCCCCCCCOC(CCCCCCCN(CCO)CCCCCCCC(=O)OCCCCCCC)OCCCCCCCC] |
| 3B | 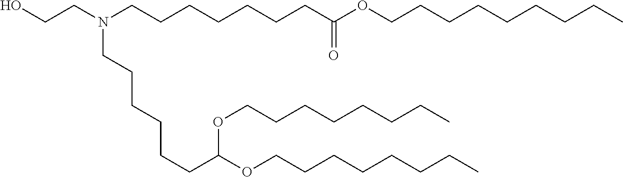 [SMILES: CCCCCCCCCOC(=O)CCCCCCCN(CCO)CCCCCCC(OCCCCCCCC)OCCCCCCCC] |
| 4B | 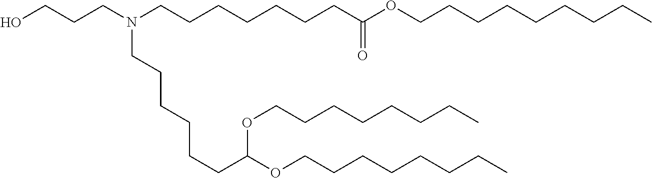 [SMILES: CCCCCCCCCOC(=O)CCCCCCCN(CCCO)CCCCCCC(OCCCCCCCC)OCCCCCCCC] |
| 5B | 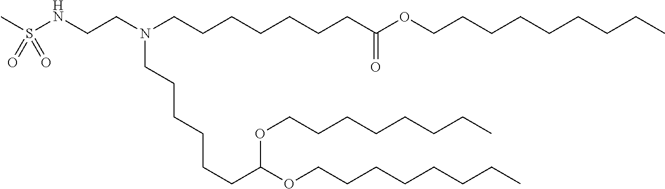 [SMILES: CCCCCCCCCOC(=O)CCCCCCCN(CCCCCCC(OCCCCCCCC)OCCCCCCCC)CCNS(C)(=O)=O] |
| 6B | 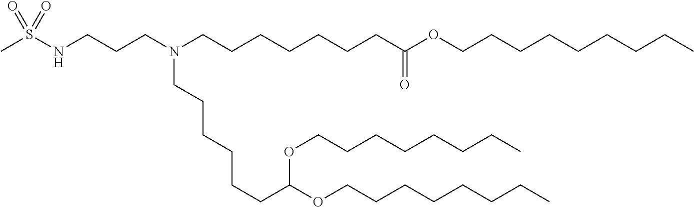 [SMILES: CCCCCCCCCOC(=O)CCCCCCCN(CCCCCCC(OCCCCCCCC)OCCCCCCCC)CCCNS(C)(=O)=O] |
| 7B | 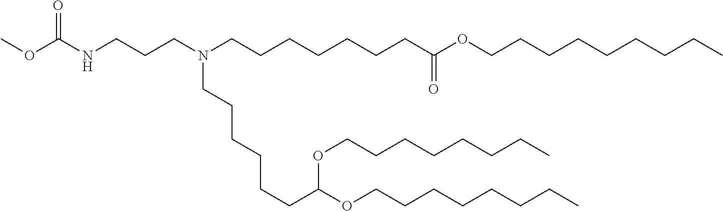 [SMILES: CCCCCCCCCOC(=O)CCCCCCCN(CCCCCCC(OCCCCCCCC)OCCCCCCCC)CCCNC(=O)OC] |

or a salt thereof, such as a pharmaceutically acceptable salt thereof.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g. an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO/2020/219876 (e.g., at pp. 13-33, 66-87), WO/2020/118041, WO/2020/072605 (e.g., at pp. 5-12, 21-29, 61-68, WO/2019/067992, WO/2017/173054, WO2015/095340, and WO2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of lipid nucleic acid assembly compositions of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (IV), wherein n is 45, meaning that the number averaged degree of polymerization comprises about 45 subunits

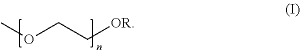

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG) (cat. #880150P from Avanti Polar Lipids, Alabaster, Alabama, USA), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In some embodiments, the LNP compositions include polymeric lipids, such as PEG lipids which can affect the length of time the nanoparticles can exist in vivo or ex vivo (e.g, in the blood or medium). PEG lipids may assist in the formulation process by, for example, reducing particle aggregation and controlling particle size. PEG lipids used herein may modulate pharmacokinetic properties of the LNPs. Typically, the PEG lipid comprises a lipid moiety and a polymer moiety based on PEG (sometimes referred to as poly(ethylene oxide)) (a PEG moiety). PEG lipids suitable for use in a lipid composition with a compound of Formula (I) or (II) of the present disclosure and information about the biochemistry of such lipids can be found in Romberg et al., Pharmaceutical Research 25(1), 2008, pp. 55-71 and Hoekstra et al., Biochimica et Biophysica Acta 1660 (2004) 41-52. Additional suitable PEG lipids are disclosed, e.g., in WO 2015/095340 (p. 31, line 14 to p. 37, line 6), WO 2006/007712, and WO 2011/076807 (“stealth lipids”), each of which is incorporated by reference in its entirety.

In preferred embodiments, the PEG lipid includes a glycerol group. In preferred embodiments, the PEG lipid includes a dimyristoylglycerol (DMG) group. In preferred embodiments, the PEG lipid comprises PEG-2k. In preferred embodiments, the PEG lipid is a PEG-DMG. In preferred embodiments, the PEG lipid is a PEG-2k-DMG. In preferred embodiments, the PEG lipid is 1,2-dimyristoyl-rac-glycero-3-methoxypolyethylene glycol-2000. In preferred embodiments, the PEG-2k-DMG is 1,2-dimyristoyl-rac-glycero-3-methoxypolyethylene glycol-2000.

In some embodiments, a lipid nucleic acid assembly composition may comprise the RNA component, an amine lipid, a helper lipid, a neutral lipid, and a stealth lipid. In certain lipid nucleic acid assembly compositions, the helper lipid is cholesterol. In other compositions, the neutral lipid is DSPC. In additional embodiments, the stealth lipid is a PEG2k-DMG.

In some embodiments, lipid compositions are described according to the respective molar ratios of the component lipids in the lipid portion of the formulation. In one embodiment, the mol % of the amine lipid may be from about 30 mol % to about 60 mol %. In one embodiment, the mol % of the amine lipid may be from about 40 mol % to about 60 mol %. In one embodiment, the mol % of the amine lipid may be from about 45 mol % to about 60 mol %. In one embodiment, the mol % of the amine lipid may be from about 50 mol % to about 60 mol %. In one embodiment, the mol % of the amine lipid may be from about 55 mol % to about 60 mol %. In one embodiment, the mol % of the amine lipid may be from about 50 mol % to about 55 mol %. In one embodiment, the mol % of the amine lipid may be about 50 mol %. In one embodiment, the mol % of the amine lipid may be about 55 mol %. In some embodiments, the amine lipid mol % of the lipid nucleic acid assembly batch will be ±30%, ±25%, ±20%, ±15%, ±10%, ±5%, or ±2.5% of the target mol %. In some embodiments, the amine lipid mol % of the lipid nucleic acid assembly batch will be ±4 mol %, ±3 mol %, ±2 mol %, ±1.5 mol %, ±1 mol %, ±0.5 mol %, or ±0.25 mol % of the target mol %. All mol % numbers are given as a fraction of the lipid component of the lipid nucleic acid assembly compositions. In some embodiments, lipid nucleic acid assembly inter-lot variability of the amine lipid mol % will be less than 15%, less than 10% or less than 5%.

In one embodiment, the mol % of the neutral lipid may be from about 5 mol % to about 15 mol %. In one embodiment, the mol % of the neutral lipid may be from about 7 mol % to about 12 mol %. In one embodiment, the mol % of the neutral lipid may be about 9 mol %. In some embodiments, the neutral lipid mol % of the lipid nucleic acid assembly batch will be ±30%, ±25%, ±20%, ±15%, ±10%, ±5%, or ±2.5% of the target neutral lipid mol %. In some embodiments, lipid nucleic acid assembly inter-lot variability will be less than 15%, less than 10% or less than 5%.

In one embodiment, the mol % of the helper lipid may be from about 20 mol % to about 60 mol %. In one embodiment, the mol % of the helper lipid may be from about 25 mol % to about 55 mol %. In one embodiment, the mol % of the helper lipid may be from about 25 mol % to about 50 mol %. In one embodiment, the mol % of the helper lipid may be from about 25 mol % to about 40 mol %. In one embodiment, the mol % of the helper lipid may be from about 30 mol % to about 50 mol %. In one embodiment, the mol % of the helper lipid may be from about 30 mol % to about 40 mol %. In one embodiment, the mol % of the helper lipid is adjusted based on amine lipid, neutral lipid, and PEG lipid concentrations to bring the lipid component to 100 mol %. In some embodiments, the helper mol % of the lipid nucleic acid assembly batch will be ±30%, ±25%, ±20%, ±15%, ±10%, ±5%, or ±2.5% of the target mol %. In some embodiments, lipid nucleic acid assembly inter-lot variability will be less than 15%, less than 10% or less than 5%.

In one embodiment, the mol % of the PEG lipid may be from about 1 mol % to about 10 mol %. In one embodiment, the mol % of the PEG lipid may be from about 2 mol % to about 10 mol %. In one embodiment, the mol % of the PEG lipid may be from about 1 mol % to about 3 mol %. In one embodiment, the mol % of the PEG lipid may be from about 2 mol % to about 4 mol %. In one embodiment, the mol % of the PEG lipid may be from about 1.5 mol % to about 2 mol %. In one embodiment, the mol % of the PEG lipid may be from about 2.5 mol % to about 4 mol %. In one embodiment, the mol % of the PEG lipid may be about 3 mol %. In one embodiment, the mol % of the PEG lipid may be about 2.5 mol %. In one embodiment, the mol % of the PEG lipid may be about 2 mol %. In one embodiment, the mol % of the PEG lipid may be about 1.5 mol %. In some embodiments, the PEG lipid mol % of the lipid nucleic acid assembly batch will be ±30%, ±25%, ±20%, ±15%, ±10%, ±5%, or ±2.5% of the target PEG lipid mol %. In some embodiments, lipid nucleic acid assembly composition, e.g. the LNP composition, inter-lot variability will be less than 15%, less than 10% or less than 5%.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, a lipid nucleic acid assembly composition may comprise a lipid component that comprises an amine lipid, a helper lipid, a neutral lipid, and a helper lipid; and a nucleic acid component, wherein the N/P ratio is about 3 to 10. In some embodiments, the LNPs comprise molar ratios of an amine lipid to RNA/DNA phosphate (N:P) of about 4.5, 5.0, 5.5, 6.0, or 6.5. In some embodiments, a lipid nucleic acid assembly composition may comprise a lipid component that comprises an amine lipid, a helper lipid, a neutral lipid, and a helper lipid; and an RNA component, wherein the N/P ratio is about 3 to 10. In one embodiment, the N/P ratio may about 5-7. In one embodiment, the N/P ratio may about 4.5-8. In one embodiment, the N/P ratio may about 6. In one embodiment, the N/P ratio may be 6±1. In one embodiment, the N/P ratio may about 6±0.5. In some embodiments, the N/P ratio will be ±30%, ±25%, ±20%, ±15%, ±10%, ±5%, or 2.5% of the target N/P ratio. In some embodiments, lipid nucleic acid assembly inter-lot variability will be less than 15%, less than 10% or less than 5%.

In some embodiments, a lipid nucleic acid assembly composition comprises lipid nanoparticles (LNPs). In some embodiments, two or more nucleic acids in a lipid nucleic acid assembly composition can be encapsulated in LNPs. LNPs can include cationic lipids together with other components such as neutral phospholipids, phosphatidylcholines, sterols such as cholesterol, and/or PEGylated phospholipids. Solid lipid nanoparticles (SLNs), produced using lipids that are solid at room temperature and at body temperature, are colloidal nanoparticles with a solid lipophilic core. The solid lipid core of an SLN can include triglycerides (e.g., tri-stearin), glyceride mixtures or partial glycerides (e.g., Imwitor), fatty acids (e.g., stearic acid or palmitic acid), steroids (e.g., cholesterol), and/or waxes (e.g., cetyl palmitate) that are solid at both room temperature and human body temperature. Lipid nano-emulsions (LNE) are colloidal nanoparticles with a core that is liquid at room temperature. Nanostructured lipid carriers (NLCs) include a mixture of solid and liquid lipids, such as glyceryl tricaprylate, ethyl oleate, isopropyl myristate, and/or glyceryl dioleate.

In some embodiments, the lipid nucleic acid assembly compositions include a Cas nuclease mRNA, such as a Class 2 Cas mRNA, and at least one gRNA. In some embodiments, the lipid nucleic acid assembly composition includes a ratio of gRNA to Cas nuclease mRNA, such as Class 2 Cas nuclease mRNA from about 25:1 to about 1:25 wt/wt. In some embodiments, the lipid nucleic acid assembly formulation includes a ratio of gRNA to Cas nuclease mRNA, such as Class 2 Cas nuclease mRNA from about 10:1 to about 1:10. In some embodiments, the lipid nucleic acid assembly formulation includes a ratio of gRNA to Cas nuclease mRNA, such as Class 2 Cas nuclease mRNA from about 8:1 to about 1:8. As measured herein, the ratios are by weight. In some embodiments, the lipid nucleic acid assembly formulation includes a ratio of gRNA to Cas nuclease mRNA, such as Class 2 Cas mRNA from about 5:1 to about 1:5. In some embodiments, ratio range is about 3:1 to 1:3, about 2:1 to 1:2, about 5:1 to 1:2, about 5:1 to 1:1, about 3:1 to 1:2, about 3:1 to 1:1, about 3:1, about 2:1 to 1:1. In some embodiments, the gRNA to mRNA ratio is about 3:1 or about 2:1. In some embodiments the ratio of gRNA to Cas nuclease mRNA, such as Class 2 Cas nuclease is about 1:1. In some embodiments the ratio of gRNA to Cas nuclease mRNA, such as Class 2 Cas nuclease is about 1:2. The other embodiments, the ratio may be about 25:1, 10:1, 5:1, 3:1, 2:1, 1:1, 1:2, 1:3, 1:5, 1:10, or 1:25.

The lipid nucleic acid assembly compositions disclosed herein may include a template nucleic acid. The template nucleic acid may be co-formulated with an mRNA encoding a polypeptide such as a Cas nuclease, such as a Class 2 Cas nuclease mRNA. In some embodiments, the template nucleic acid may be co-formulated with a guide RNA. In some embodiments, the template nucleic acid may be co-formulated with both an mRNA encoding a polypeptide such as a Cas nuclease and a guide RNA. In some embodiments, the template nucleic acid may be formulated separately from an mRNA encoding a polypeptide such as a Cas nuclease or a guide RNA. The template nucleic acid may be delivered with, or separately from the lipid nucleic acid assembly compositions. In some embodiments, the template nucleic acid may be single- or double-stranded, depending on the desired repair mechanism. The template may have regions of homology to the target DNA, or to sequences adjacent to the target DNA.

In some embodiments, the lipid nucleic acid assembly compositions include a nucleic acid encoding a DNA base editor. In particular embodiments, a base editing enzyme includes a cytidine deaminase domain or an adenine deaminase domain. Base editors including deaminases that deaminate cytosine can be referred to as cytosine base editors. Base editors including deaminases that deaminate adenosine can be referred to as adenosine base editors.

In some embodiments, the lipid nucleic acid assembly composition can additionally include an mRNA encoding a DNA glycosylase inhibitor. In some embodiments, the DNA glycosylase inhibitor is a uracil glycosylase inhibitor (UGI).

In certain exemplary embodiments, a lipid nucleic acid assembly composition can include a gRNA (e.g., a gRNA of about 80 to about 120 nucleotides or about 80 to about 130 nucleotides in length), a first mRNA encoding a DNA glycosylase inhibitor such as UGI (e.g., an mRNA of about 1000 nucleotides in length), and a second mRNA encoding a cytosine base editor (e.g., an mRNA of about 4500 nucleotides in length).

In some embodiments, lipid nucleic acid assemblies are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of lipid nucleic acid assemblies, may be used. In some embodiments, a buffer is used to maintain the pH of the composition comprising lipid nucleic acid assemblies at or above pH 6.5. In some embodiments, a buffer is used to maintain the pH of the composition comprising lipid nucleic acid assemblies at or above pH 7.0. In some embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In some embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In some embodiments, the lipid nucleic acid assembly composition may include about 1, 2, 3, 4, 5, 6, 7, 8, 9, or 10% cryoprotectant. In some embodiments, the lipid nucleic acid assembly composition may include about 1, 2, 3, 4, 5, 6, 7, 8, 9, or 10% sucrose. In some embodiments, the lipid nucleic acid assembly composition may include a buffer. In some embodiments, the buffer may comprise a phosphate buffer (PBS), a Tris buffer, a citrate buffer, and mixtures thereof. In some exemplary embodiments, the buffer comprises NaCl. In some embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the lipid nucleic acid assembly compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used to prepare the lipid nucleic acid assembly compositions. In certain aspects, flow rates, junction size, junction geometry, junction shape, tube diameter, solutions, and/or RNA and lipid concentrations may be varied. Lipid nucleic acid assemblies or lipid nucleic acid assembly compositions may be concentrated or purified, e.g., via dialysis, tangential flow filtration, or chromatography. The lipid nucleic acid assemblies may be stored as a suspension, an emulsion, or a lyophilized powder, for example. In some embodiments, a lipid nucleic acid assembly composition is stored at 2-8° C., in certain aspects, the lipid nucleic acid assembly compositions are stored at room temperature. In additional embodiments, a lipid nucleic acid assembly composition is stored frozen, for example at −20° C. or −80° C. In other embodiments, a lipid nucleic acid assembly composition is stored at a temperature ranging from about 0° C. to about −80° C. Frozen lipid nucleic acid assembly compositions may be thawed before use, for example on ice, at 4° C., at room temperature, or at 25° C. Frozen lipid nucleic acid assembly compositions may be maintained at various temperatures, for example on ice, at 4° C., at room temperature, at 25° C., or at 37° C.

In some embodiments, the lipid nucleic acid assembly composition comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the LNP composition is about 3-10. In some embodiments, the lipid nucleic acid assembly composition comprises: about 50-60 mol-% amine lipid; about 8-10 mol-% neutral lipid; and about 2.5-4 mol-% PEG lipid, wherein the remainder of the lipid component is helper lipid, and wherein the N/P ratio of the LNP composition is about 3-8. In some embodiments, the lipid nucleic acid assembly composition comprises: about 50-60 mol-% amine lipid; about 5-15 mol-% DSPC; and about 2.5-4 mol-% PEG lipid, wherein the remainder of the lipid component is cholesterol, and wherein the N/P ratio of the LNP composition is 3-8±0.2.

In embodiments, the average diameter is a Z-average diameter. In certain embodiments, the Z-average diameter is measured by dynamic light scattering (DLS) using methods known in the art. For example, average particle size and polydispersity can be measured by dynamic light scattering (DLS) using a Malvern Zetasizer DLS instrument. LNP samples are diluted with PBS buffer prior to being measured by DLS. Z-average diameter and number average diameter along with a polydispersity index (pdi) can be determined. The Z average is the intensity weighted mean hydrodynamic size of the ensemble collection of particles. The number average is the particle number weighted mean hydrodynamic size of the ensemble collection of particles. A Malvern Zetasizer instrument can also be used to measure the zeta potential of the LNP using methods known in the art.

Lipid Nucleic Acid Assembly Compositions that Include Nucleic Acids

The present disclosure provides, among other things, compositions and methods for assessment of lipid nucleic acid assembly compositions that include nucleic acids (e.g., methods that can include one or more, or all, of separating, quantifying, and/or characterizing nucleic acids present in a lipid nucleic acid assembly composition). Various methods of lipid nucleic acid assembly composition assessment provided herein can be used to separate, quantify, and/or characterize nucleic acids present in wide variety of lipid nucleic acid assembly compositions, examples of which can include drug substances and drug products.

In some embodiments, a lipid nucleic acid assembly composition can include ribonucleic acid (RNA) molecules. In some embodiments, an RNA molecule can include a messenger RNA (mRNA) molecule or guide RNA (gRNA). In various embodiments, nucleic acid present in a lipid nucleic acid assembly composition can consist or consist essentially of mRNA and gRNA (e.g., a single mRNA and a single gRNA, or two mRNAs and a single gRNA).

In some embodiments, an mRNA molecule can encode a polypeptide, such as an RNA-guided DNA binding agent. In some embodiments, an RNA-guided DNA binding agent is a naturally occurring polypeptide. In some embodiments, an RNA-guided DNA binding agent is a variant of a naturally occurring polypeptide (e.g., a polypeptide having the sequence of a naturally-occurring polypeptide that is modified, e.g., by mutation, deletion, and/or insertion).

In some embodiments, an mRNA molecule can encode a nuclease (e.g., a nuclease of a gene editing system). In some embodiments, an mRNA molecule can encode a clustered regularly interspaced short palindromic repeats (CRISPR)-associated (Cas) nuclease (e.g., a CRISPR-associated RNA-guided endonuclease).

Cas nucleases include nucleases of three types (designated as type I, type II, and type III), and 10 subtypes including 5 type I, 3 type II, and 2 type III proteins (see, e.g., Hochstrasser and Doudna, Trends Biochem Sci, 2015:40(1):58-66). Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, CasY, Cpf1, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx100, Csx16, CsaX, Csx3, Csx1, Csx15, Csf1, Csf2, Csf3, Csf4, and variants thereof. These nucleases are known to those skilled in the art.

In particular embodiments, Cas9 refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, includes one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species. In various embodiments, variants of the Cas9 nuclease include a single inactive catalytic domain, such as a RuvC″ or HNH″ enzyme or a nickase. A Cas9 nickase has only one active functional domain and, in some embodiments, cuts only one strand of the target DNA, thereby creating a single strand break or nick. Exemplary Cas9 amino acid sequence include a Streptococcus pyogenes wild-type Cas9 polypeptide as set forth, e.g., in NCBI accession no. NP 269215, and a Streptococcus thermophilus wild-type Cas9 polypeptide as set forth, e.g., in NCBI accession no. WP_011681470.

In some embodiments, an mRNA molecule can encode a fusion polypeptide. In some embodiments, an mRNA molecule can encode a fusion polypeptide that is an RNA-guided DNA binding agent that includes a heterologous polypeptide. In some embodiments, an mRNA molecule can encode a fusion polypeptide that includes an RNA-guided endonuclease and a heterologous polypeptide. Various such fusion polypeptides, including site-directed polypeptides, are disclosed in WO 2013/176772, which is incorporated herein by reference with respect to fusion partners and fusion polypeptides, and in its entirety.

In various embodiments, an mRNA molecule can encode a fusion polypeptide that is a site-directed polypeptide that includes two portions, an RNA-binding portion and an activity portion. In some embodiments, a site-directed polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA. In some embodiments the RNA binding portion (i) also has activity, such as nickase activity.

In some embodiments, a site-directed polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that modulates transcription within the target DNA (e.g., to increase or decrease transcription), wherein the site of modulated transcription within the target DNA is determined by the DNA-targeting RNA.

In some embodiments, a site-directed polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In some embodiments, a site-directed polypeptide can have an enzymatic activity that modifies a polypeptide (e.g., a histone) associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some embodiments, a heterologous sequence of a fusion polypeptide can provide for subcellular localization of a site-directed modifying polypeptide (e.g., a nuclear localization signal (NLS) for targeting to the nucleus; a mitochondrial localization signal for targeting to the mitochondria; a chloroplast localization signal for targeting to a chloroplast; a ER retention signal; and the like).

In some embodiments, a heterologous sequence of a fusion polypeptide can provide (e.g., directly provide) for increased transcription of the target nucleic acid (e.g., a transcription activator or a fragment thereof, a protein or fragment thereof that recruits a transcription activator, a small molecule/drug-responsive transcription regulator, etc.).

In some embodiments, an mRNA can encode a base editor including a deaminase associated with a DNA binding domain such as a catalytically impaired nuclease domain (e.g., a nickase, e.g., catalytically impaired CRISPR/Cas). Exemplary Cas nucleases are provided herein. Particular embodiments utilize a nuclease-inactive Cas9 (dCas9) as the catalytically disabled nuclease. For example, in various embodiments, a cytosine base editor can include Nme2Cas9(D16A) nickase fused to APOBEC3A deaminase, and in various embodiments does not include a DNA glycosylase inhibitor. However, any nuclease of the CRISPR system can be engineered to produce a catalytically impaired nuclease domain (e.g., a nickase) for use in a base editor including the engineered nuclease and a deaminase.

Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); and Carrington et al., Cells 9:1690 (2020)). Additional information regarding deaminases and base editors is found throughout PCT Publication No. WO 2022/125968, which is incorporated herein by reference in its entirety.

In some embodiments, the cytidine deaminase disclosed herein is an enzyme of APOBEC family. In some embodiments, the cytidine deaminase disclosed herein is an enzyme of APOBEC 1, APOBEC2, APOBEC4, and APOBEC3 subgroups. In some embodiments, the cytidine deaminase disclosed herein is an enzyme of APOBEC3 subgroup. In some embodiments, the cytidine deaminase disclosed herein is an APOBEC3A deaminase (A3A).

In some embodiments, an APOBEC3A deaminase (A3A) disclosed herein is a human A3A. In some embodiments, the A3A is a wild-type A3A. In some embodiments, the wild-type A3A is a human A3A (UniPROT accession ID: p319411). In some embodiment, the A3A is an A3A variant. A3A variants share homology to wild-type A3A, or a fragment thereof. In some embodiments, a A3A variant has at least about 80% identity, at least about 85% identity, at least about 90% identity, at least about 95% identity, at least about 96% identity, at least about 97% identity, at least about 98% identity, at least about 99% identity, at least about 99.5% identity, or at least about 99.9% identity to a wild type A3 Å. In some embodiments, the A3A variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type A3A.

Exemplary adenosine deaminases can include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. In some embodiments, an ABE can include a wild-type E. coli tRNA-specific adenosine deaminase (TadA) monomer and a TadA* mutant TadA monomer that catalyzes deoxyadenosine deamination. In certain embodiments, there is a linker positioned between TadA and TadA*, and in certain embodiments there is a linker positioned between TadA* and the DNA binding domain (e.g., a Cas nickase).

Further non-limiting examples of base editors include BE1 (APOBEC1-16 amino acid (aa) linker-Sp dCas9 (D10A, H840A) (see, e.g., Komor 2016 Nature 533: 420-424)), BE2 (APOBEC1-16aa linker-Sp dCas9 (D10A, H840A)-4aa linker-UGI (see, e.g., Komor 2016 Nature 533: 420-424)), BE3 (APOBEC1-16aa linker-SpnCas9 (D10A)-4aa linker-UGI (see, e.g., Komor 2016 Nature 533: 420-424)), HIF-BE2 (rAPOBEC1-HF2 nCas9-UGI), HIF-BE3 (APOBEC1-16aa linker-HF nCas9 (D10A)-4aa linker-UGI (see, e.g., Rees 2017 Nat. Commun. 8: 15790)), BE4 (rAPOBEC1-Sp nCas9-UGI-UGI), BE4max (APOBEC1-32aa linker-Sp nCas9 (D10A)-9aa linker-UGI-9aa linker-UGI (see, e.g., Koblan 2018 Nat. Biotechnol 36(9): 843-846 and/or Komor 2017 Sci. Adv. 3(8): eaao4774)), BE4-GAM (Gam-16aa linker-APOBEC1-32aa linker-Sp nCas9 (D10A)-9aa linker-UGI-9aa linker-UGI (see, e.g., Komor 2017 Sci. Adv. 3(8): eaao4774)), YE1-BE3 (APOBEC1 (W90Y, R126E)-16aa linker-Sp nCas9 (D10A)-4aa linker-UGI (see, e.g., Kim 2017 Nat. Biotechnol. 35: 475-480)), EE-BE3 (APOBEC1 (R126E, R132E)-16aa linker-Sp nCas9 (D10A)-4aa linker-UGI (see, e.g., Kim 2017 Nat. Biotechnol. 35: 475-480)), YE2-BE3 (APOBEC1 (W90Y, R132E)-16aa linker-Sp nCas9 (D10A)-4aa linker-UGI (see, e.g., Kim 2017 Nat. Biotechnol. 35: 475-480)), YEE-BE3 (APOBEC1 (W90Y, R126E, R132E)-16aa linker-Sp nCas9 (D10A)-4aa linker-UGI (see, e.g., Kim 2017 Nat. Biotechnol. 35: 475-480)), VQR-BE3 (APOBEC1-16aa linker-Sp VQR nCas9 (D10A)-4aa linker-UGI (see, e.g., Kim 2017 Nat. Biotechnol. 35: 475-480)), EQR-BE3 (rAPOBEC1-EQR SpnCas9-UGI), VRER-BE3 (APOBEC1-16aa linker-Sp VRER nCas9 (D10A)-4aa linker-UGI (see, e.g., Kim 2017 Nat. Biotechnol. 35: 475-480)), Sa-BE3 (APOBEC1-16aa linker-Sa nCas9 (D10A)-4aa linker-UGI (see, e.g., Kim 2017 Nat. Biotechnol. 35: 475-480)), SA-BE4 (APOBEC1-32aa linker-Sa nCas9 (D10A)-9aa linker-UGI-9aa linker-UGI (see, e.g., Komor 2017 Sci. Adv. 3(8): eaao4774)), SaBE4-Gam (Gam-16aa linker-APOBEC1-32aa linker-Sa nCas9 (D10A)-9aa linker-UGI-9aa linker-UGI (see, e.g., Komor 2017 Sci. Adv. 3(8): eaao4774)), SaKKH-BE3 (APOBEC1-16aa linker-Sa KKH nCas9 (D10A)-4aa linker-UGI (see, e.g., Kim 2017 Nat. Biotechnol. 35: 475-480)), FNLS-BE3 (rAPOBEC1-Sp nCas9-UGI), RA-BE3 (rAPOBEC1 (RA)-Sp nCas9-UGI), Cas12a-BE (APOBEC1-16aa linker-dCas12a-14aa linker-UGI (see, e.g., Li 2018 Nat. Biotechnol. 36: 324-327)), Target-AID (Sp nCas9 (D10A)-100aa linker-CDA1-9aa linker-UGI (see, e.g., Nishida 2016 Science 353(6305): aaf8729)), Target-AID-NG (Sp nCas9 (D10A)-NG-100aa linker-CDA1-9aa linker-UGI (see, e.g., Nishimasu 2018 Science 361(6408): 1259-1262)), xBE3 (APOBEC1-16aa linker-xCas9(D10A)-4aa linker-UGI (see, e.g., Hu 2018 Nature 556: 57-63)), eA3A-BE3 (APOBEC3A (N37G)-16aa linker-Sp nCas9(D10A)-4aa linker-UGI (see, e.g., Gehrke 2018 Nat. Biotechnol. 36(10): 977-982)), A3A-BE3 (hAPOBEC3A-16aa linker-Sp nCas9(D10A)-4aa linker-UGI (see, e.g., Wang 2018 Nat. Biotechnol. 36: 946-949)), eA3 Å-HF1-BE3-2xUGI (APOBEC3A-HF1 Sp nCas9-UGI-UGI), eA3A-HypaBE3-2xUGI (APOBEC3A-Hypa Sp nCas9-UGI-UGI), hA3A-BE3 (hAPOBEC3A-Sp nCas9-UGI), hA3B-BE3 (hAPOBEC3B-Sp nCas9-UGI), hA3G-BE3 (hAPOBEC3G-Sp nCas9-UGI), hAID-BE3 (hAPOBEC3A-Sp nCas9-UGI), SaCas9-BE3 (rAPOBEC1-SanCas9-UGI), xCas9-BE3 (rAPOBEC1-xnCas9-UGI), ScCas9-BE3 (rAPOBEC1-ScnCas9-UGI), SniperCas9-BE3 (rAPOBEC1-SnipernCas9-UGI), iSpyMac-BE3 (rAPOBEC1-iSpyMacnCas9-UGI), CRISPR-X (Sp dCas9-MS2-hAID), TAM (Sp dCas9-hAID (P182X)), AncBE4-Max (rAPOBEC1-Sp nCas9-UGI-UGI), ABE7.8/9/10 (ecTadA-ecTadA*-Sp nCas9), xCas9-ABE7.10 (ecTadA-ecTadA*-nxCas9), VQR-ABE (ecTadA-ecTadA*-Sp VQR nCas9), Sa(KKH)-ABE ecTadA-ecTadA*-Sa KKH nCas9), ABEmax (ecTadA-ecTadA*-Sp nCas9), ABE7.10max (ecTadA-ecTadA*-SpnCas9), ABE8e)ecTadA-ecTadA*-SpnCas9), PE1 (dSpCas9-MMLV-RT), PE2 (dSpCas9-MMLV-RT), PE3 (nSpCas9-MMLV-RT), and BE-PLUS (10× GCN4-Sp nCas9(D10A)/ScFv-rAPOBEC1-UGI (see, e.g., Jiang 2018 Cell Res. 28(8): 855-861)).

In some embodiments, lipid nucleic acid assemblies include an mRNA that encode and/or expresses a uracil DNA glycosylase inhibitor (UGI), e.g., where the lipid nucleic acid assembly also includes an mRNA encoding a cytosine base editor (e.g., where the base editor does not include a DNA glycosylase inhibitor). A DNA glycosylase inhibitor can override natural DNA repair mechanisms that might otherwise repair the intended base editing, decrease generation of indels, and/or allow more efficient deamination of target nucleic acids than base editing in the absence of a UGI. A DNA glycosylase inhibitor can be a uracil DNA glycosylase inhibitor protein (UGI). One exemplary UGI is described in Wang (1991 Gene 99:31-37).

An mRNA can include one or more of a 5′ cap, a 5′ untranslated region (UTR), a 3′ UTRs, and a polyadenine tail. The mRNA can include a modified open reading frame, for example to encode a nuclear localization sequence or to use alternate codons to encode the protein.

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids also include the incorporation of modified uridines such as 5-methoxyuridine, pseudouridine, or N1-methylpseudouridine, or others. The incorporation of pseudouridines into the mRNA nucleic acids may enhance stability and translational capacity, as well as diminishing immunogenicity in vivo. See, e.g., Karikó, K., et al., Molecular Therapy 16 (11): 1833-1840 (2008). Substitutions and modifications to the mRNA may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible).

The term modification also includes, for example, the incorporation of non-nucleotide linkages or modified nucleotides into the mRNA sequences (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, a long poly A tail may be added to an mRNA molecule thus rendering the mRNA more stable. Poly A tails can be added using a variety of art-recognized techniques. For example, long poly A tails can be added to synthetic or in vitro transcribed mRNA using poly A polymerase (Yokoe, et al. Nature Biotechnology. 1996; 14: 1252-1256). A transcription vector can also encode long poly A tails. In addition, poly A tails can be added by transcription directly from PCR products. In some embodiments, the length of the poly A tail is at least about 90, 200, 300, 400 at least 500 nucleotides. In certain embodiments, the length of the poly A tail is adjusted to control the stability of a modified mRNA molecule and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In some embodiments, the stabilized mRNA molecules are sufficiently resistant to in vivo degradation (e.g., by nucleases), such that they may be delivered to the target cell without a transfer vehicle.

In certain embodiments, an mRNA can be modified by the incorporation 3′ and/or 5′ untranslated (UTR) sequences which are not naturally found in the wild-type mRNA. In some embodiments, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

More detailed descriptions of the mRNA modifications can be found in US2017/0210698A1, at pages 57-68, the contents of which are incorporated herein.

In some embodiments, an RNA molecule can be or include a guide RNA (gRNA). In some embodiments, an RNA molecule (e.g., a gRNA) can be or include a CRISPR RNA (crRNA). In various embodiments, crRNA can target a site within a genome based on complementarity. In some embodiments, an RNA molecule (e.g., a gRNA) can be or include a trans-activating CRISPR RNA (tracrRNA) that can associate with an endonuclease. In some embodiments, a gRNA includes both crRNA and tracrRNA. A gRNA that includes both crRNA and tracrRNA can be referred to as a single gRNA (sgRNA). An sgRNA can target a nuclease (e.g., a Cas nuclease) to a desired sequence. Thus, the CRISPR/Cas system can be engineered to create a double-strand break at a desired target in a genome of a cell, and harness the cell's endogenous mechanisms to repair the induced break by HDR, or NHEJ.

In certain embodiments, nucleic acid molecules of the present disclosure can include modified nucleosides or nucleotides. Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-natural and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the polynucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In certain embodiments, a gRNA includes at least one modified residue. In certain embodiments, an mRNA includes at least one modified residue. In certain embodiments, the modified gRNA comprises a modification at one or more of the first five nucleotides at a 5′ end. In certain embodiments, the modified gRNA comprises a modification at one or more of the first five nucleotides at a 3′ end.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g. mRNAs, gRNAs) described herein can contain one or more modified nucleosides or nucleotides, e.g., to introduce stability toward intracellular or serum-based nucleases. In some embodiments, the modified RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

Accordingly, in some embodiments, an RNA or nucleic acid comprises at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” and such terms relate to the nucleic acids described herein, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA or nucleic acid in the target cell, tissue, subject and/or cytoplasm. The stabilized RNA or nucleic acid molecules provided herein demonstrate longer half-lives relative to their naturally occurring, unmodified counterparts (e.g. the wild-type version of the molecule). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the RNA or nucleic acid has undergone a chemical or biological modification to render it more stable. Exemplary modifications to an RNA or nucleic acid include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA or nucleic acids, for example, covalent modifications such as the introduction of modified nucleotides, (e.g., nucleotide analogs, or the inclusion of pendant groups which are not naturally found in such RNA, such as a deoxynucleoside, or nucleic acid molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

In various embodiments, an mRNA nucleotide sequence encoding a polypeptide (e.g., an RNA-guided DNA binding agent, an endonuclease, a base editor, and/or a DNA glycosylase inhibitor (e.g., UGI)) includes a number of nucleotides that is between about 1000 and about 7000. In various embodiments, an mRNA nucleotide sequence encoding a polypeptide includes a number of nucleotides that is, or is greater than, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, about 3000, about 3050, about 3100, about 3150, about 3200, about 3250, about 3300, about 3350, about 3400, about 3450, about 3500, about 3550, about 3600, about 3650, about 3700, about 3750, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, or about 7000.

In various embodiments, an mRNA nucleotide sequence encoding a polypeptide includes a number of nucleotides that is between about 500 and about 7000, about 1000 and about 6000, about 1000 and about 5000, about 1000 and about 4000, about 1000 and about 3000, or about 1000 and about 2000. In various embodiments, an mRNA nucleotide sequence encoding a polypeptide includes a number of nucleotides that is between about 2000 and about 7000, about 2000 and about 6000, about 2000 and about 5000, about 2000 and about 4000, or about 2000 and about 3000. In various embodiments, an mRNA nucleotide sequence encoding a polypeptide includes a number of nucleotides that is between about 3000 and about 7000, about 3000 and about 6000, about 3000 and about 5000, or about 3000 and about 4000. In various embodiments, an mRNA nucleotide sequence encoding a polypeptide includes a number of nucleotides that is between about 4000 and about 7000, about 4000 and about 6000, or about 4000 and about 5000. In various embodiments, an mRNA nucleotide sequence encoding a polypeptide includes a number of nucleotides that is between about 5000 and about 7000 or about 5000 and about 6000. In various embodiments, an mRNA nucleotide sequence encoding a polypeptide includes a number of nucleotides that is between about 500 and about 1000 or about 500 and about 2000. In some embodiments, a gRNA includes a number of nucleotides that is between about 50 and about 200, between about 75 and about 200, between about 50 and about 150, between about 75 and about 150, between about 80 and about 150, between about 50 and about 140, between about 75 and about 140, between about 80 and about 140, between about 75 and about 120, between about 80 and about 130, or between about 80 and about 120. In some embodiments, a gRNA includes a number of nucleotides that is between about 85 and about 115, between about 90 and about 110, or between about 95 and 105. In various embodiments, a gRNA is or includes 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nucleotides.

In various embodiments, an mRNA nucleotide sequence encoding a base editor includes a number of nucleotides that is between about 3000 and about 7000. In various embodiments, an mRNA nucleotide sequence encoding a base editor includes a number of nucleotides that is, or is greater than, about 3000, about 3050, about 3100, about 3150, about 3200, about 3250, about 3300, about 3350, about 3400, about 3450, about 3500, about 3550, about 3600, about 3650, about 3700, about 3750, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, or about 7000.

In various embodiments, an mRNA nucleotide sequence encoding a base editor includes a number of nucleotides that is between about 3000 and about 7000, about 3000 and about 6500, about 3000 and about 6000, about 3000 and about 5500, about 3000 and about 5000, about 3000 and about 4500, or about 3000 and about 4000. In various embodiments, an mRNA nucleotide sequence encoding a base editor includes a number of nucleotides that is between about 4000 and about 7000, 4000 and about 6500, about 4000 and about 6000, 4000 and about 5500, or about 4000 and about 5000.

In various embodiments, an mRNA nucleotide sequence encoding a DNA glycosylase inhibitor includes a number of nucleotides that is between about 500 and about 3000. In various embodiments, an mRNA nucleotide sequence encoding a DNA glycosylase inhibitor includes a number of nucleotides that is, or is greater than, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, or about 3000.

In various embodiments, an mRNA nucleotide sequence encoding a DNA glycosylase inhibitor includes a number of nucleotides that is between about 500 and about 3000, about 500 and about 2500, about 500 and about 2000, about 500 and about 1500, about 500 and about 1000, about 750 and about 3000, about 750 and about 2500, about 750 and about 2000, about 750 and about 1500, about 750 and about 1000, about 1000 and about 3000, about 1000 and about 2500, about 1000 and about 2000, or about 1000 and 1500.

In some embodiments, a gRNA includes a number of nucleotides that is between about 50 and about 200, between about 75 and about 200, between about 50 and about 150, between about 75 and about 150, between about 80 and about 150, between about 50 and about 140, between about 75 and about 140, between about 80 and about 140, between about 75 and about 120, between about 80 and about 130, or between about 80 and about 120. In some embodiments, a gRNA includes a number of nucleotides that is between about 85 and about 115, between about 90 and about 110, or between about 95 and 105. In various embodiments, a gRNA is or includes 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nucleotides.

In various embodiments, a nucleic acid (e.g., a first nucleic acid) includes a number of nucleotides that is between about 1000 and about 7000. In various embodiments, a nucleic acid (e.g., a first nucleic acid) includes a number of nucleotides that is, or is greater than, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, about 3000, about 3050, about 3100, about 3150, about 3200, about 3250, about 3300, about 3350, about 3400, about 3450, about 3500, about 3550, about 3600, about 3650, about 3700, about 3750, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, or about 7000.

In various embodiments, a nucleic acid (e.g., a first nucleic acid) includes a number of nucleotides that is between about 500 and about 7000, about 1000 and about 6000, about 1000 and about 5000, about 1000 and about 4000, about 1000 and about 3000, or about 1000 and about 2000. In various embodiments, a nucleic acid (e.g., a first nucleic acid) includes a number of nucleotides that is between about 2000 and about 7000, about 2000 and about 6000, about 2000 and about 5000, about 2000 and about 4000, or about 2000 and about 3000. In various embodiments, a nucleic acid (e.g., a first nucleic acid) includes a number of nucleotides that is between about 3000 and about 7000, about 3000 and about 6000, about 3000 and about 5000, or about 3000 and about 4000. In various embodiments, a nucleic acid (e.g., a first nucleic acid) includes a number of nucleotides that is between about 4000 and about 7000, about 4000 and about 6000, or about 4000 and about 5000. In various embodiments, a nucleic acid (e.g., a first nucleic acid) includes a number of nucleotides that is between about 5000 and about 7000 or about 5000 and about 6000. In various embodiments, a nucleic acid (e.g., a first nucleic acid) includes a number of nucleotides that is between about 500 and about 1000 or about 500 and about 2000. In some embodiments, a nucleic acid (e.g., a second nucleic acid) includes a number of nucleotides that is between about 50 and about 200, between about 75 and about 200, between about 50 and about 150, between about 75 and about 150, between about 80 and about 150, between about 50 and about 140, between about 75 and about 140, between about 80 and about 140, between about 75 and about 120, between about 80 and about 130, or between about 80 and about 120. In some embodiments, a nucleic acid (e.g., a second nucleic acid) includes a number of nucleotides that is between about 85 and about 115, between about 90 and about 110, or between about 95 and 105. In various embodiments, a nucleic acid (e.g., a second nucleic acid) is or includes 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nucleotides.

In various embodiments, a nucleic acid (e.g., a first nucleic acid) includes a number of nucleotides that is between about 3000 and about 7000. In various embodiments, a nucleic acid (e.g., a first nucleic acid) includes a number of nucleotides that is, or is greater than, about 3000, about 3050, about 3100, about 3150, about 3200, about 3250, about 3300, about 3350, about 3400, about 3450, about 3500, about 3550, about 3600, about 3650, about 3700, about 3750, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, or about 7000.

In various embodiments, a nucleic acid (e.g., a first nucleic acid) includes a number of nucleotides that is between about 3000 and about 7000, about 3000 and about 6500, about 3000 and about 6000, about 3000 and about 5500, about 3000 and about 5000, about 3000 and about 4500, or about 3000 and about 4000. In various embodiments, a nucleic acid (e.g., a first nucleic acid) includes a number of nucleotides that is between about 4000 and about 7000, 4000 and about 6500, about 4000 and about 6000, 4000 and about 5500, or about 4000 and about 5000.

In some embodiments, a nucleic acid (e.g., a second nucleic acid) includes a number of nucleotides that is between about 50 and about 200, between about 75 and about 200, between about 50 and about 150, between about 75 and about 150, between about 80 and about 150, between about 50 and about 140, between about 75 and about 140, between about 80 and about 140, between about 75 and about 120, between about 80 and about 130, or between about 80 and about 120. In some embodiments, a nucleic acid (e.g., a second nucleic acid) includes a number of nucleotides that is between about 85 and about 115, between about 90 and about 110, or between about 95 and 105. In various embodiments, a nucleic acid (e.g., a second nucleic acid) is or includes 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nucleotides.

In various embodiments, a nucleic acid (e.g., a third nucleic acid) includes a number of nucleotides that is between about 500 and about 3000. In various embodiments, a nucleic acid (e.g., a third nucleic acid) includes a number of nucleotides that is, or is greater than, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, or about 3000.

In various embodiments, a nucleic acid (e.g., a third nucleic acid) includes a number of nucleotides that is between about 500 and about 3000, about 500 and about 2500, about 500 and about 2000, about 500 and about 1500, about 500 and about 1000, about 750 and about 3000, about 750 and about 2500, about 750 and about 2000, about 750 and about 1500, about 750 and about 1000, about 1000 and about 3000, about 1000 and about 2500, about 1000 and about 2000, or about 1000 and 1500.

In various embodiments, a lipid nucleic acid assembly composition includes a gRNA and an mRNA (e.g., one mRNA or two mRNAs) where the difference in length between the gRNA and the mRNA (e.g., between the gRNA and a first mRNA, between the gRNA and a second mRNA, or between the gRNA and each of a first mRNA and a second mRNA, independently) is at least or about 500 nucleotides to about 7000 nucleotides. In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is at least or about 500, at least or about 550, at least or about 600, at least or about 650, at least or about 700, at least or about 750, at least or about 800, at least or about 850, at least or about 900, at least or about 950, at least or about 1000, at least or about 1050, at least or about 1100, at least or about 1150, at least or about 1200, at least or about 1250, at least or about 1300, at least or about 1350, at least or about 1400, at least or about 1450, at least or about 1500, at least or about 1550, at least or about 1600, at least or about 1650, at least or about 1700, at least or about 1750, at least or about 1800, at least or about 1850, at least or about 1900, at least or about 1950, at least or about 2000, at least or about 2050, at least or about 2100, at least or about 2150, at least or about 2200, at least or about 2250, at least or about 2300, at least or about 2350, at least or about 2400, at least or about 2450, at least or about 2500, at least or about 2550, at least or about 2600, at least or about 2650, at least or about 2700, at least or about 2750, at least or about 2800, at least or about 2850, at least or about 2900, at least or about 2950, at least or about 3000, at least or about 3050, at least or about 3100, at least or about 3150, at least or about 3200, at least or about 3250, at least or about 3300, at least or about 3350, at least or about 3400, at least or about 3450, at least or about 3500, at least or about 3550, at least or about 3600, at least or about 3650, at least or about 3700, at least or about 3750, at least or about 3800, at least or about 3850, at least or about 3900, at least or about 3950, at least or about 4000, at least or about 4050, at least or about 4100, at least or about 4150, at least or about 4200, at least or about 4250, at least or about 4300, at least or about 4350, at least or about 4400, at least or about 4450, at least or about 4500, at least or about 4550, at least or about 4600, at least or about 4650, at least or about 4700, at least or about 4750, at least or about 4800, at least or about 4850, at least or about 4900, at least or about 4950, at least or about 5000, at least or about 5050, at least or about 5100, at least or about 5150, at least or about 5200, at least or about 5250, at least or about 5300, at least or about 5350, at least or about 5400, at least or about 5450, at least or about 5500, at least or about 5550, at least or about 5600, at least or about 5650, at least or about 5700, at least or about 5750, at least or about 5800, at least or about 5850, at least or about 5900, at least or about 5950, at least or about 6000, at least or about 6050, at least or about 6100, at least or about 6150, at least or about 6200, at least or about 6250, at least or about 6300, at least or about 6350, at least or about 6400, at least or about 6450, at least or about 6500, at least or about 6550, at least or about 6600, at least or about 6650, at least or about 6700, at least or about 6750, at least or about 6800, at least or about 6850, at least or about 6900, at least or about 6950, or at least or about 7000.

In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is a number of nucleotides that is, or is at least, between about 500 and about 7000, about 500 and about 6000, about 500 and about 5000, about 500 and about 4000, about 500 and about 3000, about 500 and about 2000, or about 500 and about 1000. In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is a number of nucleotides that is, or is at least, between about 500 and about 7000, about 1000 and about 7000, about 1000 and about 6000, about 1000 and about 5000, about 1000 and about 4000, about 1000 and about 3000, about 1000 and about 2000, or about 500 and about 1000. In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is a number of nucleotides that is, or is at least, between about 2000 and about 7000, about 2000 and about 6000, about 2000 and about 5000, about 2000 and about 4000, or about 2000 and about 3000. In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is a number of nucleotides that is, or is at least, between about 3000 and about 7000, about 3000 and about 6000, about 3000 and about 5000, or about 3000 and about 4000. In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is a number of nucleotides that is, or is at least, between about 4000 and about 7000, about 4000 and about 6000, or about 4000 and about 5000. In various embodiments, a lipid nucleic acid assembly composition includes a gRNA and an mRNA where the difference in length between the gRNA and the mRNA (e.g., between the gRNA and a first mRNA, between the gRNA and a second mRNA, or between the gRNA and each of a first mRNA and a second mRNA, independently) is a number of nucleotides that is, or is at least, between about 5000 and about 7000 or about 5000 and about 6000.

In various embodiments, a lipid nucleic acid assembly composition includes a first mRNA and a second mRNA (e.g., a first mRNA encoding a cytosine base editor and a second mRNA encoding a DNA glycosylase inhibitor) where the difference in length between the first mRNA and the second mRNA is at least or about 500 nucleotides to about 7000 nucleotides. In various embodiments, a lipid nucleic acid assembly composition includes a first mRNA and a second mRNA where the difference in length between the first mRNA and the second mRNA is at least or about 500, at least or about 550, at least or about 600, at least or about 650, at least or about 700, at least or about 750, at least or about 800, at least or about 850, at least or about 900, at least or about 950, at least or about 1000, at least or about 1050, at least or about 1100, at least or about 1150, at least or about 1200, at least or about 1250, at least or about 1300, at least or about 1350, at least or about 1400, at least or about 1450, at least or about 1500, at least or about 1550, at least or about 1600, at least or about 1650, at least or about 1700, at least or about 1750, at least or about 1800, at least or about 1850, at least or about 1900, at least or about 1950, at least or about 2000, at least or about 2050, at least or about 2100, at least or about 2150, at least or about 2200, at least or about 2250, at least or about 2300, at least or about 2350, at least or about 2400, at least or about 2450, at least or about 2500, at least or about 2550, at least or about 2600, at least or about 2650, at least or about 2700, at least or about 2750, at least or about 2800, at least or about 2850, at least or about 2900, at least or about 2950, at least or about 3000, at least or about 3050, at least or about 3100, at least or about 3150, at least or about 3200, at least or about 3250, at least or about 3300, at least or about 3350, at least or about 3400, at least or about 3450, at least or about 3500, at least or about 3550, at least or about 3600, at least or about 3650, at least or about 3700, at least or about 3750, at least or about 3800, at least or about 3850, at least or about 3900, at least or about 3950, at least or about 4000, at least or about 4050, at least or about 4100, at least or about 4150, at least or about 4200, at least or about 4250, at least or about 4300, at least or about 4350, at least or about 4400, at least or about 4450, at least or about 4500, at least or about 4550, at least or about 4600, at least or about 4650, at least or about 4700, at least or about 4750, at least or about 4800, at least or about 4850, at least or about 4900, at least or about 4950, at least or about 5000, at least or about 5050, at least or about 5100, at least or about 5150, at least or about 5200, at least or about 5250, at least or about 5300, at least or about 5350, at least or about 5400, at least or about 5450, at least or about 5500, at least or about 5550, at least or about 5600, at least or about 5650, at least or about 5700, at least or about 5750, at least or about 5800, at least or about 5850, at least or about 5900, at least or about 5950, at least or about 6000, at least or about 6050, at least or about 6100, at least or about 6150, at least or about 6200, at least or about 6250, at least or about 6300, at least or about 6350, at least or about 6400, at least or about 6450, at least or about 6500, at least or about 6550, at least or about 6600, at least or about 6650, at least or about 6700, at least or about 6750, at least or about 6800, at least or about 6850, at least or about 6900, at least or about 6950, or at least or about 7000.

In various embodiments, a lipid nucleic acid assembly composition includes a first mRNA and a second mRNA where the difference in length between the first mRNA and the second mRNA is a number of nucleotides that is, or is at least, between about 500 and about 7000, about 500 and about 6000, about 500 and about 5000, about 500 and about 4000, about 500 and about 3000, about 500 and about 2000, or about 500 and about 1000. In various embodiments, a lipid nucleic acid assembly composition includes a first mRNA and a second mRNA where the difference in length between the first mRNA and the second mRNA is a number of nucleotides that is, or is at least, between about 1000 and about 7000, about 1000 and about 6000, about 1000 and about 5000, about 1000 and about 4000, about 1000 and about 3000, or about 1000 and about 2000. In various embodiments, a lipid nucleic acid assembly composition includes a first mRNA and a second mRNA where the difference in length between the first mRNA and the second mRNA is a number of nucleotides that is, or is at least, between about 2000 and about 7000, about 2000 and about 6000, about 2000 and about 5000, about 2000 and about 4000, or about 2000 and about 3000. In various embodiments, a lipid nucleic acid assembly composition includes a first mRNA and a second mRNA where the difference in length between the first mRNA and the second mRNA is a number of nucleotides that is, or is at least, between about 3000 and about 7000, about 3000 and about 6000, about 3000 and about 5000, or about 3000 and about 4000. In various embodiments, a lipid nucleic acid assembly composition includes a first mRNA and a second mRNA where the difference in length between the first mRNA and the second mRNA is a number of nucleotides that is, or is at least, between about 4000 and about 7000, about 4000 and about 6000, or about 4000 and about 5000. In various embodiments, a lipid nucleic acid assembly composition includes a first mRNA and a second mRNA where the difference in length between the first mRNA and the second mRNA is a number of nucleotides that is, or is at least, between about 5000 and about 7000 or about 5000 and about 6000.

In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is at least or about 500 nucleotides to about 7000 nucleotides. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is at least or about 500, at least or about 550, at least or about 600, at least or about 650, at least or about 700, at least or about 750, at least or about 800, at least or about 850, at least or about 900, at least or about 950, at least or about 1000, at least or about 1050, at least or about 1100, at least or about 1150, at least or about 1200, at least or about 1250, at least or about 1300, at least or about 1350, at least or about 1400, at least or about 1450, at least or about 1500, at least or about 1550, at least or about 1600, at least or about 1650, at least or about 1700, at least or about 1750, at least or about 1800, at least or about 1850, at least or about 1900, at least or about 1950, at least or about 2000, at least or about 2050, at least or about 2100, at least or about 2150, at least or about 2200, at least or about 2250, at least or about 2300, at least or about 2350, at least or about 2400, at least or about 2450, at least or about 2500, at least or about 2550, at least or about 2600, at least or about 2650, at least or about 2700, at least or about 2750, at least or about 2800, at least or about 2850, at least or about 2900, at least or about 2950, at least or about 3000, at least or about 3050, at least or about 3100, at least or about 3150, at least or about 3200, at least or about 3250, at least or about 3300, at least or about 3350, at least or about 3400, at least or about 3450, at least or about 3500, at least or about 3550, at least or about 3600, at least or about 3650, at least or about 3700, at least or about 3750, at least or about 3800, at least or about 3850, at least or about 3900, at least or about 3950, at least or about 4000, at least or about 4050, at least or about 4100, at least or about 4150, at least or about 4200, at least or about 4250, at least or about 4300, at least or about 4350, at least or about 4400, at least or about 4450, at least or about 4500, at least or about 4550, at least or about 4600, at least or about 4650, at least or about 4700, at least or about 4750, at least or about 4800, at least or about 4850, at least or about 4900, at least or about 4950, at least or about 5000, at least or about 5050, at least or about 5100, at least or about 5150, at least or about 5200, at least or about 5250, at least or about 5300, at least or about 5350, at least or about 5400, at least or about 5450, at least or about 5500, at least or about 5550, at least or about 5600, at least or about 5650, at least or about 5700, at least or about 5750, at least or about 5800, at least or about 5850, at least or about 5900, at least or about 5950, at least or about 6000, at least or about 6050, at least or about 6100, at least or about 6150, at least or about 6200, at least or about 6250, at least or about 6300, at least or about 6350, at least or about 6400, at least or about 6450, at least or about 6500, at least or about 6550, at least or about 6600, at least or about 6650, at least or about 6700, at least or about 6750, at least or about 6800, at least or about 6850, at least or about 6900, at least or about 6950, or at least or about 7000.

In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 500 and about 7000, about 500 and about 6000, about 500 and about 5000, about 500 and about 4000, about 500 and about 3000, about 500 and about 2000, or about 500 and about 1000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 500 and about 7000, about 1000 and about 7000, about 1000 and about 6000, about 1000 and about 5000, about 1000 and about 4000, about 1000 and about 3000, about 1000 and about 2000, or about 500 and about 1000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 2000 and about 7000, about 2000 and about 6000, about 2000 and about 5000, about 2000 and about 4000, or about 2000 and about 3000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 3000 and about 7000, about 3000 and about 6000, about 3000 and about 5000, or about 3000 and about 4000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 4000 and about 7000, about 4000 and about 6000, or about 4000 and about 5000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 5000 and about 7000 or about 5000 and about 6000.

In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is at least or about 500 nucleotides to about 7000 nucleotides. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is at least or about 500, at least or about 550, at least or about 600, at least or about 650, at least or about 700, at least or about 750, at least or about 800, at least or about 850, at least or about 900, at least or about 950, at least or about 1000, at least or about 1050, at least or about 1100, at least or about 1150, at least or about 1200, at least or about 1250, at least or about 1300, at least or about 1350, at least or about 1400, at least or about 1450, at least or about 1500, at least or about 1550, at least or about 1600, at least or about 1650, at least or about 1700, at least or about 1750, at least or about 1800, at least or about 1850, at least or about 1900, at least or about 1950, at least or about 2000, at least or about 2050, at least or about 2100, at least or about 2150, at least or about 2200, at least or about 2250, at least or about 2300, at least or about 2350, at least or about 2400, at least or about 2450, at least or about 2500, at least or about 2550, at least or about 2600, at least or about 2650, at least or about 2700, at least or about 2750, at least or about 2800, at least or about 2850, at least or about 2900, at least or about 2950, at least or about 3000, at least or about 3050, at least or about 3100, at least or about 3150, at least or about 3200, at least or about 3250, at least or about 3300, at least or about 3350, at least or about 3400, at least or about 3450, at least or about 3500, at least or about 3550, at least or about 3600, at least or about 3650, at least or about 3700, at least or about 3750, at least or about 3800, at least or about 3850, at least or about 3900, at least or about 3950, at least or about 4000, at least or about 4050, at least or about 4100, at least or about 4150, at least or about 4200, at least or about 4250, at least or about 4300, at least or about 4350, at least or about 4400, at least or about 4450, at least or about 4500, at least or about 4550, at least or about 4600, at least or about 4650, at least or about 4700, at least or about 4750, at least or about 4800, at least or about 4850, at least or about 4900, at least or about 4950, at least or about 5000, at least or about 5050, at least or about 5100, at least or about 5150, at least or about 5200, at least or about 5250, at least or about 5300, at least or about 5350, at least or about 5400, at least or about 5450, at least or about 5500, at least or about 5550, at least or about 5600, at least or about 5650, at least or about 5700, at least or about 5750, at least or about 5800, at least or about 5850, at least or about 5900, at least or about 5950, at least or about 6000, at least or about 6050, at least or about 6100, at least or about 6150, at least or about 6200, at least or about 6250, at least or about 6300, at least or about 6350, at least or about 6400, at least or about 6450, at least or about 6500, at least or about 6550, at least or about 6600, at least or about 6650, at least or about 6700, at least or about 6750, at least or about 6800, at least or about 6850, at least or about 6900, at least or about 6950, or at least or about 7000.

In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 500 and about 7000, about 500 and about 6000, about 500 and about 5000, about 500 and about 4000, about 500 and about 3000, about 500 and about 2000, or about 500 and about 1000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 1000 and about 7000, about 1000 and about 6000, about 1000 and about 5000, about 1000 and about 4000, about 1000 and about 3000, or about 1000 and about 2000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 2000 and about 7000, about 2000 and about 6000, about 2000 and about 5000, about 2000 and about 4000, or about 2000 and about 3000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 3000 and about 7000, about 3000 and about 6000, about 3000 and about 5000, or about 3000 and about 4000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 4000 and about 7000, about 4000 and about 6000, or about 4000 and about 5000. In various embodiments, a lipid nucleic acid assembly composition includes a first nucleic acid, a second nucleic acid, and optionally a third nucleic acid, where the difference in length between the first nucleic acid and the second nucleic acid, between the first nucleic acid and the third nucleic acid (if present), or between the second nucleic acid and the third nucleic acid (if present) is a number of nucleotides that is, or is at least, between about 5000 and about 7000 or about 5000 and about 6000.

In certain exemplary embodiments, a lipid nucleic acid assembly composition can include a gRNA (e.g., a gRNA of about 80 to about 120 nucleotides in length), a nucleic acid encoding a DNA glycosylase inhibitor such as UGI (e.g., an mRNA of about 1000 nucleotides in length), and a nucleic acid encoding a cytosine base editor (e.g., an mRNA of about 4500 nucleotides in length).

In various embodiments, a lipid nucleic acid assembly composition can include a total nucleic acid concentration and/or a total RNA concentration that is at least, equal to, or less than about 3 mg/mL, 2.5 mg/mL, 2.0 mg/mL, 1.5 mg/mL, 1.0 mg/mL, 0.5 mg/mL, 0.4 mg/mL, or 0.3 mg/mL. In various embodiments, a lipid nucleic acid assembly composition can include a total nucleic acid concentration and/or a total RNA concentration that is between 0.3 and 2.5 mg/mL. In various embodiments, a lipid nucleic acid assembly composition can include a total nucleic acid concentration and/or a total RNA concentration that is between 0.75 mg/mL and 1.8 mg/mL or between about 0.5 mg/mL and about 2 mg/mL. In various embodiments, a lipid nucleic acid assembly composition can include a total nucleic acid concentration and/or a total RNA concentration that is about 1.5 mg/mL

In various embodiments, a lipid nucleic acid assembly composition can include a concentration of an mRNA that is at least, equal to, or less than about 3 mg/mL, 2.5 mg/mL, 2.0 mg/mL, 1.5 mg/mL, 1.0 mg/mL, 0.5 mg/mL, 0.4 mg/mL, or 0.3 mg/mL. In various embodiments, a lipid nucleic acid assembly composition can include a concentration of an mRNA that is between 0.3 and 2.5 mg/mL or between about 0.5 mg/mL and about 2 mg/mL. In various embodiments, a lipid nucleic acid assembly composition can include a concentration of an mRNA that is between 0.75 mg/mL and 1.8 mg/mL. In various embodiments, a lipid nucleic acid assembly composition can include a concentration of an mRNA that is about 1.5 mg/mL.

In various embodiments, a lipid nucleic acid assembly composition can include a concentration of an sgRNA that at least, equal to, or less than about 3 mg/mL, 2.5 mg/mL, 2.0 mg/mL, 1.5 mg/mL, 1.0 mg/mL, 0.5 mg/mL, 0.4 mg/mL, or 0.3 mg/mL. In various embodiments, a lipid nucleic acid assembly composition can include a concentration of an sgRNA that is between 0.3 and 2.5 mg/mL or between about 0.5 mg/mL and about 2 mg/mL. In various embodiments, a lipid nucleic acid assembly composition can include a concentration of an sgRNA that is between 0.75 mg/mL and 1.8 mg/mL. In various embodiments, a lipid nucleic acid assembly composition can include a concentration of an sgRNA that is about 1.5 mg/mL.

In various embodiments in which a lipid nucleic acid assembly composition includes an mRNA and an sgRNA, the ratio of mRNA:sgRNA can be between about 1:1 and about 5:1, e.g., where the ratio of mRNA:sgRNA can be about 3:2, 2:1, about 3:1, or about 4:1. In certain particular embodiments, the ratio of mRNA:sgRNA can be about 2:1. In certain particular embodiments, the ratio of mRNA:sgRNA can be about 3:2.

The present disclosure includes that any reagent or material disclosed herein or utilized in methods provided herein that contacts or may contact lipid nucleic acid assembly compositions and/or nucleic acids can be free or substantially free of RNase.

Extraction of Nucleic Acids from Lipid Nucleic Acid Assembly Compositions

In various embodiments, the present disclosure includes extraction of nucleic acid from a lipid nucleic acid assembly composition. Extraction of nucleic acid produces a nucleic acid extract, such that the nucleic acid extract includes a portion or subset of the material present in the lipid nucleic acid assembly composition. Extraction of nucleic acid from a lipid nucleic acid assembly composition includes techniques that isolate or purify one or more, or all, nucleic acids present in a lipid nucleic acid assembly composition. The present disclosure includes the unexpected discovery, that, among other things, nucleic acid extraction according to a technique that includes ethanol precipitation, isopropyl alcohol precipitation, butanol precipitation, or acetonitrile precipitation can be particularly advantageous in certain contexts, e.g., for the purification of nucleic acids such as RNA from lipid nucleic acid assembly compositions including LNPs, e.g., where the nucleic acid extract is subsequently subjected to chromatography. While the present disclosure includes exemplification of ethanol precipitation as one preferred embodiment for use in nucleic acid extraction, the present disclosure includes, as will be understood by those of skill in the art, that similar results and advantages are achieved using isopropyl alcohol precipitation, butanol precipitation, or acetonitrile precipitation.

A number of nucleic acid extraction techniques are known in the art. For example, techniques for extraction of nucleic acid from lipid nucleic acid assembly compositions in which the nucleic acid is present in an encapsulating structure (e.g., cells) can include mechanical and/or chemical steps to release nucleic acids from encapsulating structures, e.g., by grinding, freeze-thawing, bead-beating, enzymatic treatment (e.g., with hydrolases), or exposure to detergents. Subsequent to such steps for mechanical or chemical release of nucleic acids from the encapsulating structure, purification of nucleic acid from the processed lipid nucleic acid assembly composition can include separate washing, filtration, and/or precipitation with any of a variety of solutions, buffers, and/or detergents, such as phenol, chloroform, or cetyltrimethylammonium bromide, polyethylene glycol, magnetic beads, ion-exchange resins, or gels. Various such approaches can entail significant commitment of time and resources for preparation of nucleic acid extracts. Moreover, as noted above, various such approaches can be incompatible with downstream applications. For example, various prior techniques for extraction of nucleic acids (e.g., RNA, e.g., from LNPs) included organic solvents, buffers, and/or detergents that rendered nucleic acid extracts incompatible with chromatography (e.g., SEC or ion pair chromatography, e.g., ion pair reverse phase HPLC) due to one or both of product turbidity, low nucleic acid recovery, inconsistent nucleic acid recovery, and/or inconsistent suitability for chromatography (e.g., in that chromatography results were not reproducible, demonstrated low precision, and/or demonstrated low accuracy). Moreover, various extraction and/or chromatography techniques, when applied to chromatographic purification and/or separation of nucleic acids present in lipid nucleic acid assembly composition, can result in and/or promote carryover. Carryover is characterized by the detection of analyte from a prior sample during analysis of a subsequent sample using the same apparatus. The present disclosure thus includes, among other things, the appreciation that there was a problem of identifying protocols for extracting nucleic acids (e.g., RNA, e.g., from LNPs) in a manner that produced a nucleic acid extract compatible with, useful for, and/or improved for use in chromatography (e.g., SEC or ion pair chromatography, e.g., ion pair reverse phase HPLC).

The present disclosure includes methods of nucleic acid extraction that are characterized by unexpected advantageous properties for production of nucleic acid extracts, including without limitation production of nucleic acid extracts that are compatible with chromatography (e.g., SEC or ion pair chromatography, e.g., ion pair reverse phase HPLC). The present disclosure further includes methods characterized by unexpected reduction in carryover. The present disclosure includes methods of nucleic acid extraction in which nucleic acid is isolated by ethanol precipitation, isopropyl alcohol precipitation, butanol precipitation, or acetonitrile precipitation. In one exemplary nucleic acid extraction of the present disclosure, a lipid nucleic acid assembly composition including nucleic acids (e.g., a lipid nucleic acid assembly composition in which nucleic acids are encapsulated in LNPs) is mixed with ethanol to produce an ethanol mixture, the ethanol mixture is centrifuged to produce a nucleic acid pellet, and the nucleic acid pellet is separated from the ethanol to produce an isolated nucleic acid pellet. In one exemplary nucleic acid extraction of the present disclosure, a lipid nucleic acid assembly composition including nucleic acids (e.g., a lipid nucleic acid assembly composition in which nucleic acids are encapsulated in LNPs) is mixed with ethanol to produce an ethanol mixture, the ethanol mixture is centrifuged to produce a nucleic acid pellet, the nucleic acid pellet is separated from the ethanol to produce an isolated nucleic acid pellet, and the isolated nucleic acid pellet is resuspended in an aqueous solution, thereby producing a nucleic acid extract. As is disclosed elsewhere herein, in various embodiments the nucleic acids can be or include two or more RNA molecules, e.g., an mRNA and an sgRNA, one or both of which can be encapsulated in LNPs in the lipid nucleic acid assembly composition. As is disclosed elsewhere herein, in various embodiments the nucleic acids can be or include three or more RNA molecules, e.g., a first mRNA, a second mRNA, and an sgRNA, one or more, or all, of which can be encapsulated in LNPs in the lipid nucleic acid assembly composition.

In various embodiments, ethanol mixed with a lipid nucleic acid assembly composition in a nucleic acid extraction of the present disclosure is 200 proof. In various embodiments, ethanol mixed with a lipid nucleic acid assembly composition in a nucleic acid extraction of the present disclosure can be free or substantially free of RNAse. In various embodiments, ethanol mixed with a sample in a nucleic acid extraction of the present disclosure can include RNAse-free water and/or nuclease-free water. In various embodiments, a lipid nucleic acid assembly composition can optionally be mechanically treated to improve mixing with ethanol, e.g., by shaking, inversion, flicking, or vortexing, e.g., for 1-3 seconds.

In various embodiments, centrifugation used in a nucleic acid extraction of the present disclosure can be performed using at least or about 10000 RCF (e.g., at least or about 10000, at least or about 15000, at least or about 20000, at least or about 25000, or at least or about 30000 RCF). In various embodiments, centrifugation used in a nucleic acid extraction of the present disclosure can be performed for at least or about 10 minutes (e.g., at least or about 10 minutes, at least or about 15 minutes, at least or about 20 minutes, at least or about 25 minutes, or at least or about 30 minutes).

In various embodiments, separation of a nucleic acid from ethanol can be performed by decanting. In various embodiments, separation of a nucleic acid from ethanol can be performed by evaporative drying. In various embodiments, separation of a nucleic acid from ethanol can be performed by vacufuge. In various embodiments, separation of a nucleic acid from ethanol can be performed by pipetting away alcohol. In various embodiments, separation of a nucleic acid from ethanol can be performed by a combination of any of decanting, evaporative drying, vacufuge, and/or pipetting. In various embodiments, separation of a nucleic acid from ethanol can be performed by a person of skill in the art such that no residual ethanol is visible in the vessel holding the nucleic acid pellet. In various embodiments, extraction of nucleic acid and/or separation of a nucleic acid from ethanol can be performed by a person of skill in the art such that no, or essentially no, ethanol remains associated with the nucleic acid pellet.

In various embodiments, steps of mixing ethanol with a lipid nucleic acid assembly composition to produce an ethanol mixture, centrifuging the ethanol mixture to produce a nucleic acid pellet, and separating the nucleic acid pellet from the ethanol to produce an isolated nucleic acid pellet can be repeated. In various embodiments, methods of nucleic acid extraction disclosed herein can include two, three, four, or five repetitions of steps of mixing ethanol with a lipid nucleic acid assembly composition to produce an ethanol mixture, centrifuging the ethanol mixture to produce a nucleic acid pellet, and separating the nucleic acid pellet from the ethanol to produce an isolated nucleic acid pellet. In various embodiments, such repetitions can be consecutive and/or essentially consecutive in time and/or in steps of a protocol.

In various embodiments, an isolated nucleic acid pellet can be resuspended in water. In various embodiments, an isolated nucleic acid pellet can be resuspended in RNAse-free water. In various embodiments, an isolated nucleic acid pellet can be resuspended in nuclease-free water. In various embodiments, a resuspension of the present disclosure is diluted as compared to the lipid nucleic acid assembly composition with respect to nucleic acid concentration (e.g., at least or about 2-fold, at least or about 3-fold, at least or about 4-fold, at least or about 5-fold, or at least or about 10-fold diluted as compared to the lipid nucleic acid assembly composition with respect to nucleic acid concentration). Those of skill in the art will appreciate that a nucleic acid extract can be diluted to a concentration appropriate to an intended use, e.g., to a concentration appropriate for an intended assay or analysis. Those of skill in the art will further appreciate that a nucleic acid extract can be diluted to a concentration that is within the measured or measureable range of an intended assay or analysis, which can be in various embodiments established by user-elected references or controls (e.g., a chosen calibration curve). In various embodiments, a nucleic acid extract can be diluted to a concentration (e.g., a total nucleic acid concentration) that is at or about the midpoint of the concentration (e.g., the total nucleic acid concentration) of a calibration curve.

In various embodiments, a nucleic acid extract can be heated prior to chromatographic purification and/or separation, where the heating causes denaturation. In various embodiments, heating of a nucleic acid extract prior to separation can include heating to a temperature that is between about 65° C. and about 90° C. (e.g., to a temperature of about 65° C., about 70° C., about 75° C., about 80° C., about 85° C., or about 90° C.). In various embodiments, heating of a nucleic acid extract prior to separation can include heating to a temperature that is between about 65° C. and 85° C., between about 65° C. and 80° C., between about 65° C. and 75° C., between about 65° C. and 70° C., between about 70° C. and 90° C., between about 70° C. and 85° C., between about 70° C. and 80° C., or between about 70° C. and 75° C. In various embodiments, heating of a nucleic acid extract prior to separation can include heating to an indicated temperature for a period of at least 2 minutes, e.g., for a period that is between about 2 minutes and about 15 minutes (e.g., about 2 minutes, about 3 minutes, about 4 minutes, about 5 minutes, about 10 minutes, or about 15 minutes). In various embodiments, heating of a nucleic acid extract prior to separation can include heating to an indicated temperature for a period that is between about 5 minutes and 15 minutes.

In various embodiments, a heating step can be followed by a cooling step in which the sample is cooled, e.g., to a temperature between 1° C. and 10° C. (e.g., to about 1° C., about 2° C., about 3° C., about 4° C., about 5° C., about 6° C., about 7° C., about 8° C., about 9° C., or about 10° C.). In various embodiments, cooling of a nucleic acid extract prior to separation can include cooling to a temperature that is between about 1° C. and 5° C., between about 3° C. and 10° C., or between about 3° C. and 5° C. In various embodiments, cooling of a nucleic acid extract prior to separation can include cooling to an indicated temperature for a period of at least 2 minutes, e.g., for a period that is between about 2 minutes and about 15 minutes (e.g., about 2 minutes, about 3 minutes, about 4 minutes, about 5 minutes, about 10 minutes, or about 15 minutes). In various embodiments, cooling of a nucleic acid extract prior to separation can include cooling to an indicated temperature for a period that is between about 5 minutes and 15 minutes. In various embodiments, a nucleic acid extract can be heated and/or cooled prior to transfer to HPLC vials for separation. In various embodiments, nucleic acid extraction according to the present disclosure can include extraction of RNA molecules (e.g., mRNA and sgRNA molecules) from a lipid nucleic acid assembly composition in which the RNA molecules are encapsulated in LNPs, where the nucleic acid is isolated by ethanol precipitation, isopropyl alcohol precipitation, butanol precipitation, or acetonitrile precipitation as disclosed herein. In various embodiments, nucleic acid extraction according to the present disclosure is characterized by unexpected advantageous properties for production of nucleic acid extracts that include, without limitation, that nucleic acid extracts that are compatible with chromatography (e.g., SEC or ion pair chromatography, e.g., ion pair reverse phase HPLC). Without wishing to be bound by any particular scientific theory, at least one reason nucleic acid extracts produced in accordance with the present disclosure are compatible with chromatography (e.g., SEC or ion pair chromatography, e.g., ion pair reverse phase HPLC) can be that the nucleic acid extracts are unexpectedly free or substantially free of lipids, organic solvents, buffers, and/or detergents. Without wishing to be bound by any particular scientific theory, properties of nucleic acid extracts produced in accordance with the present disclosure that contribute to compatibility with chromatography (e.g., SEC or ion pair chromatography, e.g., ion pair reverse phase HPLC) can include low turbidity, high nucleic acid recovery, consistent nucleic acid recovery, and observed downstream properties in chromatography (e.g., SEC or ion pair chromatography, e.g., ion pair reverse phase HPLC) that can include but need not be limited to reproducibility of lipid nucleic acid assembly composition chromatography results, accuracy of lipid nucleic acid assembly composition chromatography results, and/or precision of lipid nucleic acid assembly composition chromatography results.

### Separation of Nucleic Acid Extracts

The present disclosure includes that molecules (e.g., nucleic acid molecules) present in a lipid nucleic acid assembly composition (e.g., present in a nucleic acid extracted derived from a lipid nucleic acid assembly composition) can be purified and/or separated by a variety of methods and/or in accordance with a variety of molecular characteristics, examples of which include chromatography, capillary zone electrophoresis, or capillary isoelectric focusing. Examples of chromatography include gas chromatography, liquid chromatography, high performance liquid chromatography (HPLC), size exclusion chromatography (SEC), affinity chromatography, anion exchange chromatography, cation exchange chromatography, gel filtration chromatography, hydrophobic interaction chromatography, ion exchange chromatography, reverse phase chromatography, paper chromatography, and thin-layer chromatography.

In certain particular embodiments, methods of the present disclosure include purification and/or separation of nucleic acids by size exclusion chromatography. In various embodiments of size exclusion chromatography, an aqueous solution or organic solvent is applied to a substrate (e.g., a substrate present in a chromatography column). The solution can be referred to as the mobile phase and the substrate can be referred to as the stationary phase. Size exclusion chromatography includes chromatography techniques in which molecules are purified and/or separated based on size and/or hydrodynamic volume, where differences cause molecules to elute (filter) through a stationary phase (e.g., a column) at different rates. The present disclosure includes the unexpected observation that use of size exclusion chromatography as provided herein is associated with decreased carryover as compared to other forms of chromatography, e.g., affinity chromatography or use of certain columns other than those of methods and compositions disclosed herein.

In certain particular embodiments, methods of the present disclosure include purification and/or separation of nucleic acids by HPLC. HPLC includes a form of column chromatography in which a pump moves the mobile phase(s) through the column. HPLC can be utilized in combination with a variety of chromatography substrates and various modes of chromatography can therefore be performed using HPLC. For example, SEC can be performed using HPLC.

In various embodiments, methods of the present disclosure include SEC using a substrate and/or column characterized by one or more particular characteristics provided herein. In various embodiments, SEC of the present disclosure includes contacting a nucleic acid extract with an SEC substrate and/or column characterized by a pore size of about 5 nm to about 50 nm, optionally wherein the pore size is about 12.5 nm to about 25 nm. In various embodiments, SEC of the present disclosure includes contacting a nucleic acid extract with an SEC substrate and/or column characterized by an internal diameter of 3 mm to 6 mm, optionally having an internal diameter of about 4 mm to about 5 mm, optionally having an internal diameter of about 4.6 mm. In various embodiments, SEC of the present disclosure includes contacting a nucleic acid extract with an SEC substrate and/or column characterized by a length of about 100 mm to about 400 mm (e.g., about 100 mm, about 150 mm, about 200 mm, about 250 mm, about 300 mm, about 350 mm, or about 400 mm), optionally having a length of about 300 mm length. In various embodiments, SEC of the present disclosure includes contacting a nucleic acid extract with an SEC substrate and/or column characterized by a length of about 200 mm to about 400 mm, about 250 mm to about 350 mm, or about 275 mm to about 325 mm. In various embodiments, SEC of the present disclosure includes contacting a nucleic acid extract with an SEC substrate and/or column characterized by a particle size of about 3 μm to about 5 μm, optionally a particle size of about 4 μm. In various embodiments, SEC of the present disclosure includes contacting a nucleic acid extract with an SEC substrate and/or column characterized by compatibility with an aqueous mobile phase. In various embodiments, SEC of the present disclosure includes contacting a nucleic acid extract with an SEC substrate and/or column characterized by compatibility with a salt-based mobile phase In various embodiments, SEC of the present disclosure includes contacting a nucleic acid extract with an SEC substrate and/or column characterized by compatibility with a mobile phase having a pH of about 7 to about 8, optionally a mobile phase having a pH of about 7.3 to about 7.7, optionally a mobile phase having a pH of about 7.5. In various embodiments, SEC of the present disclosure includes contacting a nucleic acid extract with an SEC substrate and/or column characterized by compatibility with a mobile phase including about 25 mM to about 200 mM NaCl, optionally wherein the SEC is compatible with a mobile phase including about 150 mM NaCl. In various embodiments, SEC of the present disclosure includes contacting a nucleic acid extract with an SEC substrate and/or column characterized by compatibility with an SEC mobile phase having a pH between about 7 and about 8, optionally wherein the mobile phase has a pH of about 7.3 to about 7.7, optionally wherein the mobile phase has a pH of about 7.5

In some embodiments, the exemplary SEC column may have a material made up of silica particles. In some embodiment, the exemplary silica material is compatible with a pH range of 7.5+/−0.2 and salt concentration of 150 mM NaCl. In some embodiments, the exemplary column may have a pore size in the range of 12.5 to 25 nm. In some embodiments, the internal dimensions of the exemplary column may be 4.6 mm internal diameter (ID), 300 mm length and 4 μm particle size for efficient separation of the two more types of nucleic acids. In some embodiments, a useful column may be Waters Biosuite UHR 125 SEC column, 4.6 mm×30 cm, 4 μm. Part No. 186002161; or Tosoh TSKgel SuperSW30, 4.6 mm ID×30 cm L, Part No. 0018675. In some aspects, the aforementioned columns are used along with an Agilent, Shimazhu, or Waters HPLC system.

In various embodiments, SEC of the present disclosure includes a mobile phase characterized in that it is an aqueous mobile phase. In various embodiments, SEC of the present disclosure includes a mobile phase characterized in that it is an aqueous, salt-based mobile phase. In various embodiments, SEC of the present disclosure includes a mobile phase characterized in that it includes Tris-HCl. In various embodiments, SEC of the present disclosure includes a mobile phase characterized in that it includes about 5 mM to about 50 mM Tris-HCl (e.g., about 20 mM Tris-HCl). In various embodiments, SEC of the present disclosure includes a mobile phase characterized in that it includes NaCl. In various embodiments, SEC of the present disclosure includes a mobile phase characterized in that it includes about 25 mM to about 200 mM NaCl (e.g., about 150 mM NaCl). In various embodiments, SEC of the present disclosure includes a mobile phase characterized in that the SEC separation is carried out at a temperature of about 20° C. to about 40° C. (e.g., about 30° C.). In various embodiments, SEC of the present disclosure includes a mobile phase characterized by a pH between about 7 and about 8. In various embodiments, SEC of the present disclosure includes a mobile phase characterized by a pH between about 7.3 and about 7.7 (e.g., about 7.5).

In various embodiments, SEC of the present disclosure includes a mobile phase characterized in that it includes Tris-HCl and NaCl. In various embodiments, SEC of the present disclosure includes a mobile phase characterized in that it includes about 5 mM to about 50 mM Tris-HCl (e.g., about 20 mM Tris-HCl) and about 25 mM to about 200 mM NaCl (e.g., about 150 mM NaCl).

In various embodiments, SEC of the present disclosure includes a mobile phase characterized in that it includes Tris-HCl and NaCl, and has a pH between about 7 and about 8. In various embodiments, SEC of the present disclosure includes a mobile phase characterized in that it includes about 5 mM to about 50 mM Tris-HCl (e.g., about 20 mM Tris-HCl) and about 25 mM to about 200 mM NaCl (e.g., about 150 mM NaCl), and has a pH between about 7.3 and about 7.7 (e.g., about 7.5).

In various embodiments, SEC of the present disclosure includes a mobile phase characterized in that it includes Tris-HCl and NaCl, and is carried out at a temperature of about 20° C. to about 40° C. In various embodiments, SEC of the present disclosure includes a mobile phase characterized in that it includes about 5 mM to about 50 mM Tris-HCl (e.g., about 20 mM Tris-HCl) and about 25 mM to about 200 mM NaCl (e.g., about 150 mM NaCl), and is carried out at a temperature of about 20° C. to about 40° C. (e.g., about 30° C.).

In various embodiments, SEC of the present disclosure includes a mobile phase characterized in that it includes Tris-HCl and NaCl, has a pH between about 7 and about 8, and is carried out at a temperature of about 20° C. to about 40° C. In various embodiments, SEC of the present disclosure includes a mobile phase characterized in that it includes about 5 mM to about 50 mM Tris-HCl (e.g., about 20 mM Tris-HCl) and about 25 mM to about 200 mM NaCl (e.g., about 150 mM NaCl), has a pH between about 7.3 and about 7.7 (e.g., about 7.5), and is carried out at a temperature of about 20° C. to about 40° C. (e.g., about 30° C.).

In various embodiments, nucleic acids separated by a chromatography substrate and/or column (e.g., an SEC column) are retained in the chromatography substrate and/or column (e.g., an SEC column) with different retention times, exit the chromatography substrate and/or column (e.g., an SEC column) at different times, and/or are detected at different times, any or all of which constitute separation as disclosed herein. In various embodiments, separation can further include physical separation of eluate including different populations of nucleic acids and/or detection of different populations of nucleic acids.

In various embodiments, SEC of the present disclosure separates a first nucleic acid and a second nucleic acid by at least 1, 2, 3, 4, 5, 6, 7, 8, 9, or 10 in a resulting chromatographic profile, e.g., at a flow rate of 0.3 mL/min. In various embodiments, SEC of the present disclosure separates a first nucleic acid and a second nucleic acid by at least about 1 minute in a resulting chromatographic profile, e.g., at a flow rate of 0.3 mL/min. In various embodiments, SEC of the present disclosure separates a first nucleic acid and a second nucleic acid by at least about 1.5 in a resulting chromatographic profile, e.g., at a flow rate of 0.3 mL/min. Separation of a first nucleic acid a second a second nucleic acid can be calculated according to various means known in the art, e.g., based on peak or based on peak width at half-height.

In various embodiments, methods of the present disclosure include ion pair chromatography (e.g., ion pair reverse phase HPLC) using a substrate and/or column characterized by one or more particular characteristics provided herein. In various embodiments, ion pair chromatography of the present disclosure includes contacting a nucleic acid extract with an ion pair chromatography substrate and/or column recognized for use in separation of nucleic acids, e.g., DNA (e.g., single-stranded and/or double-stranded DNA) and/or RNA (e.g., single-stranded and/or double-stranded RNA). In various embodiments, ion pair chromatography of the present disclosure includes contacting a nucleic acid extract with an ion pair chromatography substrate and/or column characterized by hydrophobic particles.

In various embodiments, ion pair chromatography of the present disclosure (e.g., ion pair reverse phase HPLC) includes contacting a nucleic acid extract with an ion pair chromatography substrate and/or column characterized by particles having a mean, median, and/or modal size between about 2 μm and about 6 μm, e.g., having a size of about 2 μm, about 3 μm, about 4 μm, about 5 μm, or about 6 μm. In various embodiments, ion pair chromatography of the present disclosure includes contacting a nucleic acid extract with an ion pair chromatography substrate and/or column characterized by particles having a mean, median, and/or modal size between about 2 μm and about 6 μm, between about 2 μm and about 5 μm, between about 2 μm and about 4 μm, between about 2 μm and about 3 μm, between about 3 μm and about 6 μm, between about 3 μm and about 5 μm, between about 3 μm and about 4 μm, between about 4 μm and about 6 μm, or between about 4 μm and about 5 μm, In various embodiments, ion pair chromatography of the present disclosure includes contacting a nucleic acid extract with an ion pair chromatography substrate and/or column characterized by particles having a mean, median, and/or modal size of about 4 μm

In various embodiments, ion pair chromatography of the present disclosure (e.g., ion pair reverse phase HPLC) includes contacting a nucleic acid extract with an ion pair chromatography substrate and/or column characterized by particles having a mean, median, and/or modal pore size of about 100 Å to about 3000 Å, e.g., having a size of about 200 Å, about 300 Å, about 400 Å, about 500 Å, about 600 Å, about 700 Å, about 800 Å, about 900 Å, about 1000 Å, about 1250 Å, about 1500 Å, about 1750 Å, about 2000 Å, about 2250 Å, about 2500 Å, about 2750 Å, or about 3000 Å. In various embodiments, ion pair chromatography of the present disclosure includes contacting a nucleic acid extract with an ion pair chromatography substrate and/or column characterized by particles having a mean, median, and/or modal pore size of about 100 Å to about 3000 Å, about 100 Å to about 2500 Å, about 100 Å to about 2000 Å, about 100 Å to about 1500 Å, about 100 Å to about 1000 Å, about 100 Å to about 500 Å, about 300 Å to about 3000 Å, about 300 Å to about 2500 Å, about 300 Å to about 2000 Å, about 300 Å to about 1500 Å, about 300 Å to about 1000 Å, about 300 Å to about 500 Å, about 500 Å to about 3000 Å, about 500 Å to about 2500 Å, about 500 Å to about 2000 Å, about 500 Å to about 1500 Å, about 500 Å to about 1000 Å, about 1000 Å to about 3000 Å, about 1000 Å to about 2500 Å, about 1000 Å to about 2000 Å, or about 1000 Å to about 1500 Å. In various embodiments, ion pair chromatography of the present disclosure (e.g., ion pair reverse phase HPLC) includes contacting a nucleic acid extract with an ion pair chromatography substrate and/or column characterized by particles having a mean, median, and/or modal pore size of about 1000 Å to about 2000 Å.

In various embodiments, ion pair chromatography of the present disclosure (e.g., ion pair reverse phase HPLC) includes contacting a nucleic acid extract with an ion pair chromatography column characterized by a diameter of about 1 mm to about 4 mm, e.g., about 1 mm, about 2 mm, about 3 mm, or about 4 mm. In various embodiments, ion pair chromatography of the present disclosure includes contacting a nucleic acid extract with an ion pair chromatography column characterized by a diameter of about 1 mm to about 4 mm, 1.5 mm to about 4 mm, 2 mm to about 4 mm, 2.5 mm to about 4 mm, 3 mm to about 4 mm, 3.5 mm to about 4 mm, 1 mm to about 4 mm, 1.5 mm to about 3 mm, 2 mm to about 3 mm, 2.5 mm to about 3 mm, about 1 mm to about 2.5 mm, about 1.5 mm to about 2.5 mm, about 2 mm to about 2.5 mm. about 1 mm to about 2 mm, or about 1.5 mm to about 2 mm. In various embodiments, ion pair chromatography of the present disclosure includes contacting a nucleic acid extract with an ion pair chromatography column characterized by a diameter of about 2.1 mm.

In various embodiments, ion pair chromatography of the present disclosure (e.g., ion pair reverse phase HPLC) includes contacting a nucleic acid extract with an ion pair chromatography column characterized by a length of about 10 mm to about 300 mm, e.g., about 25 mm, about 50 mm, about 75 mm, about 100 mm, about 125 mm, about 150 mm, about 175 mm, about 200 mm, about 225 mm, about 250 mm, about 275 mm, or about 300 mm. In various embodiments, ion pair chromatography of the present disclosure includes contacting a nucleic acid extract with an ion pair chromatography column characterized by a length of about 25 mm to about 300 mm, about 50 mm to about 300 mm, about 75 mm to about 300 mm, about 100 mm to about 300 mm, about 125 mm to about 300 mm, about 150 mm to about 300 mm, about 175 mm to about 300 mm, about 200 mm to about 300 mm, about 225 mm to about 300 mm, about 250 mm to about 300 mm, or about 275 mm to about 300 mm. In various embodiments, ion pair chromatography of the present disclosure includes contacting a nucleic acid extract with an ion pair chromatography column characterized by a length of about 25 mm to about 150 mm, about 50 mm to about 150 mm, about 75 mm to about 150 mm, about 100 mm to about 150 mm, or about 125 mm to about 150 mm. In various embodiments, ion pair chromatography of the present disclosure includes contacting a nucleic acid extract with an ion pair chromatography column characterized by a length of about 100 mm.

In various embodiments, ion pair chromatography of the present disclosure (e.g., ion pair reverse phase HPLC) includes an ion pairing agent. Ion pairing agents can include both an ionic functional group and a hydrophobic portion, such as a hydrocarbon chain and/or a lipophilic alkyl chain. Ion pairing agents can be sulfonic acid derivatives such as hexane-, heptane-, octane-sulfonic acids, quaternary ammonium salts such as tetramethyl- or tetrabutylammonium hydroxide, or volatile agents such as trifluoroacetic acid and triethylamine. In certain exemplary embodiments, an ion pairing agent can be triethylamine, tripropylamine, hexylamine, N,N-dimethylbutylamine, dibutylamine, N,N-diisopropylethylamine) or hexafluoro-2-propanol. In certain exemplary embodiments, an ion pairing agent can be trimethylamine or hexylamine. In certain exemplary embodiments, an ion pairing agent can be dibutylammonium acetate. In certain exemplary embodiments, an ion pairing agent can be triethylammonium acetate. In certain exemplary embodiments, an ion pairing agent can be dibutylammonium acetate and triethylammonium acetate.

In various embodiments, ion pair chromatography of the present disclosure includes contacting a nucleic acid extract with an ion pair chromatography substrate and/or column comprising silica, optionally wherein the substrate and/or column includes silica particles. In various embodiments, ion pair chromatography of the present disclosure includes contacting a nucleic acid extract with an ion pair chromatography substrate that comprises particles to which the ion pairing agent can bind. Those of skill in the art will be familiar with ion pair chromatography substrates and columns, and will be able to select a suitable column for use in methods and compositions provided herein based on the present disclosure.

In various embodiments a counter ion is also provided, e.g., in a mobile phase. In various embodiments, ion pair chromatography of the present disclosure (e.g., ion pair reverse phase HPLC) includes a mobile phase characterized in that it includes ion pairing agent, e.g., 10 mM to 300 mM ion pairing agent. In various embodiments, a mobile phase can include, e.g., about 25 mM, about 50 mM, about 75 mM, about 100 mM, about 125 mM, about 150 mM, about 175 mM, about 200 mM, about 225 mM, about 250 mM, about 275 mM, or about 300 mM ion pairing agent. In various embodiments, a mobile phase can include, e.g., about 25 mM to about 300 mM ion pairing agent, about 50 mM to about 300 mM ion pairing agent, about 75 mM to about 300 mM ion pairing agent, about 100 mM to about 300 mM ion pairing agent, about 150 mM to about 300 mM ion pairing agent, about 200 mM to about 300 mM ion pairing agent, about 25 mM to about 250 mM ion pairing agent, about 50 mM to about 250 mM ion pairing agent, about 75 mM to about 250 mM ion pairing agent, about 100 mM to about 250 mM ion pairing agent, about 150 mM to about 250 mM ion pairing agent, about 200 mM to about 250 mM ion pairing agent, about 25 mM to about 200 mM ion pairing agent, about 50 mM to about 200 mM ion pairing agent, about 75 mM to about 200 mM ion pairing agent, about 100 mM to about 200 mM ion pairing agent, about 150 mM to about 200 mM ion pairing agent, about 25 mM to about 150 mM ion pairing agent, about 50 mM to about 150 mM ion pairing agent, about 75 mM to about 150 mM ion pairing agent, or about 100 mM to about 150 mM ion pairing agent.

In various embodiments a counter ion is also provided, e.g., in a mobile phase. In various embodiments, ion pair chromatography of the present disclosure (e.g., ion pair reverse phase HPLC) includes a mobile phase characterized in that it includes dibutylammonium acetate, e.g., 10 mM to 300 mM dibutylammonium acetate. In various embodiments, a mobile phase can include, e.g., about 25 mM, about 50 mM, about 75 mM, about 100 mM, about 125 mM, about 150 mM, about 175 mM, about 200 mM, about 225 mM, about 250 mM, about 275 mM, or about 300 mM dibutylammonium acetate. In various embodiments, a mobile phase can include, e.g., about 25 mM to about 300 mM dibutylammonium acetate, about 50 mM to about 300 mM dibutylammonium acetate, about 75 mM to about 300 mM dibutylammonium acetate, about 100 mM to about 300 mM dibutylammonium acetate, about 150 mM to about 300 mM dibutylammonium acetate, about 200 mM to about 300 mM dibutylammonium acetate, about 25 mM to about 250 mM dibutylammonium acetate, about 50 mM to about 250 mM dibutylammonium acetate, about 75 mM to about 250 mM dibutylammonium acetate, about 100 mM to about 250 mM dibutylammonium acetate, about 150 mM to about 250 mM dibutylammonium acetate, about 200 mM to about 250 mM dibutylammonium acetate, about 25 mM to about 200 mM dibutylammonium acetate, about 50 mM to about 200 mM dibutylammonium acetate, about 75 mM to about 200 mM dibutylammonium acetate, about 100 mM to about 200 mM dibutylammonium acetate, about 150 mM to about 200 mM dibutylammonium acetate, about 25 mM to about 150 mM dibutylammonium acetate, about 50 mM to about 150 mM dibutylammonium acetate, about 75 mM to about 150 mM dibutylammonium acetate, or about 100 mM to about 150 mM dibutylammonium acetate.

In various embodiments a counter ion is also provided, e.g., in a mobile phase. In various embodiments, ion pair chromatography of the present disclosure (e.g., ion pair reverse phase HPLC) includes a mobile phase characterized in that it includes triethylammonium acetate, e.g., 10 mM to 300 mM triethylammonium acetate. In various embodiments, a mobile phase can include, e.g., about 25 mM, about 50 mM, about 75 mM, about 100 mM, about 125 mM, about 150 mM, about 175 mM, about 200 mM, about 225 mM, about 250 mM, about 275 mM, or about 300 mM triethylammonium acetate. In various embodiments, a mobile phase can include, e.g., about 25 mM to about 300 mM triethylammonium acetate, about 50 mM to about 300 mM triethylammonium acetate, about 75 mM to about 300 mM triethylammonium acetate, about 100 mM to about 300 mM triethylammonium acetate, about 150 mM to about 300 mM triethylammonium acetate, about 200 mM to about 300 mM triethylammonium acetate, about 25 mM to about 250 mM triethylammonium acetate, about 50 mM to about 250 mM triethylammonium acetate, about 75 mM to about 250 mM triethylammonium acetate, about 100 mM to about 250 mM triethylammonium acetate, about 150 mM to about 250 mM triethylammonium acetate, about 200 mM to about 250 mM triethylammonium acetate, about 25 mM to about 200 mM triethylammonium acetate, about 50 mM to about 200 mM triethylammonium acetate, about 75 mM to about 200 mM triethylammonium acetate, about 100 mM to about 200 mM triethylammonium acetate, about 150 mM to about 200 mM triethylammonium acetate, about 25 mM to about 150 mM triethylammonium acetate, about 50 mM to about 150 mM triethylammonium acetate, about 75 mM to about 150 mM triethylammonium acetate, or about 100 mM to about 150 mM triethylammonium acetate.

In certain embodiments, ion pair chromatography of the present disclosure (e.g., ion pair reverse phase HPLC) includes a mobile phase characterized in that it includes dibutylammonium acetate and triethylammonium acetate. In certain exemplarily embodiments, ion pair chromatography of the present disclosure (e.g., ion pair reverse phase HPLC) includes a mobile phase characterized in that it includes about 50 mM dibutylammonium acetate and about 100 mM triethylammonium acetate. In various embodiments, ion pair chromatography of the present disclosure (e.g., ion pair reverse phase HPLC) includes two or more mobile phases as set forth here. For example, in some embodiments, ion pair chromatography of the present disclosure (e.g., ion pair reverse phase HPLC) includes a first mobile phase characterized in that it includes about 50 mM dibutylammonium acetate and about 100 mM triethylammonium acetate and a second mobile phase characterized in that it includes about 50 mM dibutylammonium acetate and about 100 mM triethylammonium acetate. In certain embodiments, ion pair chromatography of the present disclosure (e.g., ion pair reverse phase HPLC) can be carried out at any pH (i.e., from 0 to 14). In various embodiments, ion pair chromatography can be carried out at a pH between about 7 and about 8.

In certain embodiments, ion pair chromatography of the present disclosure (e.g., ion pair reverse phase HPLC) can be carried out at any temperature. In various embodiments, ion pair chromatography of the present disclosure (e.g., ion pair reverse phase HPLC) can be carried out at a temperature of about 20° C. to about 80° C. (e.g., about 65° C.).

In various embodiments, nucleic acids separated by ion pair chromatography are retained in the chromatography substrate and/or column with different retention times, exit the chromatography substrate and/or column at different times, and/or are detected at different times, any or all of which constitute separation or purification as disclosed herein. In various embodiments, separation can further include physical separation of eluate including different populations of nucleic acids and/or detection of different populations of nucleic acids.

In various embodiments, ion pair chromatography of the present disclosure separates a first nucleic acid and a second nucleic acid by at least 1, 2, 3, 4, 5, 6, 7, 8, 9, or 10 in a resulting chromatographic profile, e.g., at a flow rate of 0.45 mL/min. In various embodiments, ion pair chromatography of the present disclosure separates a ~100mer RNA from a ~1000mer RNA by at least or about 1, 2, or 3 (e.g, by at least or about 1, 1.1, 1.2, 1.3, 1.4, 1.5, 1.6, 1.7, 1.8, 1.9, 2.0, 2.1, 2.2., 2.3, 2.4, 2.5, 2.6, 2.7, 2.8, 2.9, or 3.0), e.g., at a flow rate of 0.45 mL/min. In various embodiments, ion pair chromatography of the present disclosure separates a ~100mer RNA from a ~4500mer RNA by at least or about 1, 2, 3, 4 or 5 (e.g, by at least or about 1, 1.1, 1.2, 1.3, 1.4, 1.5, 1.6, 1.7, 1.8, 1.9, 2.0, 2.1, 2.2., 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0), e.g., at a flow rate of 0.45 mL/min. In various embodiments, ion pair chromatography of the present disclosure separates a ~1000mer RNA from a ~4500mer RNA by at least or about 1 or 2 (e.g, by at least or about 1, 1.1, 1.2, 1.3, 1.4, 1.5, 1.6, 1.7, 1.8, 1.9, or 2.0), e.g., at a flow rate of 0.45 mL/min. Separation of a first nucleic acid a second a second nucleic acid can be calculated according to various means known in the art, e.g., based on peak or based on peak width at half-height.

### Detection of Nucleic Acids and Determination of Nucleic Acid Concentrations

The present disclosure includes that methods provided herein for assessment of lipid nucleic acid assembly compositions can include determining the absolute and/or relative concentration of one or more separated nucleic acids (e.g., nucleic acids of a lipid nucleic acid assembly composition from which a nucleic acid extract was prepared and subjected to chromatography, in accordance with the present disclosure). Chromatography of the present disclosure can include a variety of detectors for detecting nucleic acids, e.g., in column eluate, which detection can provide, among other things, the absolute and/or relative concentration of one or more nucleic acids, and/or an absolute and/or relative total nucleic acid concentration.

As those of skill in the art will appreciate, any of a variety of detectors can be suitable for use in the detection of nucleic acids during or in connection with chromatography. To provide one particular example, in some embodiments, nucleic acids can be detected by measurement of absorbance. In some embodiments, absorbance can be measured at 260 nm and/or at 260 nm and 280 nm. In some embodiments, absorbance can be measured using a UV detector or spectrophotometer. In some embodiments, a UV detector can be a fixed wavelength UV detector, variable wavelength UV detector (VWD), or photodiode array UV detector (DAD). In certain examples, nucleic acids can be detected by use of nucleic acid-binding dyes and measurement of fluorescence, by analysis of eluate electrophoresis (e.g., on an agarose gel), and/or by PCR (e.g., quantitative PCR, reverse-transcription PCR, and/or quantitative reverse-transcription PCR) for amplification of nucleic acid sequences of interest.

In some embodiments, nucleic acid concentration and/or purity is assessed using UV absorbance or UV spectroscopy, in which the absorbance of a diluted RNA sample is measured at 260 nm, and/or at 260 nm and 280 nm. The Beer-Lambert Law allows the concentration of nucleic acids in a sample to be calculated based on the amount of light absorbed at 260 nm: A=εbc, where A is absorbance (also known as optical density), ε is the extinction coefficient, b is the path length of the sample cuvette, and c is the concentration of the compound in a solution.

Those of skill in the art will appreciate that calibration curves can be designed to provide calibration for detection of an analyte of interest over a concentration range of interest (e.g., a nucleic acid, RNA, sgRNA, mRNA, total nucleic acid, and/or total RNA concentration disclosed herein). Those of skill in the art will further appreciate from the present disclosure that a calibration curve is a form of reference or control, and as such is subjected to same or comparable analysis processes or conditions as a tested composition (e.g., a lipid nucleic acid assembly composition or a nucleic acid extract) to which it is compared (e.g., same or comparable chromatography and/or same or comparable detection, e.g., detection by absorbance at 260 nm). The present disclosure includes, for example, that in various embodiments a calibration curve or curves can be produced using a dilution series of known concentrations of nucleic acid (e.g., known concentrations of mRNA and/or known concentrations of sgRNA), e.g., where the dilution series represents a linear series of concentration values. In certain embodiments, a calibration curve or curves can be produced using a dilution series of known concentrations of a reference mRNA and/or of a reference sgRNA, e.g., of an mRNA and/or an sgRNA known or expected to be present in a lipid nucleic acid assembly composition, e.g., where the dilution series represents a linear series of concentration values. In various embodiments, concentration of a first nucleic acid (e.g., an mRNA present in a lipid nucleic acid assembly composition or nucleic acid extract) can be determined in accordance with a calibration curve of a first reference nucleic acid (e.g., a reference mRNA) and concentration of a second nucleic acid (e.g., an sgRNA present in a lipid nucleic acid assembly composition or nucleic acid extract) can be determined in accordance with a calibration curve of a second reference nucleic acid (e.g., a reference sgRNA). In various embodiments, e.g., embodiments in which nucleic acid present in a lipid nucleic acid assembly composition consists or consists essentially of a first nucleic acid and a second nucleic acid, total nucleic acid concentration (e.g., total RNA concentration) can be determined as the sum of the concentration of a first nucleic acid (e.g., an mRNA) and a second nucleic acid (e.g., an sgRNA).

In various embodiments, a series of reference dilutions of a calibration curve include, in each dilution, both a concentration of a first reference nucleic acid and a concentration of a second reference nucleic acid, e.g., where each dilution in the series has, or is intended to have, the same, or about the same, ratio of first reference nucleic acid to second reference nucleic acid (a “constant” or “maintained” ratio). In various embodiments, the constant or maintained ratio is, or is intended to be, the same, or about the same, as the actual or expected ratio of a first nucleic acid and a second nucleic acid in a lipid nucleic acid assembly composition and/or nucleic acid extract. In various embodiments, a series of reference dilutions of a calibration curve, each including both a concentration of a first nucleic acid and a concentration of a second nucleic acid, can have a range of total nucleic acid concentrations. In various embodiments, the total nucleic acid concentrations of reference dilutions, each including both a concentration of a first nucleic acid and a concentration of a second nucleic acid, represents a linear series of values.

Those of skill in the art will appreciate that the concentration range of a dilution series of a calibration curve can be selected to encompass a relevant range, and that the relevant range can be determined by a user through dilution or concentration of a nucleic acid extract. In various embodiments, a nucleic acid extract and calibration curve are prepared and/or selected such that the nucleic acid extract has a concentration (e.g., an mRNA concentration, sgRNA concentration, and/or total concentration) at or about the midpoint of a calibration curve, e.g., based on measured or expected values. Solely to provide exemplary embodiments, a calibration curve of a reference mRNA molecule can represent a range of concentrations that falls between and/or includes a lower bound of about 12 μg/mL and an upper bound of about 60 μg/mL, or that falls between and/or includes a lower bound of about 12 μg/mL and an upper bound of about 36 μg/mL. Solely to provide exemplary embodiments, a calibration curve of a reference sgRNA molecule can represent a range of concentrations that falls between and/or includes a lower bound of about 8 μg/mL and an upper bound of about 40 μg/mL, or that falls between and/or includes a lower bound of about 8 μg/mL and an upper bound of about 24 μg/mL. In various embodiments, each dilution of a dilution series can include both a reference mRNA and a reference sgRNA, where the mRNA and sgRNA are present in each dilution in the same ratio (e.g., 2:1 or 3:2) and in various embodiments can have total RNA concentrations that fall between and/or includes a lower bound of about 20 μg/mL and an upper bound of about 100 μg/mL, or that fall between and/or includes a lower bound of about 20 μg/mL and an upper bound of about 60 μg/mL.

Using the Beer-Lambert Law, a calibration curve can be obtained by preparing various diluted nucleic acid samples in nuclease-free water, and plotting the absorbance measured at 260 nm of the diluted nucleic acid samples against their respective concentrations. For example, using the Beer-Lambert Law, an A260 reading of 1.0 is equivalent to ~40 μg/ml single-stranded RNA. In some embodiments, the A260/A280 ratio can be used to assess DNA/RNA purity. For example, an A260/A280 ratio of 1.8-2.1 is indicative of highly purified RNA (e.g., A260/A280 ratio is 1.85 for nuclease-free water (pH 6-7)).

### Applications

Compositions and methods provided herein can be used to purify and/or separate one or more nucleic acids (e.g., a first and second nucleic acid, e.g., an mRNA and an sgRNA) present in a lipid nucleic acid assembly composition. Compositions and methods provided herein can be used to determine the concentration of each of one or more (e.g., two) nucleic acids (e.g., a first and second nucleic acid, e.g., an mRNA and an sgRNA) present in a lipid nucleic acid assembly composition. Compositions and methods provided herein can be used to determine the ratios of each of two or more (e.g., two) nucleic acids (e.g., a first and second nucleic acid, e.g., an mRNA and an sgRNA) present in a lipid nucleic acid assembly composition. Compositions and methods provided herein can be used to determine the absolute and/or relative total concentration of nucleic acid present in a lipid nucleic acid assembly composition.

The present disclosure includes that information such as absolute and/or relative nucleic acid concentrations and/or ratios determined according to the compositions and methods set forth in the present disclosure can be used for a variety of purposes, including in research and pharmaceutical preparation. For example, compositions and methods disclosed herein can be used to determine the absolute and/or relative concentrations of nucleic acids in a lipid nucleic acid assembly composition, ratio of nucleic acids in lipid nucleic acid assembly composition, and/or total nucleic acid concentration in a lipid nucleic acid assembly composition, e.g., where the nucleic acids present in a lipid nucleic acid assembly composition include, consist, or consist essentially of two nucleic acids, e.g., an mRNA and an sgRNA. In various embodiments, absolute and/or relative concentrations of nucleic acids in a lipid nucleic acid assembly composition, ratio of nucleic acids in lipid nucleic acid assembly composition, and/or total nucleic acid concentration in a lipid nucleic acid assembly composition can be compared to a reference such as a reference value that characterizes a therapeutic product. In various embodiments, the lipid nucleic acid assembly composition is a drug product or drug substance, e.g., a drug product or drug substance that includes nucleic acids that include, consist, or consist essentially of an mRNA and an sgRNA. In some embodiments, a drug product or drug substance is formulated for pharmaceutical use if the drug product or drug substance, or a lipid nucleic acid assembly composition derived therefrom, is determined to have an absolute and/or relative concentration of each of one or more (e.g., two) nucleic acids, ratio of two nucleic acids, and/or total nucleic acid concentration that is the same as, or substantially the same as (e.g., that is within 99%, 98%, 97%, 96%, 95%, or 90% of), that of a reference value that characterizes a drug product. In some embodiments, a drug product or drug substance is not formulated for pharmaceutical use if the drug product or drug substance, or a lipid nucleic acid assembly composition derived therefrom, is determined to have an absolute and/or relative concentration of each of one or more (e.g., two) nucleic acids, ratio of nucleic acids, and/or total nucleic acid concentration that is not the same as, or that is not substantially the same as (e.g., that is not within 99%, 98%, 97%, 96%, 95%, or 90% of), that of a reference value that characterizes a therapeutic product.

### Kits

The present disclosure includes, among other things, a kit that includes an SEC substrate and instructions for use thereof in separating two or more nucleic acids present in a lipid nucleic acid assembly composition. The present disclosure includes, among other things, a kit that includes an SEC substrate and one or both of (i) at least one reagent for SEC, such as a mobile phase solution and/or (ii) a reagent for ethanol precipitation, isopropyl alcohol precipitation, butanol precipitation, or acetonitrile precipitation of nucleic acid from a lipid nucleic acid assembly composition. The present disclosure includes, among other things, a kit that includes an SEC substrate and one or both of (i) at least one reagent for SEC, such as a mobile phase solution and/or (ii) a reagent for ethanol precipitation, isopropyl alcohol precipitation, butanol precipitation, or acetonitrile precipitation of nucleic acid from a lipid nucleic acid assembly composition, together with instructions for use thereof in separating two or more nucleic acids present in a lipid nucleic acid assembly composition. Various such kits can further include instructions for determining absolute and/or relative total nucleic acid concentration, individual nucleic acid concentrations, and/or concentration ratios. In various such kits the SEC substrate is, or is included in, an SEC column. In various such kits, an SEC substrate, reagent for SEC, SEC mobile phase solution, and/or a reagent for ethanol solutions are as described elsewhere herein.

In at least one aspect, the present disclosure provides a method of separating nucleic acid present in a lipid nucleic acid assembly composition, the method including: a) preparing a nucleic acid extract from the lipid nucleic acid assembly composition, wherein the preparation of the nucleic acid extract includes ethanol precipitation of the nucleic acid from the lipid nucleic acid assembly composition; and b) subjecting the nucleic acid extract to SEC, wherein the SEC separates the nucleic acid. In certain embodiments, the method further includes determining the total concentration of the nucleic acid present in the lipid nucleic acid assembly composition.

In at least one aspect, the present disclosure provides a kit for separating nucleic acid present in a lipid nucleic acid assembly composition, the kit including a SEC substrate and at least one reagent for ethanol precipitation of the nucleic acid from the lipid nucleic acid assembly composition, optionally wherein the kit is for separating two or more nucleic acids, optionally wherein the two or more nucleic acid molecules are or include a gRNA and an mRNA. In certain embodiments, the SEC substrate is, or is present in, an SEC column. In certain embodiments, the SEC column has an internal diameter of 3 mm to 6 mm, optionally an internal diameter of about 4 mm to about 5 mm, optionally an internal diameter of about 4.6 mm internal diameter. In certain embodiments, the SEC column has a length of about 100 mm to about 400 mm, optionally a length of about 300 mm length. In certain embodiments, the SEC substrate and/or SEC column has a pore size of about 5 nm to about 50 nm, optionally wherein the pore size is about 12.5 nm to about 25 nm. In certain embodiments, the SEC substrate and/or SEC column has a particle size of about 3 μm to about 5 μm, optionally a particle size of about 4 μm. In certain embodiments, the SEC substrate and/or SEC column includes silica, optionally wherein the SEC substrate includes silica particles. In certain embodiments, the SEC substrate and/or SEC column is compatible with a mobile phase having a pH of about 7 to about 8, optionally a mobile phase having a pH of about 7.3 to about 7.7, optionally a mobile phase having a pH of about 7.5. In certain embodiments, the SEC substrate and/or SEC column is compatible with a mobile phase including about 25 mM to about 200 mM NaCl, optionally a mobile phase including about 150 mM NaCl.

In certain embodiments of kits provided herein, the kit includes a dilution series of one or more reference nucleic acids, optionally wherein the kit includes a dilution series of a reference gRNA molecule and/or a reference mRNA molecule. In certain embodiments of kits provided herein, the kit includes instructions for preparation of a nucleic acid extract by a process including ethanol precipitation. In certain embodiments of kits provided herein, the kit includes instructions for separating the two or more nucleic acids by SEC, optionally wherein the two or more nucleic acids include or consist of RNA molecules, optionally wherein the RNA molecules include or consist of a gRNA molecule and an mRNA molecule. In certain embodiments of kits provided herein, the kit includes instructions for determining the concentration of the two more nucleic acids and/or total nucleic acid concentration, optionally wherein the two or more nucleic acids include or consist of RNA molecules, optionally wherein the RNA molecules include or consist of a gRNA molecule and an mRNA molecule.

In at least one aspect, the present disclosure provides a kit for separating nucleic acid present in a lipid nucleic acid assembly composition, the kit including a ion pair chromatography substrate and at least one reagent for ethanol precipitation of the nucleic acid from the lipid nucleic acid assembly composition, optionally where the kit is for separating two or more nucleic acids, optionally where the two or more nucleic acid molecules are or include a gRNA and an mRNA, and/or for separating three or more nucleic acids, optionally where the three or more nucleic acid molecules are or include a gRNA, a first mRNA, and a second mRNA. In certain embodiments, the ion pair chromatography substrate is, or is present in, an ion pair chromatography column. In certain embodiments, the ion pair chromatography column has an internal diameter of 1 mm to 4 mm, optionally an internal diameter of about 1.5 mm to about 2.5 mm, optionally an internal diameter of about 2.1 mm internal diameter. In certain embodiments, the ion pair chromatography column has a length of about 50 mm to about 400 mm, optionally a length of about 100 mm length. In certain embodiments, the ion pair chromatography substrate and/or ion pair chromatography column has a pore size of about 100 Å to about 3000 Å, optionally where the pore size is about 1000 Å to about 2000 Å. In certain embodiments, the ion pair chromatography substrate and/or ion pair chromatography column has a particle size of about 3 μm to about 5 μm, optionally a particle size of about 4 μm. In certain embodiments, the ion pair chromatography substrate and/or ion pair chromatography column includes silica, optionally where the ion pair chromatography substrate includes silica particles. In certain embodiments, the ion pair chromatography substrate and/or ion pair chromatography column is compatible with a mobile phase including dibutylammonium acetate and/or triethylammonium acetate. In certain embodiments, the kit includes a dilution series of one or more reference nucleic acids, optionally where the kit includes a dilution series of a reference gRNA molecule, a first reference mRNA molecule, and/or a second reference mRNA molecule. In certain embodiments, the kit includes instructions for preparation of a nucleic acid extract by a process including ethanol precipitation. In certain embodiments, the kit includes instructions for separating the two or more nucleic acids. or three or more nucleic acids, by ion pair chromatography, optionally where the nucleic acids include RNA molecules, optionally where the RNA molecules include a gRNA molecule, a first mRNA molecule, and/or a second mRNA molecule. In certain embodiments, the kit includes instructions for determining the concentration of the two more nucleic acids, three or more nucleic acids, and/or total nucleic acid concentration, optionally where the nucleic acids include RNA molecules, optionally where the RNA molecules include a gRNA molecule, a first mRNA molecule, and/or a second mRNA molecule.

### EXAMPLES

The present Examples provide exemplary protocols and reagents useful in separating, and/or determining the absolute and/or relative concentration of, nucleic acids in a lipid nucleic acid assembly composition as set forth in the present disclosure. The present Examples provide for determination of RNA concentration from a lipid nucleic acid assembly composition in which mRNA and sgRNA are encapsulated in LNPs. Without wishing to be bound by any particular scientific theory, LNPs are de-formulated by ethanol precipitation, allowing release of RNA. Extracted RNA is then subjected to SEC, allowing for separation of mRNA and sgRNA based on their relative size difference. Concentrations are quantified by UV detection at 260 nm.

### Example 1: Nucleic Acid Extraction

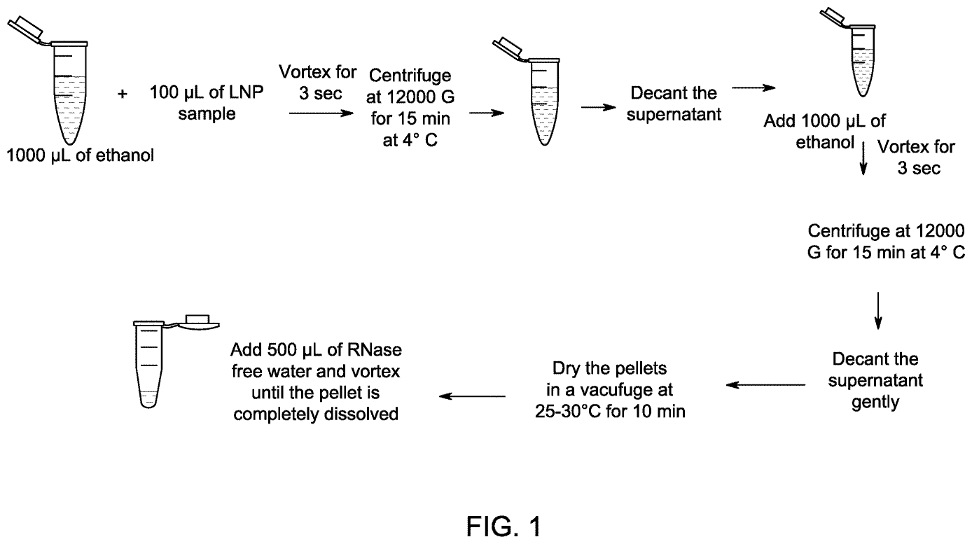

This Example provides exemplary steps for extraction of nucleic acids (here, RNA) from a lipid nucleic acid assembly composition in which the nucleic acids are encapsulated in LNPs. Exemplary steps for extraction of nucleic acids are also provided in FIG. 1.

1) 1000 μL of Ethanol (Absolute Ethanol; 200 Proof) was added to a suitable RNase-Free tube for each lipid nucleic acid assembly composition.

2) 100 μL of LNP sample was added to each ethanol tube and vortexed for 3 seconds.

3) Tubes were centrifuged at 12,000 RCF for 15 minutes at 4° C. After centrifugation, a white RNA pellet was observed at the bottom of the tube.

4) The supernatant ethanol solution was decanted and discarded by gently inverting the tube without disturbing the pellet at the bottom of the tube.

5) An additional 1000 μL of Ethanol was added to each tube. The tube was vortexed for 3 seconds and then centrifuged at 12,000 RCF for 15 minutes at 4° C. After centrifugation, a white RNA pellet was observed at the bottom of the tube.

6) The supernatant ethanol solution was decanted and discarded by gently inverting the tube. A small amount of ethanol may still be observed after decanting. The pellets were dried in a vacufuge at 25-30° C. for 10 minutes±5 minutes to make sure no residual ethanol remains in the vial. If so, additional drying process can be performed.

7) 500 μL of RNase Free water was added to each tube and vortexed until the pellet was completely dissolved.

8) The tubes were spun for 10 seconds to collect the solution on the bottom of the tube.

The extraction procedure diluted the sample nucleic acid concentration by 5-fold. For example, if the sample was provided at the nominal concentration of 1.5 mg/mL total RNA, the extracted sample should have 300 μg/mL of total RNA, provided 100% recovery during extraction. Dilution can be adjusted to produce a nucleic acid extract having a nucleic acid concentration within the range of a desired calibration curve, and preferably at or near the midpoint of the calibration curve. Nucleic acid extracts were further subjected to denaturation and cooling prior to transfer to HPLC vials for separation. These steps included heating to 70° C. for 10 minutes, followed by cooling to 4° C. In the present Examples, the calibration curve range was 12-36 μg/mL mRNA and 8-24 μg/mL sgRNA (i.e., 20-60 μg/mL for total RNA).

### Example 2: Size Exclusion Chromatography of Nucleic Acid Extract

The present Example provides reagents and steps for SEC separation of nucleic acids in a nucleic acid extract prepared according to Example 1, and determination of nucleic acid concentration.

One step of the SEC protocol provided herein was preparation of a dilution series for a calibration curve. The present example includes a calibration curve in which a stock solution was used to prepare a series of five dilutions (Working Standards, “WS”), each dilution including both reference mRNA and reference sgRNA as shown in the below table.

TABLE 1
Exemplary Dilution Series for Calibration Curve

| | | RNase- | Final | | | |
|---|---|---|---|---|---|---|
| | Stock | free | Volume | [mRNA] | [sgRNA] | [Total RNA] |
| Dilution | (μL) | Water | (μL) | (μg/mL) | (μg/mL) | (μg/mL) |
| Stock | 500 | 0 | 500 | 60 | 40 | 100^{b} |
| WS 5 | 300 | 200 | 500 | 36 | 24 | 60 |
| WS 4 | 300 | 300 | 600 | 30 | 20 | 50 |
| WS 3 | 200 | 300 | 500 | 24 | 16 | 40 |
| WS 2 | 150 | 350 | 500 | 18 | 12 | 30 |
| WS 1 | 150 | 600 | 750 | 12 | 8 | 20 |

One step of the SEC protocol provided herein was preparation of the SEC mobile phase. The SEC mobile phase was prepared as 20 mM Tris-HCl, 150 mM NaCl in Water, pH 7.5.

SEC was performed using a Waters BioSuite UHR SEC, 125 Å, 4 μm, 4.6×300 mm column (Catalog 186002161) and an HPLC system with UV Detection (260 nm) or equivalent equipped with: autosampler (capable to maintain 5±2° C.), column heater (capable to maintain 30° C.) and capable to deliver 10 μL injection volume. Standard methodologies were applied for column preparation including equilibration and conditioning, using WS3 as a system readiness check. Operating conditions and an exemplary injection sequence are provided below, showing triplicate testing of each sample.

TABLE 2
Operating Conditions

| | |
|---|---|
| Mobile Phase A: | 20 mM Tris-HCl, 150 mM NaCl, pH 7.5 in Water |
| Flow Rate: | 0.30 mL/minute |
| Column: | Waters BioSuite UHR SEC, 125 A, 4 um, 4.6 × |
| | 300 mm |
| Column Temperature: | 30° C. |
| Autosampler | 5° C. |
| Injection Volume: | 10 μL |
| Detector: | UV Detection |
| Detection Wavelength: | 260 nm |
| Run Time | 20 minutes |

TABLE 3
Exemplary Injection Sequence

| Sample/Standard | Number of Injections |
|---|---|
| Stock Column Conditioning | ≥5 |
| WS 3 (40 μg/mL) SRC | 2 |
| WS 3 (40 μg/mL) System Suitability | 5 |
| Blank (NFW) | 3* |
| WS 1 (20 μg/mL) | 1 |
| WS 2 (30 μg/mL) | 1 |
| WS 3 (40 μg/mL) | 1 |
| WS 4 (50 μg/mL) | 1 |
| WS 5 (60 μg/mL) | 1 |
| WCS | 1 |
| Blank (NFW) | 1 |
| WS 3 (40 μg/mL) Bracket | 1 |
| Sample 1-Replicate 1 | |
| Sample 1-Replicate 2 | |
| Sample 1-Replicate 3 | 1 per replicate |
| Sample 2-Replicate 1 | (6 total injections) |
| Sample 2-Replicate 2 | |
| Sample 2-Replicate 3 | |
| WS 3 (40 μg/mL) Bracket | 1 |
| Sample 3-Replicate 1 | |
| Sample 3-Replicate 2 | |
| Sample 3-Replicate 3 | 1 per replicate |
| Sample 4-Replicate 1 | (6 total injections) |
| Sample 4-Replicate 2 | |
| Sample 4-Replicate 3 | |
| WS 3 (40 μg/mL) Bracket | 1 |
| Blank (NFW) | 1 |

*Last injection used for the Non-interference evaluation.

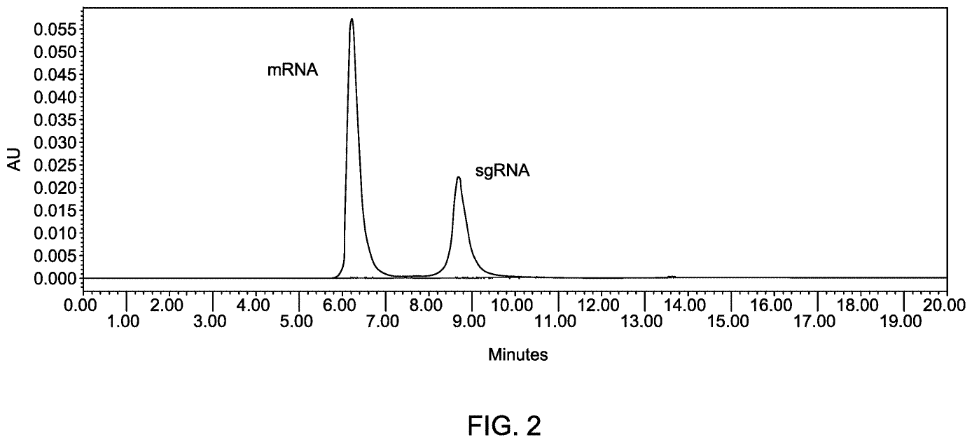

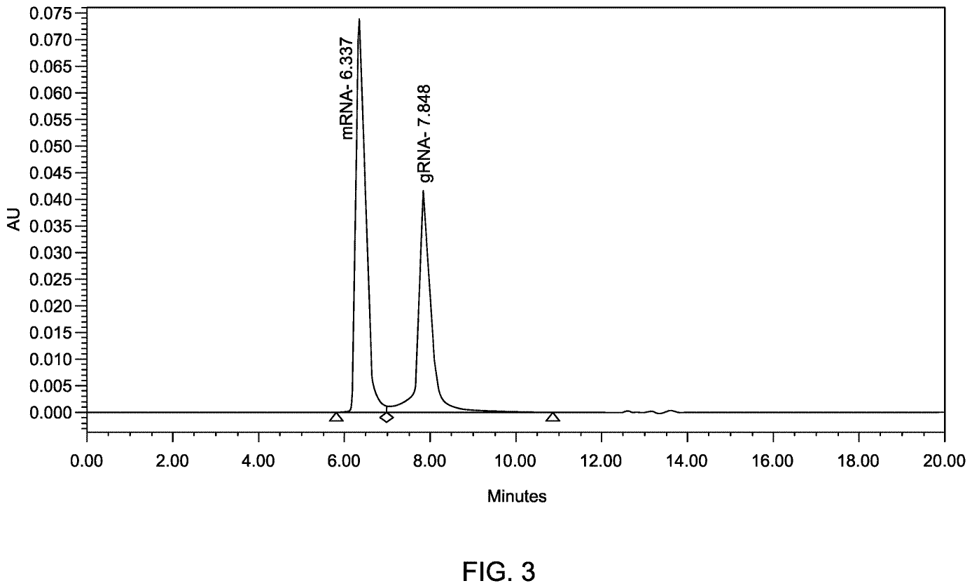

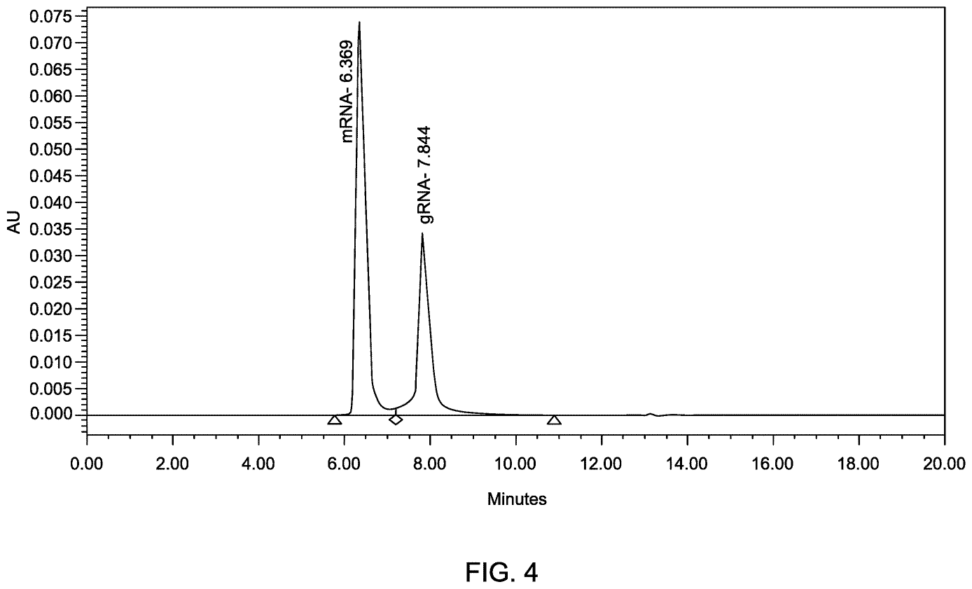

Using the calibration curves, the mRNA and sgRNA concentrations were calculated in μg/mL in samples. The calculated concentration was corrected by the dilution factor, which was 37.5-fold (5-fold initial dilution during extraction×7.5-fold post-extraction dilution). Chromatographic profiles for samples were accepted based on USP Resolution between the mRNA and sgRNA peaks equal to or greater than 1.5, with % RSD for the total RNA concentration in mg/mL across replicates equal to or less than 10%. Exemplary chromatographic profiles are provided in FIGS. 2-4.

Nucleic acid separation according to Examples 1 and 2 were demonstrated to provide various advantages exemplified herein. The present Examples demonstrate that methods of nucleic acid separation provided in the present disclosure provide robust separation of nucleic acids in combination with ease of sample preparation (e.g., fewer and/or less burdensome steps required for nucleic acid extraction as compared to various methods known in the art). High-purity nucleic acid extracts were prepared from complex samples including LNPs, and were highly compatible with chromatography at least in that they were free or substantially free of materials such as lipids, buffers, organic solvents, and/or detergents that are incompatible with chromatographic technique such as SEC (e.g., can cause turbidity and/or be detrimental to chromatography performance). The present Examples demonstrate that methods of nucleic acid separation provided in the present disclosure also provide high accuracy, precision, and recovery, including when applied to small nucleic acids (e.g., sgRNA) and low concentrations of nucleic acid (e.g., total nucleic acid concentration below 100 μg/mL).

### Example 3: Nucleic Acid Extraction

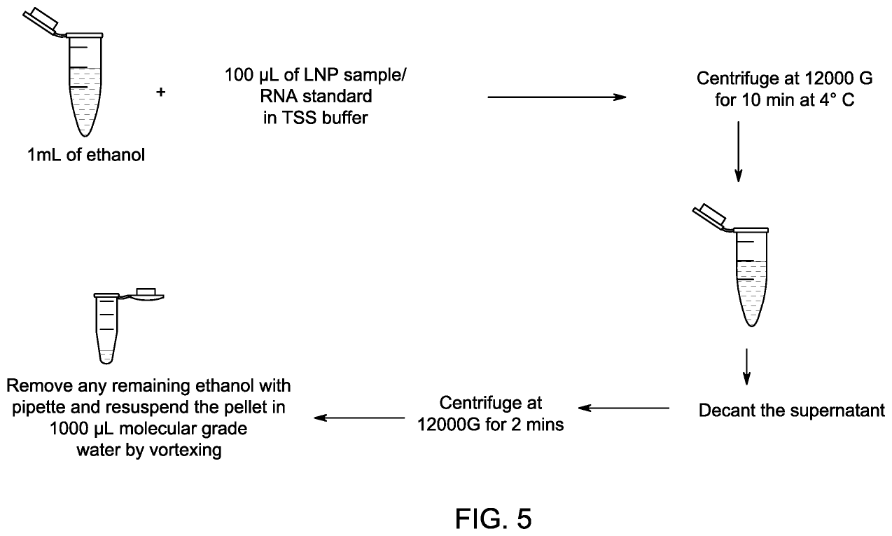

This Example provides exemplary steps for extraction of nucleic acids (here, 2 mRNAs and a sgRNA) from a lipid nucleic acid assembly composition in which the nucleic acids are encapsulated in LNPs. Exemplary steps for extraction of nucleic acids are also provided in FIG. 5.

1) 1000 μL of Ethanol (Absolute Ethanol; 200 Proof) was added to a suitable RNase-Free tube for each lipid nucleic acid assembly composition.

2) 100 μL of LNP sample was added to each ethanol tube and vortexed for 5 seconds.

3) Tubes were centrifuged at 12,000 RCF for 10 minutes at 4° C. After centrifugation, a white RNA pellet was observed at the bottom of the tube.

4) The supernatant ethanol solution was decanted and discarded without disturbing the pellet at the bottom of the tube.

5) Tubes were centrifuged at 12,000 RCF for 2 minutes at 4° C. After centrifugation, a white RNA pellet was observed at the bottom of the tube.

6) Remaining ethanol was removed by pipette.

7) 1000 μL of RNase Free water was added to each tube and vortexed until the pellet was completely dissolved. This solution is a 10× dilution of the cargo.

8) This solution was further diluted so that the final RNA concentration is approximately 50 μg/mL.

The extraction procedure diluted the sample nucleic acid concentration by 10-fold. For example, if the sample was provided at the nominal concentration of 1.5 mg/mL total RNA, the extracted sample should have 150 μg/mL of total RNA, provided 100% recovery during extraction. Dilution can be adjusted to produce a nucleic acid extract having a nucleic acid concentration within the range of a desired calibration curve, and preferably at or near the midpoint of the calibration curve. Nucleic acid extracts were further subjected to denaturation and cooling prior to transfer to HPLC vials for separation.

### Example 4: Ion-Pair Reverse Phase (IP RP) Chromatography of Nucleic Acid Extract

The present Example provides reagents and steps for IP RP chromatography of nucleic acids in a nucleic acid extract prepared according to Example 3, and determination of nucleic acid concentration.

One step of the IP RP chromatography protocol provided herein was preparation of a dilution series for a calibration curve. In the present Examples, the calibration curve range was for the standards having 12-100 μg/mL total RNA concentration. The present example includes a calibration curve in which a stock solution (Table 4) was used to prepare a series of five dilutions, each dilution including the reference 0.1 kb sgRNA, the reference 1.0 kb mRNA, and the reference 4.5 kb mRNA with a respective concentration ratio of 1:2:1, and a total RNA concentration as shown in Table 5.

TABLE 4
Exemplary Standard RNA Test Packet

| | Volume, 100 |
|---|---|
| | μg/mL Stock |
| | Solution |
| 0.1 kb sgRNA | 200 μL |
| 1.0 kb mRNA | 400 μL |
| 4.5 kb mRNA | 200 μL |

TABLE 5
Exemplary Dilution Series for Calibration Curve

| | Total RNA |
|---|---|
| Points | Concentration |
| 1 | 100 μg/mL |
| 2 | 75 μg/mL |
| 3 | 50 μg/mL |
| 4 | 25 μg/mL |
| 5 | 12 μg/mL |

One step of the IP RP chromatography protocol provided herein was preparation of the chromatography mobile phase. The IP RP chromatography mobile phase was prepared as 50 mM Dibutylammonium acetate, 100 mM Triethylammonium acetate in water, or 50 mM Dibutylammonium acetate, 100 mM Triethylammonium acetate, 50% acetonitrile in water.

The IP RP chromatography was performed using a DNAPac™ RP Column, 4 μm, 2.1×100 mm column (Catalog #DX088923) and an HPLC system with UV Detection (260 nm) or equivalent equipped with: autosampler (capable to maintain 4±2° C.), column heater (capable to maintain 65° C.) and capable to deliver 10 μL injection volume. Standard methodologies were applied for column preparation including equilibration and conditioning. Operating conditions and an exemplary injection sequence are provided below, showing triplicate testing of each sample.

TABLE 6
Exemplary Operating Conditions

| | |
|---|---|
| Mobile Phase: | 50 mM Dibutylammonium acetate, 100 mM |
| | Triethylammonium acetate in water |
| Flow Rate: | 0.45 mL/minute |
| Column: | DNAPac ™ RP Column, 4 μm, 2.1 × |
| | 100 mm |
| Column Temperature: | 65° C. |
| Autosampler Temperature: | 4° C. |
| Injection Volume: | 10 μL |
| Detector: | UV Detection |
| Detection Wavelength: | 260 nm |
| Run Time | 20 minutes |

TABLE 7
Exemplary Operating Conditions

| | |
|---|---|
| Mobile Phase: | 50 mM Dibutylammonium acetate, 100 mM |
| | Triethylammonium acetate, 50% acetonitrile |
| | in water |
| Flow Rate: | 0.45 mL/minute |
| Column: | DNAPac ™ RP Column, 4 μm, 2.1 × 100 |
| | mm |
| Column Temperature: | 65° C. |
| Autosampler Temperature: | 4° C. |
| Injection Volume: | 10 μL |
| Detector: | UV Detection |
| Detection Wavelength: | 260 nm |
| Run Time | 20 minutes |

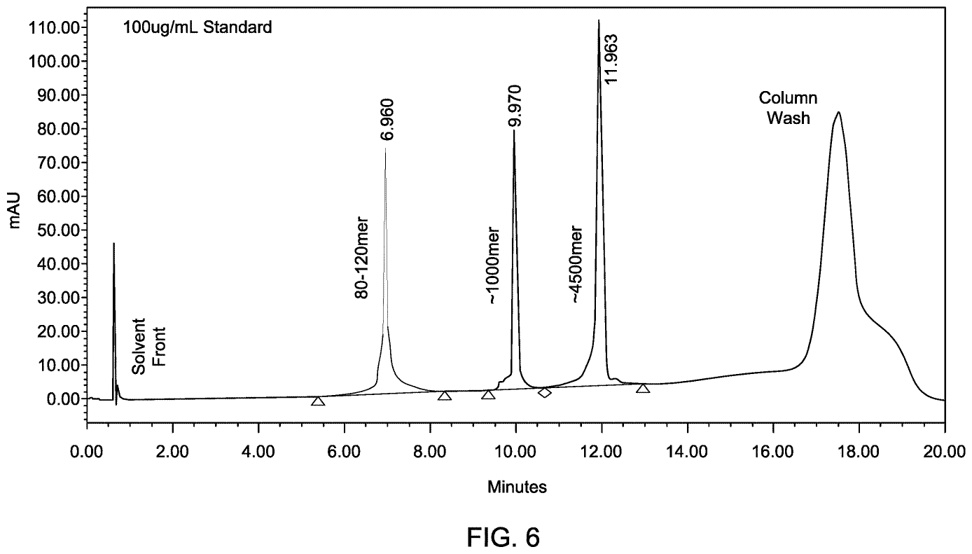

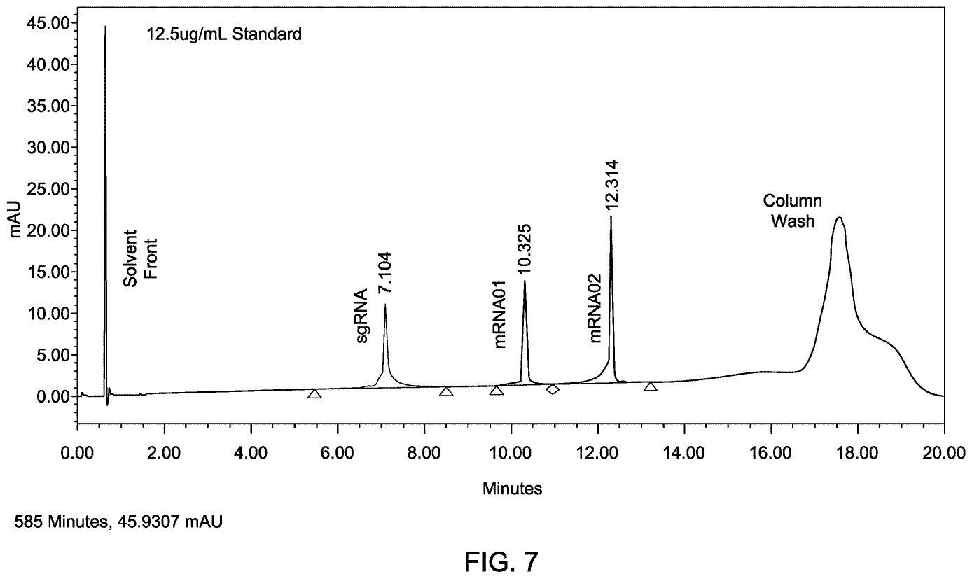

Using the calibration curves, the mRNA and sgRNA concentrations were calculated in μg/mL in samples. The calculated concentrations were corrected by the cargo dilution factor, which was 30-fold (10-fold initial dilution during extraction×3-fold post-extraction dilution). Chromatographic profiles for samples were accepted based on USP Resolution between the mRNA and sgRNA peaks equal to or greater than 1.5, with % RSD for the total RNA concentration in mg/mL across replicates equal to or less than 10%. Exemplary chromatographic profiles are provided in FIGS. 6-8.

TABLE 8
Exemplary Standard Weigh Out Calculations

| | 100 | 75 | 50 | 25 | 12.5 |
|---|---|---|---|---|---|
| | μg/mL | μg/mL | μg/mL | μg/mL | μg/mL |
| 0.1 kb sgRNA | 25 | 18.75 | 12.5 | 6.25 | 3.125 |
| 1.0 kb mRNA | 50 | 37.5 | 25 | 12.5 | 6.25 |
| 4.5 kb mRNA | 25 | 18.75 | 12.5 | 6.25 | 3.125 |

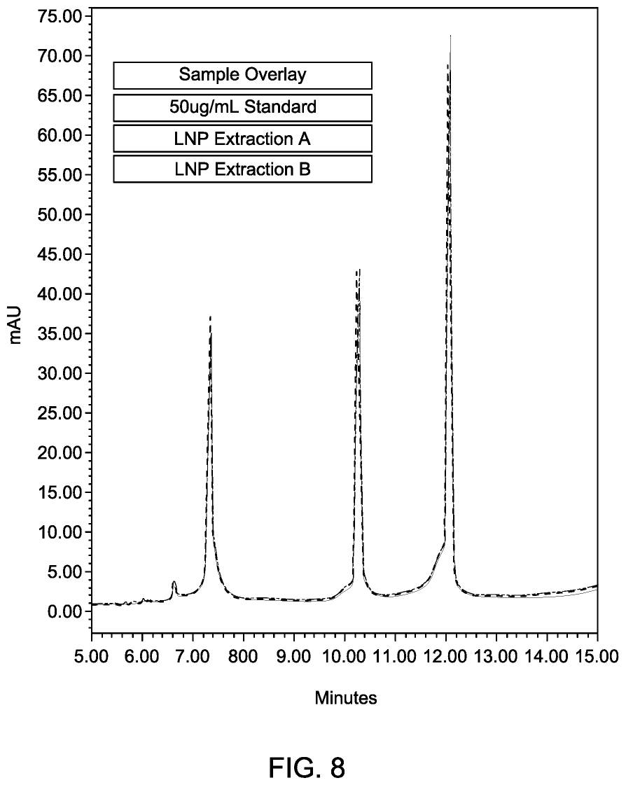

TABLE 9
Exemplary Weigh Out Calculations for the LNP Extractions (FIG. 8)

| | 0.1 kb | 1.0 kb | 4.5 kb | Total | Ratio | | |
|---|---|---|---|---|---|---|---|
| | sgRNA | mRNA | mRNA | Conc. | 0.1 kb | 1.0 kb | 4.5 kb |
| Sample ID | (mg/mL) | (mg/mL) | (mg/mL) | (mg/mL) | sgRNA | mRNA | mRNA |
| LNP Extraction A | 0.38 | 0.72 | 0.39 | 1.48 | 1.0 | 1.9 | 1.1 |
| LNP Extraction B | 0.39 | 0.73 | 0.38 | 1.49 | 1.0 | 2.0 | 1.0 |
| % Difference | −2.2% | −0.8% | 2.0% | −0.4% | −1.9% | −0.7% | 3.3% |
| Avg | 0.38 | 0.72 | 0.38 | 1.49 | 1.0 | 2.0 | 1.0 |
| Target* | 0.37 | 0.74 | 0.37 | 1.49 | 1.0 | 2.0 | 1.0 |
| % Difference from | 2.7% | −2.7% | 2.7% | 0.0% | 0.0% | 0.0% | 0.0% |
| Target | | | | | | | |

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. For example, elements of claims that depend directly or indirectly from a certain independent claim presented herein serve as support for those elements being presented in additional dependent claims of one or more other independent claims. Throughout the description, where compositions or methods are described as having, including, or comprising specific elements, compositions that consist essentially of, consist of, or do not comprise the recited elements are likewise hereby disclosed. All references cited herein are hereby incorporated by reference.

What is claimed is:

1. A method comprising:
a) preparing a nucleic acid extract from the lipid nucleic acid assembly composition, wherein the preparation of the nucleic acid extract comprises ethanol precipitation of the nucleic acid from the lipid nucleic acid assembly composition; and
b) subjecting the nucleic acid extract to size exclusion chromatography (SEC).

2. The method of claim 1, wherein the method further comprises determining the total concentration of the nucleic acid present in the lipid nucleic acid assembly composition.

3. A method of determining the total concentration of nucleic acid present in a lipid nucleic acid assembly composition, the method comprising:
a) preparing a nucleic acid extract from the lipid nucleic acid assembly composition, wherein the preparation of the nucleic acid extract comprises ethanol precipitation of the nucleic acid from the lipid nucleic acid assembly composition;
b) subjecting the nucleic acid extract to size exclusion chromatography (SEC); and
c) determining the total concentration of the nucleic acid in the lipid nucleic acid assembly composition.

4. The method of any one of claims 1-3, wherein the nucleic acid present in the lipid nucleic acid assembly composition comprises at least a first nucleic acid and a second nucleic acid, and wherein the SEC separates the first nucleic acid from the second nucleic acid.

5. The method of claim 4, wherein the method further comprises determining the concentration of the first nucleic acid and/or the second nucleic acid in the lipid nucleic acid assembly composition.

6. The method of any one of claims 1-3, wherein the nucleic acid present in the lipid nucleic acid assembly composition comprises at least a first nucleic acid, a second nucleic acid, and a third nucleic acid, and wherein the SEC separates the first nucleic acid from the second nucleic acid and/or the third nucleic acid.

7. The method of any one of claims 4-6, wherein the first nucleic acid is a ribonucleic acid (RNA) molecule.

8. The method of claim 7, wherein the first nucleic acid is a messenger RNA (mRNA) molecule.

9. The method of claim 8, wherein the mRNA molecule encodes a polypeptide, optionally wherein the polypeptide comprises an RNA-guided DNA binding agent, optionally wherein the RNA-guided DNA binding agent comprises a nuclease, optionally wherein the nuclease is a Cas protein, optionally wherein the RNA-guided DNA binding agent is a fusion polypeptide.

10. The method of claim 8 or 9, wherein the first nucleic acid encodes a base editor.

11. The method of claim 10, wherein the base editor is a cytosine base editor.

12. The method of claim 10, wherein the base editor is an adenine base editor.

13. The method of any one of claims 4-12, wherein the first nucleic acid comprises 1000-7000 nucleotides.

14. The method of any of one of claims 4-13, wherein the second nucleic acid is an RNA molecule.

15. The method of claim 14, wherein the second nucleic acid is a guide RNA (gRNA) molecule, optionally wherein the gRNA molecule is an sgRNA molecule.

16. The method of claim 14 or 15, wherein the second nucleic acid comprises 75-200 nucleotides, optionally wherein the second nucleic acid comprises 80-120 nucleotides.

17. The method of any one of claims 4-16, wherein the nucleic acid present in the lipid nucleic acid assembly composition consists or consists essentially of the first nucleic acid and the second nucleic acid.

18. The method of any one of claims 4-17, wherein the total concentration of nucleic acid in the lipid nucleic acid assembly composition is the sum of the concentration of the first nucleic acid and the concentration of the second nucleic acid.

19. The method of any one of claims 4-18, wherein the concentration of the first nucleic acid and/or the second nucleic acid is determined according to a calibration curve, wherein the calibration curve represents the concentration of nucleic acid in a dilution series of at least one reference nucleic acid.

20. The method of claim 19, wherein the concentration of the first nucleic acid and the second nucleic acid are each determined, wherein the concentration of the first nucleic acid is determined according to a first calibration curve and the concentration of the second nucleic acid is determined according to a second calibration curve.

21. The method of claim 20, wherein the first nucleic acid is an mRNA molecule and the first calibration curve is a dilution series of a reference mRNA molecule.

22. The method of claim 20 or 21, wherein the second nucleic acid is a gRNA molecule and the second calibration curve is a dilution series of a reference gRNA molecule.

23. The method of any one of claims 20-22, wherein the first calibration curve represents a first dilution series including concentrations of a reference mRNA molecule that encompass a range including 12 μg/mL and 60 μg/mL, and/or a range including 12 μg/mL and 36 μg/mL.

24. The method of any one of claims 20-23, wherein the second calibration curve represents a second dilution series including concentrations of a reference gRNA molecule that encompasses a range including 8 μg/mL and 40 μg/mL, and/or a range including 8 μg/mL and 24 μg/mL.

25. The method of any one of claims 20-24 wherein the first dilution series and second dilution series include concentrations such that pairwise addition of a concentration from the first dilution series and a concentration from the second dilution series yields total RNA concentrations that encompass a range including 20 μg/mL and 100 μg/mL, or a range including 20 μg/mL and 60 μg/mL

26. The method of any one of claims 4-25, wherein the method comprises determining a concentration of the first nucleic acid and/or the second nucleic acid, and/or a total nucleic acid concentration, of the lipid nucleic acid assembly composition, that is equal to or less than 2.5 mg/mL, 2.0 mg/mL, 1.5 mg/mL, 1.0 mg/mL, 0.5 mg/mL, 0.4 mg/mL, or 0.3 mg/mL.

27. The method of any one of claims 4-26, wherein the method comprises determining a concentration of the first nucleic acid and/or the second nucleic acid, and/or a total nucleic acid concentration, where the total nucleic acid concentration is between about 0.5 mg/mL and about 2 mg/mL, optionally wherein the total nucleic acid concentration is between about 0.75 mg/mL and about 1.8 mg/mL.

28. The method of any one of claims 4-27, wherein the SEC produces a chromatographic profile in which USP resolution between a nucleic acid peak representing the first nucleic acid and a nucleic acid peak representing the second nucleic acid is equal to or greater than 1.5.

29. The method of any one of claims 1-28, wherein the preparation of the nucleic acid extract comprises diluting the concentration of the nucleic acid extract to a midpoint of a calibration curve.

30. The method of any one of claims 1-29, wherein the lipid nucleic acid assembly composition comprises lipid nanoparticles (LNPs) comprising the nucleic acid.

31. The method of claim 30, wherein the lipid component of the LNPs comprises (i) an amine lipid, (ii) a helper lipid, and (iii) a stealth lipid, optionally wherein the LNPs comprise a neutral lipid, optionally wherein the stealth lipid is a PEG lipid.

32. The method of any one of claims 4-31, wherein:
the first nucleic acid is a messenger RNA (mRNA) molecule encoding an RNA-guided DNA binding agent,
the second nucleic acid is a gRNA molecule,
the lipid nucleic acid assembly composition comprises LNPs comprising molecules of the first nucleic acid and the second nucleic acid, and
the lipid component of the LNPs comprises (i) an amine lipid, (ii) a helper lipid, (iii) a stealth lipid, and (iv) a neutral lipid, optionally wherein the stealth lipid is a PEG lipid.

33. The method of any one of claims 1-32, wherein the total concentration of nucleic acid in the lipid nucleic acid assembly composition is the total concentration of RNA in the lipid nucleic acid assembly composition.

34. The method of any one of claims 1-33, wherein the ethanol precipitation comprises:
a) mixing the lipid nucleic acid assembly composition with ethanol to produce an ethanol mixture;
b) centrifuging the ethanol mixture, wherein the centrifugation produces a nucleic acid pellet and an ethanol supernatant; and
c) isolating the nucleic acid pellet from the ethanol, wherein the nucleic acid pellet comprises precipitated RNA.

35. The method of claim 34, wherein the isolating of the pellet comprises decanting supernatant and/or drying the nucleic acid pellet.

36. The method of claim 34 or 35, wherein the preparation of the nucleic acid extract comprises resuspending the nucleic acid pellet in water to produce an aqueous nucleic acid solution.

37. The method of any one of claims 34-36, wherein preparation of the nucleic acid extract comprises heating the aqueous nucleic acid solution to a temperature between about 65° C. and about 90° C. for at least about 2 minutes to about 15 minutes, optionally wherein the preparation of the nucleic acid extract comprises, after the heating, cooling the aqueous nucleic solution, optionally wherein the cooling is to 4° C.

38. The method of any one of claims 1-37, wherein the nucleic acid extract is free or substantially free of lipid, free or substantially free of buffer, and/or free or substantially free of detergents.

39. The method of any one of claims 1-38, wherein the SEC is a High Performance Liquid Chromatography (HPLC).

40. The method of any one of claims 1-39, wherein subjecting the nucleic acid extract to SEC comprises contacting the nucleic acid extract with an SEC substrate, optionally wherein the SEC substrate is, or is present in, an SEC column.

41. The method of claim 40, wherein subjecting the nucleic acid extract to SEC comprises contacting the nucleic acid extract with an SEC column having internal diameter of 3 mm to 6 mm, optionally having an internal diameter of about 4 mm to about 5 mm, optionally having an internal diameter of about 4.6 mm internal diameter.

42. The method of claim 40 or 41, wherein subjecting the nucleic acid extract to SEC comprises contacting the nucleic acid extract with an SEC column having a length of about 100 mm to about 400 mm, optionally having a length of about 300 mm length.

43. The method of any one of claims 1-42, wherein subjecting the nucleic acid extract to SEC comprises contacting the nucleic acid extract with an SEC substrate and/or SEC column having a pore size of about 5 nm to about 50 nm, optionally wherein the pore size is about 12.5 nm to about 25 nm.

44. The method of any one of claims 1-43, wherein subjecting the nucleic acid extract to SEC comprises contacting the nucleic acid extract with an SEC substrate and/or SEC column having a particle size of about 3 μm to about 5 μm, optionally a particle size of about 4 μm.

45. The method of any one of claims 1-44, wherein subjecting the nucleic acid extract to SEC comprises contacting the nucleic acid extract with an SEC substrate and/or SEC column comprising silica, optionally wherein the SEC substrate and/or SEC column comprises silica particles.

46. The method of any one of claims 1-45, wherein subjecting the nucleic acid extract to SEC comprises contacting the nucleic acid extract with an SEC substrate and/or SEC column compatible with a mobile phase having a pH of about 7 to about 8, optionally wherein the SEC substrate and/or SEC column is compatible with a mobile phase having a pH of about 7.3 to about 7.7, optionally wherein the SEC substrate and/or SEC column is compatible with a mobile phase having a pH of about 7.5.

47. The method of any one of claims 1-46, wherein subjecting the nucleic acid extract to SEC comprises contacting the nucleic acid extract with an SEC substrate and/or SEC column compatible with a mobile phase comprising Tris-HCl, optionally wherein the SEC substrate and/or SEC column is compatible with a mobile phase comprising about 5 mM to about 50 mM Tris-HCl, optionally wherein the SEC substrate and/or SEC column is compatible with a mobile phase comprising about 20 mM Tris-HCl.

48. The method of any one of claims 1-47, wherein subjecting the nucleic acid extract to SEC comprises contacting the nucleic acid extract with an SEC substrate and/or SEC column compatible with a mobile phase comprising about 25 mM to about 200 mM NaCl, optionally wherein the SEC substrate and/or SEC column is compatible with a mobile phase comprising about 150 mM NaCl.

49. The method of any one of claims 1-48, wherein subjecting the nucleic acid extract to SEC comprises contacting the nucleic acid extract with an SEC mobile phase having a pH between about 7 and about 8, optionally wherein the mobile phase has a pH of about 7.3 to about 7.7, optionally wherein the mobile phase has a pH of about 7.5.

50. The method of any one of claims 1-49, wherein subjecting the nucleic acid extract to SEC comprises contacting the nucleic acid extract with an SEC mobile phase comprising Tris-HCl, optionally wherein the SEC mobile phase comprises about 5 mM to about 50 mM Tris-HCl, optionally wherein the SEC mobile phase comprises about 20 mM Tris-HCl.

51. The method of any one of claims 1-50, wherein subjecting the nucleic acid extract to SEC comprises contacting the nucleic acid extract with an SEC mobile phase comprising NaCl, optionally wherein the SEC mobile phase comprises about 25 mM to about 200 mM NaCl, optionally wherein the SEC mobile phase comprises about 150 mM NaCl.

52. The method of any one of claims 1-51, wherein the method comprises measuring absorbance of SEC eluate at 260 nm.

53. The method of any one of claims 1-52, wherein the SEC is carried out at a temperature of 20-40° C., optionally wherein the SEC is carried out at a temperature of 30° C.

54. A kit for separating nucleic acid present in a lipid nucleic acid assembly composition, the kit comprising a size exclusion chromatography (SEC) substrate and at least one reagent for ethanol precipitation of the nucleic acid from the lipid nucleic acid assembly composition, optionally wherein the kit is for separating two or more nucleic acids, optionally wherein the two or more nucleic acid molecules are or include a gRNA and an mRNA.

55. The kit of claim 54, wherein the SEC substrate is, or is present in, an SEC column.

56. The kit of claim 55, wherein the SEC column has an internal diameter of 3 mm to 6 mm, optionally an internal diameter of about 4 mm to about 5 mm, optionally an internal diameter of about 4.6 mm internal diameter.

57. The kit of claim 55 or 56, wherein the SEC column has a length of about 100 mm to about 400 mm, optionally a length of about 300 mm length.

58. The kit of any one of claims 54-57, wherein the SEC substrate and/or SEC column has a pore size of about 5 nm to about 50 nm, optionally wherein the pore size is about 12.5 nm to about 25 nm.

59. The kit of any one of claims 54-58, wherein the SEC substrate and/or SEC column has a particle size of about 3 μm to about 5 μm, optionally a particle size of about 4 μm.

60. The method of any one of claims 54-59, wherein the SEC substrate and/or SEC column comprises silica, optionally wherein the SEC substrate comprises silica particles.

61. The kit of any one of claims 54-50, wherein the SEC substrate and/or SEC column is compatible with a mobile phase having a pH of about 7 to about 8, optionally a mobile phase having a pH of about 7.3 to about 7.7, optionally a mobile phase having a pH of about 7.5.

62. The kit of any one of claims 54-61, wherein the SEC substrate and/or SEC column is compatible with a mobile phase comprising about 25 mM to about 200 mM NaCl, optionally a mobile phase comprising about 150 mM NaCl.

63. The kit of any one of claims 54-62, wherein the kit comprises a dilution series of one or more reference nucleic acids, optionally wherein the kit comprises a dilution series of a reference gRNA molecule and/or a reference mRNA molecule.

64. The kit of any one of claims 54-63, wherein the kit comprises instructions for preparation of a nucleic acid extract by a process comprising ethanol precipitation.

65. The kit of any one of claims 54-64, wherein the kit comprises instructions for separating the two or more nucleic acids by SEC, optionally wherein the two or more nucleic acids comprise or consist of RNA molecules, optionally wherein the RNA molecules comprise or consist of a gRNA molecule and an mRNA molecule.

66. The kit of any one of claims 54-65, wherein the kit comprises instructions for determining the concentration of the two more nucleic acids and/or total nucleic acid concentration, optionally wherein the two or more nucleic acids comprise or consist of RNA molecules, optionally wherein the RNA molecules comprise or consist of a gRNA molecule and an mRNA molecule.

67. A method comprising:
a) preparing a nucleic acid extract from the lipid nucleic acid assembly composition, wherein the preparation of the nucleic acid extract comprises ethanol precipitation of the nucleic acid from the lipid nucleic acid assembly composition; and
b) subjecting the nucleic acid extract to ion pair chromatography.

68. The method of claim 67, wherein the method further comprises determining the total concentration of the nucleic acid present in the lipid nucleic acid assembly composition.

69. A method of determining the total concentration of nucleic acid present in a lipid nucleic acid assembly composition, the method comprising:
a) preparing a nucleic acid extract from the lipid nucleic acid assembly composition, wherein the preparation of the nucleic acid extract comprises ethanol precipitation of the nucleic acid from the lipid nucleic acid assembly composition;
b) subjecting the nucleic acid extract to ion pair chromatography; and
c) determining the total concentration of the nucleic acid in the lipid nucleic acid assembly composition.

70. The method of any one of claims 67-69, wherein the nucleic acid present in the lipid nucleic acid assembly composition comprises at least a first nucleic acid and a second nucleic acid, and wherein the ion pair chromatography separates the first nucleic acid from the second nucleic acid.

71. The method of claim 70, wherein the method further comprises determining the concentration of the first nucleic acid and/or the second nucleic acid in the lipid nucleic acid assembly composition.

72. The method of any one of claims 67-69, wherein the nucleic acid present in the lipid nucleic acid assembly composition comprises at least a first nucleic acid, a second nucleic acid, and a third nucleic acid, and wherein the SEC separates the first nucleic acid from the second nucleic acid and/or the third nucleic acid.

73. The method of any one of claims 70-72, wherein the first nucleic acid is a ribonucleic acid (RNA) molecule.

74. The method of claim 73, wherein the first nucleic acid is a messenger RNA (mRNA) molecule.

75. The method of claim 74, wherein the mRNA molecule encodes a polypeptide, optionally wherein the polypeptide comprises an RNA-guided DNA binding agent, optionally wherein the RNA-guided DNA binding agent comprises a nuclease, optionally wherein the nuclease is a Cas protein, optionally wherein the RNA-guided DNA binding agent is a fusion polypeptide.

76. The method of claim 74 or 75, wherein the first nucleic acid encodes a base editor.

77. The method of claim 76, wherein the base editor is a cytosine base editor.

78. The method of any one of claims 70-77, wherein the first nucleic acid comprises 1000-7000 nucleotides.

79. The method of any of one of claims 70-78, wherein the second nucleic acid is an RNA molecule.

80. The method of claim 79, wherein the second nucleic acid is a guide RNA (gRNA) molecule, optionally wherein the gRNA molecule is an sgRNA molecule.

81. The method of claim 79 or 80, wherein the second nucleic acid comprises 75-200 nucleotides, optionally wherein the second nucleic acid comprises 80-120 nucleotides.

82. The method of any one of claims 70-81, wherein the third nucleic acid is a ribonucleic acid (RNA) molecule.

83. The method of claim 82, wherein the third nucleic acid is a messenger RNA (mRNA) molecule.

84. The method of claim 83, wherein the mRNA molecule encodes a polypeptide, optionally wherein the polypeptide is a DNA glycosylase inhibitor, optionally wherein the DNA glycosylase inhibitor is UGI.

85. The method of any one of claims 70-84, wherein the third nucleic acid comprises 750-3000 nucleotides.

86. The method of any one of claims 70-85, wherein the nucleic acid present in the lipid nucleic acid assembly composition consists or consists essentially of (i) the first nucleic acid and the second nucleic acid, or (ii) the first nucleic acid, the second nucleic acid, and the third nucleic acid.

87. The method of any one of claims 70-86, wherein the total concentration of nucleic acid in the lipid nucleic acid assembly composition is the sum of (i) the concentration of the first nucleic acid and the concentration of the second nucleic acid, or (ii) the concentration of the first nucleic acid, the concentration of the second nucleic acid, and the concentration of the third nucleic acid.

88. The method of any one of claims 70-87, wherein (i) the concentration of the first nucleic acid and/or the second nucleic acid is determined according to a calibration curve, or (ii) the concentration of the first nucleic acid, the second nucleic acid, and/or the third nucleic acid is determined according to a calibration curve, wherein the calibration curve represents the concentration of nucleic acid in a dilution series of at least one reference nucleic acid.

89. The method of claim 88, wherein (i) the concentration of the first nucleic acid and the second nucleic acid are each determined and/or (ii) the concentration of the first nucleic acid, the second nucleic acid, and the third nucleic acid are each determined, wherein the concentration of the first nucleic acid is determined according to a first calibration curve, the concentration of the second nucleic acid is determined according to a second calibration curve, and the concentration of the third nucleic acid, when present, is determined according to a third calibration curve.

90. The method of claim 89, wherein the first nucleic acid is an mRNA molecule and the first calibration curve is a dilution series of a first reference mRNA molecule, optionally wherein the length and/or sequence of the first reference mRNA molecule is the same as the first nucleic acid or differs from the first nucleic acid by no more than 10%, no more than 5%, or no more than 1%.

91. The method of claim 89 or 90 wherein the second nucleic acid is a gRNA molecule and the second calibration curve is a dilution series of a reference gRNA molecule, optionally wherein the length and/or sequence of the reference gRNA molecule is the same as the second nucleic acid or differs from the second nucleic acid by no more than 10%, no more than 5%, or no more than 1%.

92. The method of any one of claims 89-91, wherein the third nucleic acid is an mRNA molecule and the third calibration curve is a dilution series of a second reference mRNA molecule, optionally wherein the length and/or sequence of the second reference mRNA molecule is the same as the third nucleic acid or differs from the third nucleic acid by no more than 10%, no more than 5%, or no more than 1%.

93. The method of any one of claims 89-92, wherein the first calibration curve represents a first dilution series including concentrations of a first reference mRNA molecule that encompass a range including 10 μg/mL and 100 μg/mL.

94. The method of any one of claims 89-93, wherein the second calibration curve represents a second dilution series including concentrations of a reference gRNA molecule that encompasses a range including 5 μg/mL and 100 μg/mL, and/or a range including 5 μg/mL and 50 μg/mL.

95. The method of any one of claims 89-94, wherein the third calibration curve represents a third dilution series including concentrations of a second reference mRNA molecule that encompass a range including 10 μg/mL and 100 μg/mL.

96. The method of any one of claims 89-95, wherein the first dilution series and second dilution series include concentrations such that total concentration from the first dilution series, second dilution series, and third dilution series yields total RNA concentrations that encompass a range including 20 μg/mL and 200 μg/mL, or a range including 20 μg/mL and 100 μg/mL

97. The method of any one of claims 70-96, wherein the method comprises determining a concentration of the first nucleic acid, the second nucleic acid, and/or the third nucleic acid, and/or a total nucleic acid concentration, of the lipid nucleic acid assembly composition, that is equal to or less than 2.5 mg/mL, 2.0 mg/mL, 1.5 mg/mL, 1.0 mg/mL, 0.5 mg/mL, 0.4 mg/mL, or 0.3 mg/mL.

98. The method of any one of claims 70-97, wherein the method comprises determining a concentration of the first nucleic acid, the second nucleic acid, and/or the third nucleic acid, and/or a total nucleic acid concentration, where the total nucleic acid concentration is between about 0.5 mg/mL and about 2 mg/mL, optionally wherein the total nucleic acid concentration is between about 0.75 mg/mL and about 1.8 mg/mL.

99. The method of any one of claims 70-98, wherein the ion pair chromatography produces a chromatographic profile in which USP resolution between a nucleic acid peak representing the first nucleic acid and a nucleic acid peak representing the second nucleic acid is equal to or greater than 1, 1.5, 2, 2.5, or 3.

100. The method of any one of claims 70-99, wherein the ion pair chromatography produces a chromatographic profile in which USP resolution between a nucleic acid peak representing the first nucleic acid and a nucleic acid peak representing the third nucleic acid is equal to or greater than 1, 1.5, 2.0, 2.5, 3.0, 3.5, 4.0, 4.5, or 5.

101. The method of any one of claims 70-100, wherein the ion pair chromatography produces a chromatographic profile in which USP resolution between a nucleic acid peak representing the second nucleic acid and a nucleic acid peak representing the third nucleic acid is equal to or greater than 1, 1.5, or 2.

102. The method of any one of claims 67-101, wherein the preparation of the nucleic acid extract comprises diluting the concentration of the nucleic acid extract to a midpoint of a calibration curve.

103. The method of any one of claims 67-102, wherein the lipid nucleic acid assembly composition comprises lipid nanoparticles (LNPs) comprising the nucleic acid.

104. The method of claim 103, wherein the lipid component of the LNPs comprises (i) an amine lipid, (ii) a helper lipid, and (iii) a stealth lipid, optionally wherein the LNPs comprise a neutral lipid, optionally wherein the stealth lipid is a PEG lipid.

105. The method of any one of claims 72-104, wherein:
the first nucleic acid is a messenger RNA (mRNA) molecule encoding a cytosine base editor,
the second nucleic acid is a gRNA molecule, optionally wherein the gRNA molecule is an sgRNA molecule, and
the third nucleic acid is an mRNA molecule encoding a DNA glycosylase inhibitor, optionally wherein the DNA glycosylase inhibitor is UGI.

106. The method of claim 105, wherein the first nucleic acid does not encode a DNA glycosylase inhibitor and/or wherein the first nucleic acid does not encode UGI.

107. The method of claim 105, wherein the cytosine base editor comprises a DNA glycosylase inhibitor, optionally wherein the DNA glycosylase inhibitor is UGI.

108. The method of any one of claims 72-107, wherein:
the first nucleic acid is a messenger RNA (mRNA) molecule encoding a cytosine base editor,
the second nucleic acid is a gRNA molecule,
the third nucleic acid is an mRNA molecule encoding a DNA glycosylase inhibitor, optionally wherein the DNA glycosylase inhibitor is uracil glycosylase inhibitor (UGI),
the lipid nucleic acid assembly composition comprises LNPs comprising molecules of the first nucleic acid and the second nucleic acid, and
the lipid component of the LNPs comprises (i) an amine lipid, (ii) a helper lipid, (iii) a stealth lipid, and (iv) a neutral lipid, optionally wherein the stealth lipid is a PEG lipid.

109. The method of any one of claims 67-108, wherein the total concentration of nucleic acid in the lipid nucleic acid assembly composition is the total concentration of RNA in the lipid nucleic acid assembly composition.

110. The method of any one of claims 67-109, wherein the ethanol precipitation comprises:
a) mixing the lipid nucleic acid assembly composition with ethanol to produce an ethanol mixture;
b) centrifuging the ethanol mixture, wherein the centrifugation produces a nucleic acid pellet and an ethanol supernatant; and
c) isolating the nucleic acid pellet from the ethanol, wherein the nucleic acid pellet comprises precipitated RNA.

111. The method of claim 110, wherein the isolating of the pellet comprises decanting supernatant, pipetting away supernatant, and/or drying the nucleic acid pellet.

112. The method of claim 110 or 111, wherein the preparation of the nucleic acid extract comprises resuspending the nucleic acid pellet in water to produce an aqueous nucleic acid solution.

113. The method of any one of claims 110-112, wherein preparation of the nucleic acid extract comprises heating the aqueous nucleic acid solution to a temperature between about 65° C. and about 90° C. for at least about 2 minutes to about 15 minutes, optionally wherein the preparation of the nucleic acid extract comprises, after the heating, cooling the aqueous nucleic solution, optionally wherein the cooling is to 4° C.

114. The method of any one of claims 67-113, wherein the nucleic acid extract is free or substantially free of lipid, free or substantially free of buffer, and/or free or substantially free of detergents.

115. The method of any one of claims 67-114, wherein the ion pair chromatography is a reverse phase chromatography.

116. The method of any one of claims 67-115, wherein the ion pair chromatography is a High Performance Liquid Chromatography (HPLC).

117. The method of any one of claims 67-116, wherein the ion pair chromatography is a reverse phase High Performance Liquid Chromatography (HPLC).

118. The method of any one of claims 67-117, wherein subjecting the nucleic acid extract to ion pair chromatography comprises contacting the nucleic acid extract with an ion pair chromatography substrate, optionally wherein the ion pair chromatography substrate is, or is present in, an ion pair chromatography column.

119. The method of claim 118, wherein subjecting the nucleic acid extract to ion pair chromatography comprises contacting the nucleic acid extract with an ion pair chromatography column having internal diameter of 1 mm to 4 mm, optionally having an internal diameter of about 1.5 mm to about 2.5 mm, optionally having an internal diameter of about 2.1 mm internal diameter.

120. The method of claim 118 or 119, wherein subjecting the nucleic acid extract to ion pair chromatography comprises contacting the nucleic acid extract with an ion pair chromatography column having a length of about 50 mm to about 400 mm, optionally having a length of about 100 mm length.

121. The method of any one of claims 67-120, wherein subjecting the nucleic acid extract to ion pair chromatography comprises contacting the nucleic acid extract with an ion pair chromatography substrate and/or ion pair chromatography column having a pore size of about 100 Å to about 3000 Å, optionally wherein the pore size is about 1000 Å to about 2000 Å.

122. The method of any one of claims 67-121, wherein subjecting the nucleic acid extract to ion pair chromatography comprises contacting the nucleic acid extract with an ion pair chromatography substrate and/or ion pair chromatography column having a particle size of about 3 μm to about 5 μm, optionally a particle size of about 4 μm.

123. The method of any one of claims 67-122, wherein subjecting the nucleic acid extract to ion pair chromatography comprises contacting the nucleic acid extract with an ion pair chromatography substrate and/or ion pair chromatography column comprising silica, optionally wherein the ion pair chromatography substrate and/or ion pair chromatography column comprises silica particles.

124. The method of any one of claims 67-123, wherein subjecting the nucleic acid extract to ion pair chromatography comprises contacting the nucleic acid extract with an ion pair chromatography substrate and/or ion pair chromatography column compatible with at least one mobile phase comprising dibutylammonium acetate and/or triethylammonium acetate.

125. The method of any one of claims 67-124, wherein the at least one mobile phase comprises 25 mM to 300 mM dibutylammonium acetate, optionally wherein the mobile phase comprises 50 mM dibutylammonium acetate.

126. The method of any one of claims 67-125, wherein the at least one mobile phase comprises 25 mM to 300 mM triethylammonium acetate, optionally wherein the mobile phase comprises 100 mM triethylammonium acetate.

127. The method of any one of claims 67-126, wherein the at least one mobile phase comprises 25% to 75% acetonitrile in water, optionally wherein the mobile phase comprises 50% acetonitrile in water.

128. The method of any one of claims 67-127, wherein the method comprises measuring absorbance of ion pair chromatography eluate at 260 nm.

129. The method of any one of claims 67-128, wherein the ion pair chromatography is carried out at a temperature of 20-80° C., optionally wherein the ion pair chromatography is carried out at a temperature of 65° C.

130. A kit for separating nucleic acid present in a lipid nucleic acid assembly composition, the kit comprising a ion pair chromatography substrate and at least one reagent for ethanol precipitation of the nucleic acid from the lipid nucleic acid assembly composition, optionally wherein the kit is for separating two or more nucleic acids, optionally wherein the two or more nucleic acid molecules are or include a gRNA and an mRNA, and/or for separating three or more nucleic acids, optionally wherein the three or more nucleic acid molecules are or include a gRNA, a first mRNA, and a second mRNA.

131. The kit of claim 130, wherein the ion pair chromatography substrate is, or is present in, an ion pair chromatography column.

132. The kit of claim 131, wherein the ion pair chromatography column has an internal diameter of 1 mm to 4 mm, optionally an internal diameter of about 1.5 mm to about 2.5 mm, optionally an internal diameter of about 2.1 mm internal diameter.

133. The kit of claim 131 or 132, wherein the ion pair chromatography column has a length of about 50 mm to about 400 mm, optionally a length of about 100 mm length.

134. The kit of any one of claims 130-133, wherein the ion pair chromatography substrate and/or ion pair chromatography column has a pore size of about 100 Å to about 3000 Å, optionally wherein the pore size is about 1000 Å to about 2000 Å.

135. The kit of any one of claims 130-134, wherein the ion pair chromatography substrate and/or ion pair chromatography column has a particle size of about 3 μm to about 5 μm, optionally a particle size of about 4 μm.

136. The method of any one of claims 130-135, wherein the ion pair chromatography substrate and/or ion pair chromatography column comprises silica, optionally wherein the ion pair chromatography substrate comprises silica particles.

137. The kit of any one of claims 130-136, wherein the ion pair chromatography substrate and/or ion pair chromatography column is compatible with a mobile phase comprising dibutylammonium acetate and/or triethylammonium acetate.

138. The kit of any one of claims 130-137, wherein the kit comprises a dilution series of one or more reference nucleic acids, optionally wherein the kit comprises a dilution series of a reference gRNA molecule, a first reference mRNA molecule, and/or a second reference mRNA molecule.

139. The kit of any one of claims 130-138, wherein the kit comprises instructions for preparation of a nucleic acid extract by a process comprising ethanol precipitation.

140. The kit of any one of claims 130-139, wherein the kit comprises instructions for separating the two or more nucleic acids. or three or more nucleic acids, by ion pair chromatography, optionally wherein the nucleic acids comprise or consist of RNA molecules, optionally wherein the RNA molecules comprise or consist of a gRNA molecule, a first mRNA molecule, and/or a second mRNA molecule.

141. The kit of any one of claims 130-140, wherein the kit comprises instructions for determining the concentration of the two more nucleic acids, three or more nucleic acids, and/or total nucleic acid concentration, optionally wherein the nucleic acids comprise or consist of RNA molecules, optionally wherein the RNA molecules comprise or consist of a gRNA molecule, a first mRNA molecule, and/or a second mRNA molecule.
